SEQUENCE LISTING (156 sequences)

SEQ ID NO: 1 - protein, 131 aa, synthetic construct
EVHLVESGGGLVQPGGSLRLSCAASGFTFSRYWMSWVRQAQGKGLEWVASIKQAGSEKTYVDSVKGRFTISRDNAKNSLSLQMNSLRAEDTAVYYCAREGAYYYDSASYYPYYYYYSMDVWGQGTTVTVSS

SEQ ID NO: 2 - protein, 108 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSQYTFGQGTKLEIK

SEQ ID NO: 3 - protein, 264 aa, synthetic construct
EVHLVESGGGLVQPGGSLRLSCAASGFTFSRYWMSWVRQAQGKGLEWVASIKQAGSEKTYVDSVKGRFTISRDNAKNSLSLQMNSLRAEDTAVYYCAREGAYYYDSASYYPYYYYYSMDVWGQGTTVTVSSGGGGSGGGGSGGGGSEIVLTQSPGTLSLSPGERATLSCRASQSVSSSYLAWYQQKPGQAPRLLIYGASSRATGIPDRFSGSGSGTDFTLTISRLEPEDFAVYYCQQYGSSQYTFGQGTKLEIKHHHHHHHHHH

SEQ ID NO: 4 - protein, 254 aa, synthetic construct
EVHLVESGGGLVQPGGSLRLSCAASGFTFSRYWMSWVRQAQGKGLEWVASIKQAGSEKTYVDSVKGRFTISRDNAKNSLSLQMNSLRAEDTAVYYCAREGAYYYDSASYYPYYYYYSMDVWGQGTTVTVSSGGGGSGGGGSGGGGSEIVLTQSPGTLSLSPGERATLSCRASQSVSSSYLAWYQQKPGQAPRLLIYGASSRATGIPDRFSGSGSGTDFTLTISRLEPEDFAVYYCQQYGSSQYTFGQGTKLEIK

SEQ ID NO: 5 - protein, 288 aa, synthetic construct
MGWSCIILFLVATATGVHSEVHLVESGGGLVQPGGSLRLSCAASGFTFSRYWMSWVRQAQGKGLEWVASIKQAGSEKTYVDSVKGRFTISRDNAKNSLSLQMNSLRAEDTAVYYCAREGAYYYDSASYYPYYYYYSMDVWGQGTTVTVSSGGGGSGGGGSGGGGSGGGGSEIVLTQSPGTLSLSPGERATLSCRASQSVSSSYLAWYQQKPGQAPRLLIYGASSRATGIPDRFSGSGSGTDFTLTISRLEPEDFAVYYCQQYGSSQYTFGQGTKLEIKHHHHHHHHHH

SEQ ID NO: 6 - protein, 332 aa, synthetic construct
EVHLVESGGGLVQPGGSLRLSCAASGFTFSRYWMSWVRQAQGKGLEWVASIKQAGSEKTYVDSVKGRFTISRDNAKNSLSLQMNSLRAEDTAVYYCAREGAYYYDSASYYPYYYYYSMDVWGQGTTVTVSSGGGGSGGGGSGGGGSEIVLTQSPGTLSLSPGERATLSCRASQSVSSSYLAWYQQKPGQAPRLLIYGASSRATGIPDRFSGSGSGTDFTLTISRLEPEDFAVYYCQQYGSSQYTFGQGTKLEIKGGGGSDYKDDDDKLSNSIMYFSHFVPVFLPAKPTTTPAPRPPTPAPTIASQPLSLRPEACRPAAGGAVHTRGLDFACD

SEQ ID NO: 7 - protein, 514 aa, synthetic construct
EVHLVESGGGLVQPGGSLRLSCAASGFTFSRYWMSWVRQAQGKGLEWVASIKQAGSEKTYVDSVKGRFTISRDNAKNSLSLQMNSLRAEDTAVYYCAREGAYYYDSASYYPYYYYYSMDVWGQGTTVTVSSGGGGSGGGGSGGGGSEIVLTQSPGTLSLSPGERATLSCRASQSVSSSYLAWYQQKPGQAPRLLIYGASSRATGIPDRFSGSGSGTDFTLTISRLEPEDFAVYYCQQYGSSQYTFGQGTKLEIKGGGGSDYKDDDDKLSNSIMYFSHFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 8 - protein, 514 aa, synthetic construct
EVHLVESGGGLVQPGGSLRLSCAASGFTFSRYWMSWVRQAQGKGLEWVASIKQAGSEKTYVDSVKGRFTISRDNAKNSLSLQMNSLRAEDTAVYYCAREGAYYYDSASYYPYYYYYSMDVWGQGTTVTVSSGGGGSGGGGSGGGGSEIVLTQSPGTLSLSPGERATLSCRASQSVSSSYLAWYQQKPGQAPRLLIYGASSRATGIPDRFSGSGSGTDFTLTISRLEPEDFAVYYCQQYGSSQYTFGQGTKLEIKGGGGSDYKDDDDKLSNSIMYFSHFVPVFLPAKPTTTPAPRPPTPAPTIASQPLSLRPEACRPAAGGAVHTRGLDFACDIYIWAPLAGTCGVLLLSLVITLYCNHRNKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 9 - protein, 15 aa, synthetic construct
GGGGSGGGGSGGGGS

SEQ ID NO: 10 - protein, 5 aa, synthetic construct
GGGGS

SEQ ID NO: 11 - protein, 8 aa, synthetic construct
DYKDDDDK

SEQ ID NO: 12 - protein, 93 aa, synthetic construct
LSNSIMYFSHFVPVFLPAKPTTTPAPRPPTPAPTIASQPLSLRPEACRPAAGGAVHTRGLDFACDIYIWAPLAGTCGVLLLSLVITLYCNHRN

SEQ ID NO: 13 - protein, 65 aa, synthetic construct
LSNSIMYFSHFVPVFLPAKPTTTPAPRPPTPAPTIASQPLSLRPEACRPAAGGAVHTRGLDFACD

SEQ ID NO: 14 - protein, 28 aa, synthetic construct
IYIWAPLAGTCGVLLLSLVITLYCNHRN

SEQ ID NO: 15 - protein, 41 aa, synthetic construct
RSKRSRLLHSDYMNMTPRRPGPTRKHYQPYAPPRDFAAYRS

SEQ ID NO: 16 - protein, 41 aa, synthetic construct
KRGRKKLLYIFKQPFMRPVQTTQEEDGCSCRFPEEEEGGCE

SEQ ID NO: 17 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 18 - protein, 73 aa, synthetic construct
TTTPAPRPPTPAPTIASQPLSLRPEACRPAAGGAVHTRGLDFACDIYIWAPLAGTCGVLLLSLVITLYCNHRN

SEQ ID NO: 19 - protein, 45 aa, synthetic construct
TTTPAPRPPTPAPTIASQPLSLRPEACRPAAGGAVHTRGLDFACD

SEQ ID NO: 20 - protein, 24 aa, synthetic construct
IYIWAPLAGTCGVLLLSLVITLYC

SEQ ID NO: 21 - protein, 113 aa, synthetic construct
LRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 22 - protein, 25 aa, synthetic construct
EVHLVESGGGLVQPGGSLRLSCAAS

SEQ ID NO: 23 - protein, 30 aa, synthetic construct
EVHLVESGGGLVQPGGSLRLSCAASGFTFS

SEQ ID NO: 24 - protein, 29 aa, synthetic construct
EVHLVESGGGLVQPGGSLRLSCAASGFTF

SEQ ID NO: 25 - protein, 7 aa, synthetic construct
GFTFSRY

SEQ ID NO: 26 - protein, 10 aa, synthetic construct
GFTFSRYWMS

SEQ ID NO: 27 - protein, 5 aa, synthetic construct
RYWMS

SEQ ID NO: 28 - protein, 6 aa, synthetic construct
SRYWMS

SEQ ID NO: 29 - protein, 8 aa, synthetic construct
GFTFSRYW

SEQ ID NO: 30 - protein, 19 aa, synthetic construct
WMSWVRQAQGKGLEWVASI

SEQ ID NO: 31 - protein, 14 aa, synthetic construct
WVRQAQGKGLEWVA

SEQ ID NO: 32 - protein, 11 aa, synthetic construct
WVRQAQGKGLE

SEQ ID NO: 33 - protein, 17 aa, synthetic construct
MSWVRQAQGKGLEWVAS

SEQ ID NO: 34 - protein, 6 aa, synthetic construct
KQAGSE

SEQ ID NO: 35 - protein, 10 aa, synthetic construct
SIKQAGSEKT

SEQ ID NO: 36 - protein, 17 aa, synthetic construct
SIKQAGSEKTYVDSVKG

SEQ ID NO: 37 - protein, 13 aa, synthetic construct
WVASIKQAGSEKT

SEQ ID NO: 38 - protein, 8 aa, synthetic construct
IKQAGSEK

SEQ ID NO: 39 - protein, 41 aa, synthetic construct
KTYVDSVKGRFTISRDNAKNSLSLQMNSLRAEDTAVYYCAR

SEQ ID NO: 40 - protein, 39 aa, synthetic construct
YVDSVKGRFTISRDNAKNSLSLQMNSLRAEDTAVYYCAR

SEQ ID NO: 41 - protein, 32 aa, synthetic construct
RFTISRDNAKNSLSLQMNSLRAEDTAVYYCAR

SEQ ID NO: 42 - protein, 37 aa, synthetic construct
YVDSVKGRFTISRDNAKNSLSLQMNSLRAEDTAVYYC

SEQ ID NO: 43 - protein, 38 aa, synthetic construct
TYVDSVKGRFTISRDNAKNSLSLQMNSLRAEDTAVYYC

SEQ ID NO: 44 - protein, 24 aa, synthetic construct
RFTISRDNAKNSLSLQMNSLRAED

SEQ ID NO: 45 - protein, 22 aa, synthetic construct
EGAYYYDSASYYPYYYYYSMDV

SEQ ID NO: 46 - protein, 23 aa, synthetic construct
AREGAYYYDSASYYPYYYYYSMD

SEQ ID NO: 47 - protein, 24 aa, synthetic construct
AREGAYYYDSASYYPYYYYYSMDV

SEQ ID NO: 48 - protein, 30 aa, synthetic construct
TAVYYCAREGAYYYDSASYYPYYYYYSMDV

SEQ ID NO: 49 - protein, 11 aa, synthetic construct
WGQGTTVTVSS

SEQ ID NO: 50 - protein, 12 aa, synthetic construct
VWGQGTTVTVSS

SEQ ID NO: 51 - protein, 23 aa, synthetic construct
EIVLTQSPGTLSLSPGERATLSC

SEQ ID NO: 52 - protein, 29 aa, synthetic construct
EIVLTQSPGTLSLSPGERATLSCRASQSV

SEQ ID NO: 53 - protein, 26 aa, synthetic construct [sequence omitted]

SEQ ID NO: 54 - protein, 20 aa, synthetic construct
EIVLTQSPGTLSLSPGERAT

SEQ ID NO: 55 - protein, 12 aa, synthetic construct
RASQSVSSSYLA

SEQ ID NO: 56 - protein, 8 aa, synthetic construct
SSSYLAWY

SEQ ID NO: 57 - protein, 7 aa, synthetic construct
QSVSSSY

SEQ ID NO: 58 - protein, 15 aa, synthetic construct
LSCRASQSVSSSYLA

SEQ ID NO: 59 - protein, 15 aa, synthetic construct
WYQQKPGQAPRLLIY

SEQ ID NO: 60 - protein, 9 aa, synthetic construct
QQKPGQAPR

SEQ ID NO: 61 - protein, 17 aa, synthetic construct
LAWYQQKPGQAPRLLIY

SEQ ID NO: 62 - protein, 7 aa, synthetic construct
GASSRAT

SEQ ID NO: 63 - protein, 10 aa, synthetic construct
LLIYGASSRA

SEQ ID NO: 64 - skipped

SEQ ID NO: 65 - protein, 32 aa, synthetic construct
GIPDRFSGSGSGTDFTLTISRLEPEDFAVYYC

SEQ ID NO: 66 - protein, 33 aa, synthetic construct
TGIPDRFSGSGSGTDFTLTISRLEPEDFAVYYC

SEQ ID NO: 67 - protein, 37 aa, synthetic construct
SSRATGIPDRFSGSGSGTDFTLTISRLEPEDFAVYYC

SEQ ID NO: 68 - protein, 9 aa, synthetic construct
QQYGSSQYT

SEQ ID NO: 69 - protein, 8 aa, synthetic construct
QQYGSSQY

SEQ ID NO: 70 - protein, 10 aa, synthetic construct
FGQGTKLEIK

SEQ ID NO: 71 - protein, 11 aa, synthetic construct
TFGQGTKLEIK

SEQ ID NO: 72 - DNA, 393 bp, synthetic construct
  source 1..393: mol_type = other DNA
gaggtgcacctggtggagagtggtggcggtctggtgcagccaggtggtagtctgaggctgagctgcgcagcaagcggcttcaccttcagcaggtactggatgagctgggtgaggcaggcccagggcaagggcctggagtgggtggccagcatcaagcaggccggcagcgagaagacctacgtggacagcgtgaagggcaggttcaccatcagcagggacaacgccaagaacagcctgagcctgcagatgaacagcctgagggccgaggacaccgccgtgtactactgcgccagggagggcgcctactactacgacagcgccagctactacccctactactactactacagcatggacgtgtggggccagggcaccaccgtgaccgtgagcagc

SEQ ID NO: 73 - DNA, 324 bp, synthetic construct
  source 1..324: mol_type = other DNA
gagatcgtgctgacccagagccccggcaccctgagcctgagccccggcgagagggccaccctgagctgcagggccagccagagcgtgagcagcagctacctggcctggtaccagcagaagcccggccaggcccccaggctgctgatctacggcgccagcagcagggccaccggcatccccgacaggttcagcggcagcggcagcggcaccgacttcaccctgaccatcagcaggctggagcccgaggacttcgccgtgtactactgccagcagtacggcagcagccagtacaccttcggccagggcaccaagctggagatcaag

SEQ ID NO: 74 - DNA, 792 bp, synthetic construct
  source 1..792: mol_type = other DNA
gaggtgcacctggtggagagtggtggcggtctggtgcagccaggtggtagtctgaggctgagctgcgcagcaagcggcttcaccttcagcaggtactggatgagctgggtgaggcaggcccagggcaagggcctggagtgggtggccagcatcaagcaggccggcagcgagaagacctacgtggacagcgtgaagggcaggttcaccatcagcagggacaacgccaagaacagcctgagcctgcagatgaacagcctgagggccgaggacaccgccgtgtactactgcgccagggagggcgcctactactacgacagcgccagctactacccctactactactactacagcatggacgtgtggggccagggcaccaccgtgaccgtgagcagcggcggtggcggaagcggtggtggcggtagcggaggtggcggcagcgagatcgtgctgacccagagccccggcaccctgagcctgagccccggcgagagggccaccctgagctgcagggccagccagagcgtgagcagcagctacctggcctggtaccagcagaagcccggccaggcccccaggctgctgatctacggcgccagcagcagggccaccggcatccccgacaggttcagcggcagcggcagcggcaccgacttcaccctgaccatcagcaggctggagcccgaggacttcgccgtgtactactgccagcagtacggcagcagccagtacaccttcggccagggcaccaagctggagatcaagcaccaccaccaccaccaccaccaccaccac

SEQ ID NO: 75 - DNA, 762 bp, synthetic construct
  source 1..762: mol_type = other DNA
gaggtgcacctggtggagagtggtggcggtctggtgcagccaggtggtagtctgaggctgagctgcgcagcaagcggcttcaccttcagcaggtactggatgagctgggtgaggcaggcccagggcaagggcctggagtgggtggccagcatcaagcaggccggcagcgagaagacctacgtggacagcgtgaagggcaggttcaccatcagcagggacaacgccaagaacagcctgagcctgcagatgaacagcctgagggccgaggacaccgccgtgtactactgcgccagggagggcgcctactactacgacagcgccagctactacccctactactactactacagcatggacgtgtggggccagggcaccaccgtgaccgtgagcagcggcggtggcggaagcggtggtggcggtagcggaggtggcggcagcgagatcgtgctgacccagagccccggcaccctgagcctgagccccggcgagagggccaccctgagctgcagggccagccagagcgtgagcagcagctacctggcctggtaccagcagaagcccggccaggcccccaggctgctgatctacggcgccagcagcagggccaccggcatccccgacaggttcagcggcagcggcagcggcaccgacttcaccctgaccatcagcaggctggagcccgaggacttcgccgtgtactactgccagcagtacggcagcagccagtacaccttcggccagggcaccaagctggagatcaag

SEQ ID NO: 76 - DNA, 849 bp, synthetic construct
  source 1..849: mol_type = other DNA
atgggctggagctgcatcatcctgttcctggtggccaccgccaccggcgtgcacagcgaggtgcacctggtggagagtggtggcggtctggtgcagccaggtggtagtctgaggctgagctgcgcagcaagcggcttcaccttcagcaggtactggatgagctgggtgaggcaggcccagggcaagggcctggagtgggtggccagcatcaagcaggccggcagcgagaagacctacgtggacagcgtgaagggcaggttcaccatcagcagggacaacgccaagaacagcctgagcctgcagatgaacagcctgagggccgaggacaccgccgtgtactactgcgccagggagggcgcctactactacgacagcgccagctactacccctactactactactacagcatggacgtgtggggccagggcaccaccgtgaccgtgagcagcggcggtggcggaagcggtggtggcggtagcggaggtggcggcagcgagatcgtgctgacccagagccccggcaccctgagcctgagccccggcgagagggccaccctgagctgcagggccagccagagcgtgagcagcagctacctggcctggtaccagcagaagcccggccaggcccccaggctgctgatctacggcgccagcagcagggccaccggcatccccgacaggttcagcggcagcggcagcggcaccgacttcaccctgaccatcagcaggctggagcccgaggacttcgccgtgtactactgccagcagtacggcagcagccagtacaccttcggccagggcaccaagctggagatcaagcaccaccaccaccaccaccaccaccaccac

SEQ ID NO: 77 - DNA, 996 bp, synthetic construct
  source 1..996: mol_type = other DNA
gaggtgcacctggtggagagtggtggcggtctggtgcagccaggtggtagtctgaggctgagctgcgcagcaagcggcttcaccttcagcaggtactggatgagctgggtgaggcaggcccagggcaagggcctggagtgggtggccagcatcaagcaggccggcagcgagaagacctacgtggacagcgtgaagggcaggttcaccatcagcagggacaacgccaagaacagcctgagcctgcagatgaacagcctgagggccgaggacaccgccgtgtactactgcgccagggagggcgcctactactacgacagcgccagctactacccctactactactactacagcatggacgtgtggggccagggcaccaccgtgaccgtgagcagcggcggtggcggaagcggtggtggcggtagcggaggtggcggcagcgagatcgtgctgacccagagccccggcaccctgagcctgagccccggcgagagggccaccctgagctgcagggccagccagagcgtgagcagcagctacctggcctggtaccagcagaagcccggccaggcccccaggctgctgatctacggcgccagcagcagggccaccggcatccccgacaggttcagcggcagcggcagcggcaccgacttcaccctgaccatcagcaggctggagcccgaggacttcgccgtgtactactgccagcagtacggcagcagccagtacaccttcggccagggcaccaagctggagatcaagggcggcggcggcagcgattacaaggacgacgatgacaagctgagcaactccatcatgtacttcagccacttcgtgcccgtgttcctgcctgccaagcctacaacaacccctgctcctagacctcctacaccagctcctacaatcgccagccagcctctgtctctgaggcctgaagcttgtagacctgctgctggcggagccgtgcataccagaggactggatttcgcctgcgac

SEQ ID NO: 78 - DNA, 1542 bp, synthetic construct
  source 1..1542: mol_type = other DNA
gaggtgcacctggtggagagtggtggcggtctggtgcagccaggtggtagtctgaggctgagctgcgcagcaagcggcttcaccttcagcaggtactggatgagctgggtgaggcaggcccagggcaagggcctggagtgggtggccagcatcaagcaggccggcagcgagaagacctacgtggacagcgtgaagggcaggttcaccatcagcagggacaacgccaagaacagcctgagcctgcagatgaacagcctgagggccgaggacaccgccgtgtactactgcgccagggagggcgcctactactacgacagcgccagctactacccctactactactactacagcatggacgtgtggggccagggcaccaccgtgaccgtgagcagcggcggtggcggaagcggtggtggcggtagcggaggtggcggcagcgagatcgtgctgacccagagccccggcaccctgagcctgagccccggcgagagggccaccctgagctgcagggccagccagagcgtgagcagcagctacctggcctggtaccagcagaagcccggccaggcccccaggctgctgatctacggcgccagcagcagggccaccggcatccccgacaggttcagcggcagcggcagcggcaccgacttcaccctgaccatcagcaggctggagcccgaggacttcgccgtgtactactgccagcagtacggcagcagccagtacaccttcggccagggcaccaagctggagatcaagggcggcggcggcagcgattacaaggacgacgatgacaagctgagcaactccatcatgtacttcagccacttcgtgcccgtgttcctgcctgccaagcctacaacaacccctgctcctagacctcctacaccagctcctacaatcgccagccagcctctgtctctgaggcctgaagcttgtagacctgctgctggcggagccgtgcataccagaggactggatttcgcctgcgacatctacatctgggcccctctggctggaacatgtggcgttttgctgctgagcctcgttatcaccctgtactgcaaccaccggaacaggagtaagaggagcaggctcctgcacagtgactacatgaacatgactccccgccgccccgggcccacccgcaagcattaccagccctatgccccaccacgcgacttcgcagcctatcgctccctgagagtgaagttcagcagaagcgccgacgcccctgcctatcagcagggccagaaccagctgtataacgagctgaacctgggcagacgggaagagtacgacgtgctggacaagcggagaggcagggaccctgagatgggcggcaagcccagaagaaagaacccccaggaaggcctgtataacgaactgcagaaagacaagatggccgaggcctacagcgagatcggaatgaagggcgagcggagaagaggcaagggccacgatggcctgtaccagggcctgagcaccgccaccaaggacacctatgacgccctgcacatgcaggccctgccccccaga

SEQ ID NO: 79 - DNA, 1542 bp, synthetic construct
  source 1..1542: mol_type = other DNA
gaggtgcacctggtggagagtggtggcggtctggtgcagccaggtggtagtctgaggctgagctgcgcagcaagcggcttcaccttcagcaggtactggatgagctgggtgaggcaggcccagggcaagggcctggagtgggtggccagcatcaagcaggccggcagcgagaagacctacgtggacagcgtgaagggcaggttcaccatcagcagggacaacgccaagaacagcctgagcctgcagatgaacagcctgagggccgaggacaccgccgtgtactactgcgccagggagggcgcctactactacgacagcgccagctactacccctactactactactacagcatggacgtgtggggccagggcaccaccgtgaccgtgagcagcggcggtggcggaagcggtggtggcggtagcggaggtggcggcagcgagatcgtgctgacccagagccccggcaccctgagcctgagccccggcgagagggccaccctgagctgcagggccagccagagcgtgagcagcagctacctggcctggtaccagcagaagcccggccaggcccccaggctgctgatctacggcgccagcagcagggccaccggcatccccgacaggttcagcggcagcggcagcggcaccgacttcaccctgaccatcagcaggctggagcccgaggacttcgccgtgtactactgccagcagtacggcagcagccagtacaccttcggccagggcaccaagctggagatcaagggcggcggcggcagcgattacaaggacgacgatgacaagctgagcaactccatcatgtacttcagccacttcgtgcccgtgttcctgcctgccaagcctacaacaacccctgctcctagacctcctacaccagctcctacaatcgccagccagcctctgtctctgaggcctgaagcttgtagacctgctgctggcggagccgtgcataccagaggactggatttcgcctgcgacatctacatctgggcccctctggctggaacatgtggcgttttgctgctgagcctcgttatcaccctgtactgcaaccaccggaacaagcggggcagaaagaagctgctgtatatcttcaagcagcccttcatgcggcccgtgcagaccacccaggaagaggacggctgctcctgcagattccccgaggaagaagaaggcggctgcgagctgagagtgaagttcagcagaagcgccgacgcccctgcctatcagcagggccagaaccagctgtataacgagctgaacctgggcagacgggaagagtacgacgtgctggacaagcggagaggcagggaccctgagatgggcggcaagcccagaagaaagaacccccaggaaggcctgtataacgaactgcagaaagacaagatggccgaggcctacagcgagatcggaatgaagggcgagcggagaagaggcaagggccacgatggcctgtaccagggcctgagcaccgccaccaaggacacctatgacgccctgcacatgcaggccctgccccccaga

SEQ ID NO: 80 - DNA, 45 bp, synthetic construct
  source 1..45: mol_type = other DNA
ggcggtggcggaagcggtggtggcggtagcggaggtggcggcagc

SEQ ID NO: 81 - DNA, 15 bp, synthetic construct
  source 1..15: mol_type = other DNA
ggcggcggcggcagc

SEQ ID NO: 82 - DNA, 24 bp, synthetic construct
  source 1..24: mol_type = other DNA
gattacaaggacgacgatgacaag

SEQ ID NO: 83 - DNA, 279 bp, synthetic construct
  source 1..279: mol_type = other DNA
ctgagcaactccatcatgtacttcagccacttcgtgcccgtgttcctgcctgccaagcctacaacaacccctgctcctagacctcctacaccagctcctacaatcgccagccagcctctgtctctgaggcctgaagcttgtagacctgctgctggcggagccgtgcataccagaggactggatttcgcctgcgacatctacatctgggcccctctggctggaacatgtggcgttttgctgctgagcctcgttatcaccctgtactgcaaccaccggaac

SEQ ID NO: 84 - DNA, 195 bp, synthetic construct
  source 1..195: mol_type = other DNA
ctgagcaactccatcatgtacttcagccacttcgtgcccgtgttcctgcctgccaagcctacaacaacccctgctcctagacctcctacaccagctcctacaatcgccagccagcctctgtctctgaggcctgaagcttgtagacctgctgctggcggagccgtgcataccagaggactggatttcgcctgcgac

SEQ ID NO: 85 - DNA, 84 bp, synthetic construct
  source 1..84: mol_type = other DNA
atctacatctgggcccctctggctggaacatgtggcgttttgctgctgagcctcgttatcaccctgtactgcaaccaccggaac

SEQ ID NO: 86 - DNA, 123 bp, synthetic construct
  source 1..123: mol_type = other DNA
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 87 - DNA, 126 bp, synthetic construct
  source 1..126: mol_type = other DNA
aagcggggcagaaagaagctgctgtatatcttcaagcagcccttcatgcggcccgtgcagaccacccaggaagaggacggctgctcctgcagattccccgaggaagaagaaggcggctgcgagctg

SEQ ID NO: 88 - DNA, 339 bp, synthetic construct
  source 1..339: mol_type = other DNA
ctgagagtgaagttcagcagaagcgccgacgcccctgcctatcagcagggccagaaccagctgtataacgagctgaacctgggcagacgggaagagtacgacgtgctggacaagcggagaggcagggaccctgagatgggcggcaagcccagaagaaagaacccccaggaaggcctgtataacgaactgcagaaagacaagatggccgaggcctacagcgagatcggaatgaagggcgagcggagaagaggcaagggccacgatggcctgtaccagggcctgagcaccgccaccaaggacacctatgacgccctgcacatgcaggccctgccccccaga

SEQ ID NO: 89 - DNA, 291 bp, synthetic construct
  source 1..291: mol_type = other DNA
acaacaacccctgctcctagacctcctacaccagctcctacaatcgccagccagcctctgtctctgaggcctgaagcttgtagacctgctgctggcggagccgtgcataccagaggactggatttcgcctgcgacatctacatctgggcccctctggctggaacatgtggcgttttgctgctgagcctcgttatcaccctgtactgcaaccaccggaacatctacatctgggcccctctggctggaacatgtggcgttttgctgctgagcctcgttatcaccctgtactgc

SEQ ID NO: 90 - DNA, 219 bp, synthetic construct
  source 1..219: mol_type = other DNA
acaacaacccctgctcctagacctcctacaccagctcctacaatcgccagccagcctctgtctctgaggcctgaagcttgtagacctgctgctggcggagccgtgcataccagaggactggatttcgcctgcgacatctacatctgggcccctctggctggaacatgtggcgttttgctgctgagcctcgttatcaccctgtactgcaaccaccggaac

SEQ ID NO: 91 - DNA, 72 bp, synthetic construct
  source 1..72: mol_type = other DNA
atctacatctgggcccctctggctggaacatgtggcgttttgctgctgagcctcgttatcaccctgtactgc

SEQ ID NO: 92 - DNA, 336 bp, synthetic construct
  source 1..336: mol_type = other DNA
agagtgaagttcagcagaagcgccgacgcccctgcctatcagcagggccagaaccagctgtataacgagctgaacctgggcagacgggaagagtacgacgtgctggacaagcggagaggcagggaccctgagatgggcggcaagcccagaagaaagaacccccaggaaggcctgtataacgaactgcagaaagacaagatggccgaggcctacagcgagatcggaatgaagggcgagcggagaagaggcaagggccacgatggcctgtaccagggcctgagcaccgccaccaaggacacctatgacgccctgcacatgcaggccctgccccccaga

SEQ ID NO: 93 - DNA, 75 bp, synthetic construct
  source 1..75: mol_type = other DNA
gaggtgcacctggtggagagtggtggcggtctggtgcagccaggtggtagtctgaggctgagctgcgcagcaagc

SEQ ID NO: 94 - DNA, 90 bp, synthetic construct
  source 1..90: mol_type = other DNA
gaggtgcacctggtggagagtggtggcggtctggtgcagccaggtggtagtctgaggctgagctgcgcagcaagcggcttcaccttcagc

SEQ ID NO: 95 - DNA, 87 bp, synthetic construct
  source 1..87: mol_type = other DNA
gaggtgcacctggtggagagtggtggcggtctggtgcagccaggtggtagtctgaggctgagctgcgcagcaagcggcttcaccttc

SEQ ID NO: 96 - DNA, 21 bp, synthetic construct
  source 1..21: mol_type = other DNA
ggcttcaccttcagcaggtac

SEQ ID NO: 97 - DNA, 30 bp, synthetic construct
  source 1..30: mol_type = other DNA
ggcttcaccttcagcaggtactggatgagc

SEQ ID NO: 98 - DNA, 15 bp, synthetic construct
  source 1..15: mol_type = other DNA
aggtactggatgagc

SEQ ID NO: 99 - DNA, 18 bp, synthetic construct
  source 1..18: mol_type = other DNA
agcaggtactggatgagc

SEQ ID NO: 100 - DNA, 24 bp, synthetic construct
  source 1..24: mol_type = other DNA
ggcttcaccttcagcaggtactgg

SEQ ID NO: 101 - DNA, 57 bp, synthetic construct
  source 1..57: mol_type = other DNA
tggatgagctgggtgaggcaggcccagggcaagggcctggagtgggtggccagcatc

SEQ ID NO: 102 - DNA, 42 bp, synthetic construct
  source 1..42: mol_type = other DNA
tgggtgaggcaggcccagggcaagggcctggagtgggtggcc

SEQ ID NO: 103 - DNA, 33 bp, synthetic construct
  source 1..33: mol_type = other DNA
tgggtgaggcaggcccagggcaagggcctggag

SEQ ID NO: 104 - DNA, 51 bp, synthetic construct
  source 1..51: mol_type = other DNA
atgagctgggtgaggcaggcccagggcaagggcctggagtgggtggccagc

SEQ ID NO: 105 - DNA, 18 bp, synthetic construct
  source 1..18: mol_type = other DNA
aagcaggccggcagcgag

SEQ ID NO: 106 - DNA, 30 bp, synthetic construct
  source 1..30: mol_type = other DNA
agcatcaagcaggccggcagcgagaagacc

SEQ ID NO: 107 - DNA, 51 bp, synthetic construct
  source 1..51: mol_type = other DNA
agcatcaagcaggccggcagcgagaagacctacgtggacagcgtgaagggc

SEQ ID NO: 108 - DNA, 39 bp, synthetic construct
  source 1..39: mol_type = other DNA
tgggtggccagcatcaagcaggccggcagcgagaagacc

SEQ ID NO: 109 - DNA, 24 bp, synthetic construct
  source 1..24: mol_type = other DNA
atcaagcaggccggcagcgagaag

SEQ ID NO: 110 - DNA, 123 bp, synthetic construct
  source 1..123: mol_type = other DNA
aagacctacgtggacagcgtgaagggcaggttcaccatcagcagggacaacgccaagaacagcctgagcctgcagatgaacagcctgagggccgaggacaccgccgtgtactactgcgccagg

SEQ ID NO: 111 - DNA, 117 bp, synthetic construct
  source 1..117: mol_type = other DNA
tacgtggacagcgtgaagggcaggttcaccatcagcagggacaacgccaagaacagcctgagcctgcagatgaacagcctgagggccgaggacaccgccgtgtactactgcgccagg

SEQ ID NO: 112 - DNA, 96 bp, synthetic construct
  source 1..96: mol_type = other DNA
aggttcaccatcagcagggacaacgccaagaacagcctgagcctgcagatgaacagcctgagggccgaggacaccgccgtgtactactgcgccagg

SEQ ID NO: 113 - DNA, 111 bp, synthetic construct
  source 1..111: mol_type = other DNA
tacgtggacagcgtgaagggcaggttcaccatcagcagggacaacgccaagaacagcctgagcctgcagatgaacagcctgagggccgaggacaccgccgtgtactactgc

SEQ ID NO: 114 - DNA, 114 bp, synthetic construct
  source 1..114: mol_type = other DNA
acctacgtggacagcgtgaagggcaggttcaccatcagcagggacaacgccaagaacagcctgagcctgcagatgaacagcctgagggccgaggacaccgccgtgtactactgc

SEQ ID NO: 115 - DNA, 72 bp, synthetic construct
  source 1..72: mol_type = other DNA
aggttcaccatcagcagggacaacgccaagaacagcctgagcctgcagatgaacagcctgagggccgaggac

SEQ ID NO: 116 - DNA, 66 bp, synthetic construct
  source 1..66: mol_type = other DNA
gagggcgcctactactacgacagcgccagctactacccctactactactactacagcatggacgtg

SEQ ID NO: 117 - DNA, 69 bp, synthetic construct
  source 1..69: mol_type = other DNA
gccagggagggcgcctactactacgacagcgccagctactacccctactactactactacagcatggac

SEQ ID NO: 118 - DNA, 72 bp, synthetic construct
  source 1..72: mol_type = other DNA
gccagggagggcgcctactactacgacagcgccagctactacccctactactactactacagcatggacgtg

SEQ ID NO: 119 - DNA, 90 bp, synthetic construct
  source 1..90: mol_type = other DNA
accgccgtgtactactgcgccagggagggcgcctactactacgacagcgccagctactacccctactactactactacagcatggacgtg

SEQ ID NO: 120 - DNA, 33 bp, synthetic construct
  source 1..33: mol_type = other DNA
tggggccagggcaccaccgtgaccgtgagcagc

SEQ ID NO: 121 - DNA, 36 bp, synthetic construct
  source 1..36: mol_type = other DNA
gtgtggggccagggcaccaccgtgaccgtgagcagc

SEQ ID NO: 122 - DNA, 69 bp, synthetic construct
  source 1..69: mol_type = other DNA
gagatcgtgctgacccagagccccggcaccctgagcctgagccccggcgagagggccaccctgagctgc

SEQ ID NO: 123 - DNA, 87 bp, synthetic construct
  source 1..87: mol_type = other DNA
gagatcgtgctgacccagagccccggcaccctgagcctgagccccggcgagagggccaccctgagctgcagggccagccagagcgtg

SEQ ID NO: 124 - DNA, 78 bp, synthetic construct
  source 1..78: mol_type = other DNA
gagatcgtgctgacccagagccccggcaccctgagcctgagccccggcgagagggccaccctgagctgcagggccagc

SEQ ID NO: 125 - DNA, 60 bp, synthetic construct
  source 1..60: mol_type = other DNA
gagatcgtgctgacccagagccccggcaccctgagcctgagccccggcgagagggccacc

SEQ ID NO: 126 - DNA, 36 bp, synthetic construct
  source 1..36: mol_type = other DNA
agggccagccagagcgtgagcagcagctacctggcc

SEQ ID NO: 127 - DNA, 24 bp, synthetic construct
  source 1..24: mol_type = other DNA
agcagcagctacctggcctggtac

SEQ ID NO: 128 - DNA, 21 bp, synthetic construct
  source 1..21: mol_type = other DNA
cagagcgtgagcagcagctac

SEQ ID NO: 129 - DNA, 45 bp, synthetic construct
  source 1..45: mol_type = other DNA
ctgagctgcagggccagccagagcgtgagcagcagctacctggcc

SEQ ID NO: 130 - DNA, 45 bp, synthetic construct
  source 1..45: mol_type = other DNA
tggtaccagcagaagcccggccaggcccccaggctgctgatctac

SEQ ID NO: 131 - DNA, 27 bp, synthetic construct
  source 1..27: mol_type = other DNA
cagcagaagcccggccaggcccccagg

SEQ ID NO: 132 - DNA, 51 bp, synthetic construct
  source 1..51: mol_type = other DNA
ctggcctggtaccagcagaagcccggccaggcccccaggctgctgatctac

SEQ ID NO: 133 - DNA, 21 bp, synthetic construct
  source 1..21: mol_type = other DNA
ggcgccagcagcagggccacc

SEQ ID NO: 134 - DNA, 33 bp, synthetic construct
  source 1..33: mol_type = other DNA
ctgctgatctacggcgccagcagcagggccacc

SEQ ID NO: 135 - skipped

SEQ ID NO: 136 - DNA, 96 bp, synthetic construct
  source 1..96: mol_type = other DNA
ggcatccccgacaggttcagcggcagcggcagcggcaccgacttcaccctgaccatcagcaggctggagcccgaggacttcgccgtgtactactgc

SEQ ID NO: 137 - DNA, 99 bp, synthetic construct
  source 1..99: mol_type = other DNA
accggcatccccgacaggttcagcggcagcggcagcggcaccgacttcaccctgaccatcagcaggctggagcccgaggacttcgccgtgtactactgc

SEQ ID NO: 138 - DNA, 111 bp, synthetic construct
  source 1..111: mol_type = other DNA
agcagcagggccaccggcatccccgacaggttcagcggcagcggcagcggcaccgacttcaccctgaccatcagcaggctggagcccgaggacttcgccgtgtactactgc

SEQ ID NO: 139 - DNA, 27 bp, synthetic construct
  source 1..27: mol_type = other DNA
cagcagtacggcagcagccagtacacc

SEQ ID NO: 140 - DNA, 24 bp, synthetic construct
  source 1..24: mol_type = other DNA
cagcagtacggcagcagccagtac

SEQ ID NO: 141 - DNA, 30 bp, synthetic construct
  source 1..30: mol_type = other DNA
ttcggccagggcaccaagctggagatcaag

SEQ ID NO: 142 - DNA, 33 bp, synthetic construct
  source 1..33: mol_type = other DNA
accttcggccagggcaccaagctggagatcaag

SEQ ID NO: 143 - protein, 10 aa, synthetic construct
GDYKDDDDKG

SEQ ID NO: 144 - protein, 9 aa, synthetic construct
MDYKDDDDK

SEQ ID NO: 145 - protein, 7 aa, synthetic construct
DFKDDDK

SEQ ID NO: 146 - protein, 8 aa, synthetic construct
DYKAFDNL

SEQ ID NO: 147 - protein, 7 aa, synthetic construct
DYKDHDG

SEQ ID NO: 148 - protein, 9 aa, synthetic construct
MDFKDDDDK

SEQ ID NO: 149 - protein, 9 aa, synthetic construct
MDYKAFDNL

SEQ ID NO: 150 - protein, 7 aa, synthetic construct
DYKDHDI

SEQ ID NO: 151 - protein, 5 aa, synthetic construct
DYKDH

SEQ ID NO: 152 - protein, 5 aa, synthetic construct
DYKDD

SEQ ID NO: 153 - protein, 6 aa, synthetic construct
DYKDHD

SEQ ID NO: 154 - protein, 6 aa, synthetic construct
DYKDDD

SEQ ID NO: 155 - DNA, 8047 bp, synthetic construct
  source 1..8047: mol_type = other DNA
aacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcgatactagtggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctactctagagctagcgaattctccaccatggattttcaggtgcagattttcagcttcctgctaatcagtgcctcagtcataatgtctagagaggtgcacctggtggagagtggtggcggtctggtgcagccaggtggtagtctgaggctgagctgcgcagcaagcggcttcaccttcagcaggtactggatgagctgggtgaggcaggcccagggcaagggcctggagtgggtggccagcatcaagcaggccggcagcgagaagacctacgtggacagcgtgaagggcaggttcaccatcagcagggacaacgccaagaacagcctgagcctgcagatgaacagcctgagggccgaggacaccgccgtgtactactgcgccagggagggcgcctactactacgacagcgccagctactacccctactactactactacagcatggacgtgtggggccagggcaccaccgtgaccgtgagcagcggcggtggcggaagcggtggtggcggtagcggaggtggcggcagcgagatcgtgctgacccagagccccggcaccctgagcctgagccccggcgagagggccaccctgagctgcagggccagccagagcgtgagcagcagctacctggcctggtaccagcagaagcccggccaggcccccaggctgctgatctacggcgccagcagcagggccaccggcatccccgacaggttcagcggcagcggcagcggcaccgacttcaccctgaccatcagcaggctggagcccgaggacttcgccgtgtactactgccagcagtacggcagcagccagtacaccttcggccagggcaccaagctggagatcaagggcggcggcggcagcgattacaaggacgacgatgacaagctgagcaactccatcatgtacttcagccacttcgtgcccgtgttcctgcctgccaagcctacaacaacccctgctcctagacctcctacaccagctcctacaatcgccagccagcctctgtctctgaggcctgaagcttgtagacctgctgctggcggagccgtgcataccagaggactggatttcgcctgcgacatctacatctgggcccctctggctggaacatgtggcgttttgctgctgagcctcgtgatcaccctgtactgcaaccaccggaacaggagtaagaggagcaggctcctgcacagtgactacatgaacatgactccccgccgccccgggcccacccgcaagcattaccagccctatgccccaccacgcgacttcgcagcctatcgctccctgagagtgaagttcagcagaagcgccgacgcccctgcctatcagcagggccagaaccagctgtacaacgagctgaacctgggcagacgggaagagtacgacgtgctggacaagcggagaggcagggaccctgagatgggcggcaagcccagaagaaagaacccccaggaaggcctgtataacgaactgcagaaagacaagatggccgaggcctacagcgagatcggaatgaagggcgagcggagaagaggcaagggccacgatggcctgtaccagggcctgagcaccgccaccaaggacacctatgacgccctgcacatgcaggccctgccccccagaggatccgcggcctcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggtacctttaagaccaatgacttacaaggcagctgtagatcttagccactttttaaaagaaaaggggggactggaagggctaattcactcccaacgaagataagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagacttttgcagagaccaaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctgacgcgtgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccactgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtg

SEQ ID NO: 156 - DNA, 1605 bp, synthetic construct
  source 1..1605: mol_type = other DNA
atggattttcaagtccaaatatttagttttctcttgattagcgcaagcgtgataatgagccgcgaggtccatcttgtcgagtccggaggtggtttggtgcaacctggcggctccttgcggctctcctgtgctgcgtctggcttcacgttttctaggtactggatgagctgggtacgccaggcccaaggaaaggggcttgagtgggtagctagtataaagcaggcgggatccgagaaaacttacgtcgactctgtcaaaggacgctttaccatatcccgcgataacgcaaaaaattcactgagtctgcagatgaactctctgagagcagaagatacagctgtgtattattgcgcgcgagaaggagcatattattatgattccgcatcatactatccatactactattactactcaatggatgtctggggccagggcacaaccgttacggttagcagcggtgggggtggctccgggggcgggggctcaggaggcggtggcagcgaaattgtattgacgcagagtccaggcactttgtctctgtctccaggggaaagggctacgttgtcatgccgagcttctcagtcagtaagtagttcctacctcgcctggtatcagcagaaaccgggccaagcaccaagacttctgatttatggggcgtcttccagggccaccggtatccctgacagattttccgggtccggaagtggaactgattttacccttaccattagtcgcctcgagccagaagactttgccgtgtattattgccagcagtacggtagttcccaatacacatttggccaaggcacgaaactggaaataaaaggcggtggcggatcagattataaggatgatgacgataaattgtcaaactctataatgtacttctcccactttgtacccgtatttctgcctgcaaaacccaccactaccccggcgcctcgacctcccacgccagctcctactatagcttcacagcctctctcactccgaccagaagcttgtagaccggccgcaggcggagctgtccacactagggggctcgactttgcttgtgatatctacatttgggctccacttgccggcacttgtggggtcctcctcttgagtctcgtaatcacactttattgcaatcatcggaatagatccaaacgatccagactgctgcactccgattatatgaacatgacgccacggagaccaggaccaacaaggaaacattaccagccatacgccccaccccgagactttgcagcctacaggtccctccgggttaaattcagcaggtctgccgatgctccagcataccagcaagggcagaatcaactttacaacgagcttaatcttggccggagggaagaatatgatgtgctggataagcggaggggccgagaccctgaaatgggagggaaaccgcgacgcaaaaaccctcaagaggggctttataacgagctgcaaaaggacaaaatggctgaggcttacagtgagataggcatgaaaggagagagaaggcgggggaaggggcacgacggcctgtatcaggggcttagcacggcaactaaggacacctacgacgctttgcatatgcaggcactcccgcctaga

(19) United States
(12) Patent Application Publication
(10) Pub. No.: US 2024/0101638 A1
(43) Pub. Date: Mar. 28, 2024

(54) NOVEL CAR

(71) Applicant: Currus Biologics Pty Ltd, Melbourne (AU)

(72) Inventors: Samantha Cobb, Melbourne (AU); Michael Kershaw, Melbourne (AU); Clare Slaney, Melbourne (AU)

(73) Assignee: Currus Biologics Pty Ltd, Melbourne (AU)

(21) Appl. No.: 17/933,574

(22) Filed: Sep. 20, 2022

(51) Int. Cl. C07K 14/725

(52) U.S. Cl. CPC C07K 14/7051; C07K 2317/53; C07K 2317/565; C07K 2319/02; C07K 2319/03

(57) ABSTRACT

The invention relates to a chimeric antigen receptor (CAR) that binds to mesothelin and cells that express the CAR, as well as uses thereof for the treatment of cancer.

### REFERENCE TO AN ELECTRONIC SEQUENCE LISTING

The contents of the electronic sequence listing (C1695700000US00-SEQ-KZM.xml; Size: 159,150 bytes; and Date of Creation: Sep. 20, 2022) is herein incorporated by reference in its entirety.

### FIELD OF THE INVENTION

The invention relates to a chimeric antigen receptor (CAR) that binds to mesothelin and cells that express the CAR, as well as uses thereof for the treatment of cancer.

### BACKGROUND OF THE INVENTION

Mesothelin was originally identified as a tumor associated antigen due to its limited expression by normal tissues and overexpression on tumors.

The mesothelin gene encodes a precursor 71-kDa protein that is processed to yield the 40-kDa protein, mesothelin, which is anchored at the cell membrane by a glycosylphosphatidyl inositol (GPI) linkage and an amino-terminal 31-kDa shed fragment, called megkaryocyte potentiating factor (MPF). Both fragments contain N-glycosylation sites. A soluble splice variant of the 40-kDa carboxyl-terminal fragment called “soluble mesothelin/MPF-related” has been found in the sera of patients with pancreatic ductal adenocarcinoma (PDA).

Mesothelin is a differentiation antigen that is also present on a restricted set of normal tissues. Using the mouse anti-human mesothelin antibody K1 that was developed by the Pastan group, strong K1 reactivity has been demonstrated within mesothelial cells that line the peritoneal, pleural, and pericardial cavities, although at lower levels than usually seen for malignant tissues. Weak K1 reactivity has been detected within the Fallopian tube epithelium, tracheal basal epithelium and tonsils epithelium. Mesothelin has also been found on all layers of the cornea. However, K1 reactivity has not been detected in the majority of normal tissues including the liver, kidneys, spleen, bone marrow, lymph nodes, thymus, cardiac muscle, tongue, skeletal muscle, skin, cerebral cortex, cerebellum, spinal cord, peripheral nerve, pituitary, adrenal, salivary gland, mammary gland, thyroid, parathyroid, testis, prostate, epididymis, cervical epithelium, lung parenchyma, esophagus, small-bowel epithelium, colon epithelium, bladder epithelium, gall-bladder epithelium.

There is a need for new and/or improved approaches for targeting mesothelin expressing cancers.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

### SUMMARY OF THE INVENTION

In one aspect, the present invention provides a chimeric antigen receptor (CAR) comprising:
(i) an antigen recognition domain that recognises mesothelin;
(ii) a transmembrane domain; and
(iii) an intracellular signalling domain,
wherein the antigen recognition domain comprises:
(a) a light chain variable domain (VL) comprising three complementarity determining regions (CDRs) of the amino acid sequence shown in SEQ ID NO: 2; and
(b) a heavy chain variable domain (VH) comprising three complementarity determining regions (CDRs) of the amino acid sequence shown in SEQ ID NO: 1.

In one aspect, the present invention provides a chimeric antigen receptor (CAR) comprising:
(i) an antigen recognition domain that recognises mesothelin;
(ii) a transmembrane domain; and
(iii) an intracellular signalling domain,
   wherein the antigen recognition domain comprises:
(a) a light chain variable domain (VL) comprising a complementarity determining region (CDR) 1 comprising or consisting of an amino acid sequence of SEQ ID NOs: 55, 56, 57 or 58; a CDR2 comprising or consisting of an amino acid sequence of SEQ ID NO: 62, 63 or 64; a CDR3 comprising or consisting of an amino acid sequence of SEQ ID NO: 68 or 69; and
(b) a heavy chain variable domain (VH) comprising a complementarity determining region (CDR) 1 comprising or consisting of an amino acid sequence of SEQ ID NO: 25, 26, 27, 28 or 29; a CDR2 comprising or consisting of an amino acid sequence of SEQ ID NO: 34, 35, 36, 37 or 38; a CDR3 comprising or consisting of an amino acid sequence of SEQ ID NO: 45, 46, 47 or 48.

In one embodiment, the antigen recognition domain comprises:
(a) a light chain variable domain (VL) comprising a complementarity determining region (CDR) 1 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NOs: 55; a CDR2 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 62; a CDR3 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of
(b) a heavy chain variable domain (VH) comprising a complementarity determining region (CDR) 1 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 27; a CDR2 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 36; a CDR3 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 45,
or
(c) a light chain variable domain (VL) comprising a complementarity determining region (CDR) 1 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NOs: 55; a CDR2 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 62; a CDR3 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of
(d) a heavy chain variable domain (VH) comprising a complementarity determining region (CDR) 1 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 25; a CDR2 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 34; a CDR3 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 45,
or
(e) a light chain variable domain (VL) comprising a complementarity determining region (CDR) 1 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NOs: 55; a CDR2 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 62; a CDR3 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of
(f) a heavy chain variable domain (VH) comprising a complementarity determining region (CDR) 1 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 26; a CDR2 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 35; a CDR3 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 45,
or
(g) a light chain variable domain (VL) comprising a complementarity determining region (CDR) 1 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NOs: 56; a CDR2 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 63; a CDR3 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 69; and
(h) a heavy chain variable domain (VH) comprising a complementarity determining region (CDR) 1 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 28; a CDR2 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 37; a CDR3 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 46,
or
(i) a light chain variable domain (VL) comprising a complementarity determining region (CDR) 1 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NOs: 57; a CDR2 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 64; a CDR3 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of
(j) a heavy chain variable domain (VH) comprising a complementarity determining region (CDR) 1 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 29; a CDR2 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 38; a CDR3 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 47,
or
(k) a light chain variable domain (VL) comprising a complementarity determining region (CDR) 1 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NOs: 58; a CDR2 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 62; a CDR3 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of
(l) a heavy chain variable domain (VH) comprising a complementarity determining region (CDR) 1 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 27; a CDR2 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 36; a CDR3 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 48.

In one embodiment, the antigen recognition domain comprises:
(a) a light chain variable domain (VL) comprising a complementarity determining region (CDR) 1 comprising or consisting of an amino acid sequence of SEQ ID NOs: 55; a CDR2 comprising or consisting of an amino acid sequence of SEQ ID NO: 62; a CDR3 comprising or consisting of an amino acid sequence of SEQ ID NO: 68; and
(b) a heavy chain variable domain (VH) comprising a complementarity determining region (CDR) 1 comprising or consisting of an amino acid sequence of SEQ ID NO: 27; a CDR2 comprising or consisting of an amino acid sequence of SEQ ID NO: 36; a CDR3 comprising or consisting of an amino acid sequence of SEQ ID NO: 45,
or
(c) a light chain variable domain (VL) comprising a complementarity determining region (CDR) 1 comprising or consisting of an amino acid sequence of SEQ ID NOs: 55; a CDR2 comprising or consisting of an amino acid sequence of SEQ ID NO: 62; a CDR3 comprising or consisting of an amino acid sequence of SEQ ID NO: 68; and
(d) a heavy chain variable domain (VH) comprising a complementarity determining region (CDR) 1 comprising or consisting of an amino acid sequence of SEQ ID NO: 25; a CDR2 comprising or consisting of an amino acid sequence of SEQ ID NO: 34; a CDR3 comprising or consisting of an amino acid sequence of SEQ ID NO: 45,
or
(e) a light chain variable domain (VL) comprising a complementarity determining region (CDR) 1 comprising or consisting of an amino acid sequence of SEQ ID NOs: 55; a CDR2 comprising or consisting of an amino acid sequence of SEQ ID NO: 62; a CDR3 comprising or consisting of an amino acid sequence of SEQ ID NO: 68; and
(f) a heavy chain variable domain (VH) comprising a complementarity determining region (CDR) 1 comprising or consisting of an amino acid sequence of SEQ ID NO: 26; a CDR2 comprising or consisting of an amino acid sequence of SEQ ID NO: 35; a CDR3 comprising or consisting of an amino acid sequence of SEQ ID NO: 45,
or
(g) a light chain variable domain (VL) comprising a complementarity determining region (CDR) 1 comprising or consisting of an amino acid sequence of SEQ ID NOs: 56; a CDR2 comprising or consisting of an amino acid sequence of SEQ ID NO: 63; a CDR3 comprising or consisting of an amino acid sequence of SEQ ID NO: 69; and
(h) a heavy chain variable domain (VH) comprising a complementarity determining region (CDR) 1 comprising or consisting of an amino acid sequence of SEQ ID NO: 28; a CDR2 comprising or consisting of an amino acid sequence of SEQ ID NO: 37; a CDR3 comprising or consisting of an amino acid sequence of SEQ ID NO: 46,
or
(i) a light chain variable domain (VL) comprising a complementarity determining region (CDR) 1 comprising or consisting of an amino acid sequence of SEQ ID NOs: 57; a CDR2 comprising or consisting of an amino acid sequence of SEQ ID NO: 64; a CDR3 comprising or consisting of an amino acid sequence of SEQ ID NO: 68; and
(j) a heavy chain variable domain (VH) comprising a complementarity determining region (CDR) 1 comprising or consisting of an amino acid sequence of SEQ ID NO: 29; a CDR2 comprising or consisting of an amino acid sequence of SEQ ID NO: 38; a CDR3 comprising or consisting of an amino acid sequence of SEQ ID NO: 47,
or
(k) a light chain variable domain (VL) comprising a complementarity determining region (CDR) 1 comprising or consisting of an amino acid sequence of SEQ ID NOs: 58; a CDR2 comprising or consisting of an amino acid sequence of SEQ ID NO: 62; a CDR3 comprising or consisting of an amino acid sequence of SEQ ID NO: 68; and
(l) a heavy chain variable domain (VH) comprising a complementarity determining region (CDR) 1 comprising or consisting of an amino acid sequence of SEQ ID NO: 27; a CDR2 comprising or consisting of an amino acid sequence of SEQ ID NO: 36; a CDR3 comprising or consisting of an amino acid sequence of SEQ ID NO: 48.

In one embodiment, the antigen binding domain comprises:
(aa) where the VL is defined in (a) according to any aspect or embodiment herein, the VL comprises a framework region (FR) 1 comprising or consisting of a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence set forth in SEQ ID NO: 51; a FR2 comprising or consisting of a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence set forth in SEQ ID NO: 59; a FR3 comprising or consisting of a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence set forth in SEQ ID NO: 65; and a FR4 comprising or consisting of a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence set forth in SEQ ID NO: 70, and
(bb) where the VH is defined in (b) according to any aspect or embodiment herein, the VH comprises a framework region (FR) 1 comprising or consisting of a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence set forth in SEQ ID NO: 23; a FR2 comprising or consisting of a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence set forth in SEQ ID NO: 31; a FR3 comprising or consisting of a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence set forth in SEQ ID NO: 41; and a FR4 comprising or consisting of a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence set forth in SEQ ID NO: 49,
or
(cc) where the VL is defined in (c) according to any aspect or embodiment herein, the VL comprises a framework region (FR) 1 comprising or consisting of a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence set forth in SEQ ID NO: 51; a FR2 comprising or consisting of a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence set forth in SEQ ID NO: 59; a FR3 comprising or consisting of a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence set forth in SEQ ID NO: 65; and a FR4 comprising or consisting of a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence set forth in SEQ ID NO: 70, and
(dd) where the VH is defined in (d) according to any aspect or embodiment herein, the VH comprises a framework region (FR) 1 comprising or consisting of a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence set forth in SEQ ID NO: 22; a FR2 comprising or consisting of a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence set forth in SEQ ID NO: 30; a FR3 comprising or consisting of a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence set forth in SEQ ID NO: 39; and a FR4 comprising or consisting of a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence set forth in SEQ ID NO: 49, or
(ee) where the VL is defined in (e) according to any aspect or embodiment herein, the VL comprises a framework region (FR) 1 comprising or consisting of a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence set forth in SEQ ID NO: 51; a FR2 comprising or consisting of a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence set forth in SEQ ID NO: 59; a FR3 comprising or consisting of a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence set forth in SEQ ID NO: 65; and a FR4 comprising or consisting of a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence set forth in SEQ ID NO: 70, and
(ff) where the VH is defined in (f) according to any aspect or embodiment herein, the VH comprises a framework region (FR) 1 comprising or consisting of a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence set forth in SEQ ID NO: 22; a FR2 comprising or consisting of a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence set forth in SEQ ID NO: 31; a FR3 comprising or consisting of a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence set forth in SEQ ID NO: 40; and a FR4 comprising or consisting of a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence set forth in SEQ ID NO: 49,
or
(gg) where the VL is defined in (g) according to any aspect or embodiment herein, the VL comprises a framework region (FR) 1 comprising or consisting of a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence set forth in SEQ ID NO: 52; a FR2 comprising or consisting of a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence set forth in SEQ ID NO: 60; a FR3 comprising or consisting of a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence set forth in SEQ ID NO: 66; and a FR4 comprising or consisting of a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence set forth in SEQ ID NO: 71, and
(hh) where the VH is defined in (h) according to any aspect or embodiment herein, the VH comprises a framework region (FR) 1 comprising or consisting of a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence set forth in SEQ ID NO: 24; a FR2 comprising or consisting of a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence set forth in SEQ ID NO: 32; a FR3 comprising or consisting of a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence set forth in SEQ ID NO: 42; and a FR4 comprising or consisting of a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence set forth in SEQ ID NO: 50,
or
(ii) where the VL is defined in (i) according to any aspect or embodiment herein, the VL comprises a framework region (FR) 1 comprising or consisting of a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence set forth in SEQ ID NO: 53; a FR2 comprising or consisting of a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence set forth in SEQ ID NO: 61; a FR3 comprising or consisting of a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence set forth in SEQ ID NO: 67; and a FR4 comprising or consisting of a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence set forth in SEQ ID NO: 70, and
(jj) where the VH is defined in (j) according to any aspect or embodiment herein, the VH comprises a framework region (FR) 1 comprising or consisting of a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence set forth in SEQ ID NO: 22; a FR2 comprising or consisting of a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence set forth in SEQ ID NO: 33; a FR3 comprising or consisting of a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence set forth in SEQ ID NO: 43; and a FR4 comprising or consisting of a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence set forth in SEQ ID NO: 49,
or
(kk) where the VL is defined in (k) according to any aspect or embodiment herein, the VL comprises a framework region (FR) 1 comprising or consisting of a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence set forth in SEQ ID NO: 54; a FR2 comprising or consisting of a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence set forth in SEQ ID NO: 59; a FR3 comprising or consisting of a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence set forth in SEQ ID NO: 65; and a FR4 comprising or consisting of a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence set forth in SEQ ID NO: 70, and
(ll) where the VH is defined in (l) according to any aspect or embodiment herein, the VH comprises a framework region (FR) 1 comprising or consisting of a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence set forth in SEQ ID NO: 23; a FR2 comprising or consisting of a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence set forth in SEQ ID NO: 31; a FR3 comprising or consisting of a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence set forth in SEQ ID NO: 44; and a FR4 comprising or consisting of a sequence at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence set forth in SEQ ID NO: 49.

In one embodiment, the antigen binding domain comprises:
(aa) where the VL is defined in (a) according to any aspect or embodiment herein, the VL comprises a framework region (FR) 1 comprising or consisting of an amino acid sequence of SEQ ID NO: 51; a FR2 comprising or consisting of an amino acid sequence of SEQ ID NO: 59; a FR3 comprising or consisting of an amino acid sequence of SEQ ID NO: 65; and a FR4 comprising or consisting of an amino acid sequence of SEQ ID NO: 70, and
(bb) where the VH is defined in (b) according to any aspect or embodiment herein, the VH comprises a framework region (FR) 1 comprising or consisting of an amino acid sequence of SEQ ID NO: 23; a FR2 comprising or consisting of an amino acid sequence of SEQ ID NO: 31; a FR3 comprising or consisting of an amino acid sequence of SEQ ID NO: 41; and a FR4 comprising or consisting of an amino acid sequence of SEQ ID NO: 49,
or
(cc) where the VL is defined in (c) according to any aspect or embodiment herein, the VL comprises a framework region (FR) 1 comprising or consisting of an amino acid sequence of SEQ ID NO: 51; a FR2 comprising or consisting of an amino acid sequence of SEQ ID NO: 59; a FR3 comprising or consisting of an amino acid sequence of SEQ ID NO: 65; and a FR4 comprising or consisting of an amino acid sequence of SEQ ID NO: 70, and
(dd) where the VH is defined in (d) according to any aspect or embodiment herein, the VH comprises a framework region (FR) 1 comprising or consisting of an amino acid sequence of SEQ ID NO: 22; a FR2 comprising or consisting of an amino acid sequence of SEQ ID NO: 30; a FR3 comprising or consisting of an amino acid sequence of SEQ ID NO: 39; and a FR4 comprising or consisting of an amino acid sequence of SEQ ID NO: 49,
or
(ee) where the VL is defined in (e) according to any aspect or embodiment herein, the VL comprises a framework region (FR) 1 comprising or consisting of an amino acid sequence of SEQ ID NO: 51; a FR2 comprising or consisting of an amino acid sequence of SEQ ID NO: 59; a FR3 comprising or consisting of an amino acid sequence of SEQ ID NO: 65; and a FR4 comprising or consisting of an amino acid sequence of SEQ ID NO: 70, and
(ff) where the VH is defined in (f) according to any aspect or embodiment herein, the VH comprises a framework region (FR) 1 comprising or consisting of an amino acid sequence of SEQ ID NO: 22; a FR2 comprising or consisting of an amino acid sequence of SEQ ID NO: 31; a FR3 comprising or consisting of an amino acid sequence of SEQ ID NO: 40; and a FR4 comprising or consisting of an amino acid sequence of SEQ ID NO: 49,
or
(gg) where the VL is defined in (g) according to any aspect or embodiment herein, the VL comprises a framework region (FR) 1 comprising or consisting of an amino acid sequence of SEQ ID NO: 52; a FR2 comprising or consisting of an amino acid sequence of SEQ ID NO: 60; a FR3 comprising or consisting of an amino acid sequence of SEQ ID NO: 66; and a FR4 comprising or consisting of an amino acid sequence of SEQ ID NO: 71, and
(hh) where the VH is defined in (h) according to any aspect or embodiment herein, the VH comprises a framework region (FR) 1 comprising or consisting of an amino acid sequence of SEQ ID NO: 24; a FR2 comprising or consisting of an amino acid sequence of SEQ ID NO: 32; a FR3 comprising or consisting of an amino acid sequence of SEQ ID NO: 42; and a FR4 comprising or consisting of an amino acid sequence of SEQ ID NO: 50,
or
(ii) where the VL is defined in (i) according to any aspect or embodiment herein, the VL comprises a framework region (FR) 1 comprising or consisting of an amino acid sequence of SEQ ID NO: 53; a FR2 comprising or consisting of an amino acid sequence of SEQ ID NO: 61; a FR3 comprising or consisting of an amino acid sequence of SEQ ID NO: 67; and a FR4 comprising or consisting of an amino acid sequence of SEQ ID NO: 70, and
(jj) where the VH is defined in (j) according to any aspect or embodiment herein, the VH comprises a framework region (FR) 1 comprising or consisting of an amino acid sequence of SEQ ID NO: 22; a FR2 comprising or consisting of an amino acid sequence of SEQ ID NO: 33; a FR3 comprising or consisting of an amino acid sequence of SEQ ID NO: 43; and a FR4 comprising or consisting of an amino acid sequence of SEQ ID NO: 49,
or
(kk) where the VL is defined in (k) according to any aspect or embodiment herein, the VL comprises a framework region (FR) 1 comprising or consisting of an amino acid sequence of SEQ ID NO: 54; a FR2 comprising or consisting of an amino acid sequence of SEQ ID NO: 59; a FR3 comprising or consisting of an amino acid sequence of SEQ ID NO: 65; and a FR4 comprising or consisting of an amino acid sequence of SEQ ID NO: 70, and
(ll) where the VH is defined in (l) according to any aspect or embodiment herein, the VH comprises a framework region (FR) 1 comprising or consisting of an amino acid sequence of SEQ ID NO: 23; a FR2 comprising or consisting of an amino acid sequence of SEQ ID NO: 31; a FR3 comprising or consisting of an amino acid sequence of SEQ ID NO: 44; and a FR4 comprising or consisting of an amino acid sequence of SEQ ID NO:

49.

In any aspect or embodiment, the antigen binding domain may be a single-chain variable fragment (scFv). As would be understood in the art, a scFv is a fusion protein comprising two portions that may share homology with, or may be identical to, the variable-heavy (VH) and variable-light (VL) chains of an antibody, with the two portions connected together with a linker peptide. For example, the scFv may include VH and VL amino acid sequences that are derived from an antibody that recognises mesothelin. In this context it will be appreciated that the term “derived from” is not a reference to the source of the polypeptides per se, but rather refers to the derivation of the amino acid sequence that constitute a portion of the antigen-binding region. Consequently, the term “derived from” includes synthetically, artificially or otherwise created polypeptides that share sequence identity to an antibody that binds to mesothelin.

In some embodiments, the antigen-recognition domain includes amino acid sequence homology to the amino acid sequence of a multivalent scFv. In some embodiments, the multivalent scFv is a divalent or trivalent scFv.

In some embodiments, the antigen-recognition domain has the amino acid sequence of a single-antibody domain (sdAb) that binds to mesothelin.

In aspect or embodiment, the CAR further comprises an epitope or affinity tag. Typically the epitope or affinity tag is C-terminal to the antigen binding domain. Preferably, the epitope or affinity tag is located between the antigen binding domain and the transmembrane domain.

The epitope or affinity tag may be a peptide tag or a protein tag. The peptide tag may be any one of FLAG-tag, HA-tag, His-tag, Myc-tag, S-tag, SBP-tag, Strep-tag or eXact-tag. The protein tag may be any one of GST-tag, MBP-tag or GFP-tag.

Preferably the epitope tag comprises or consists of an amino acid sequence of SEQ ID NO: 11.

In any aspect or embodiment, the CAR further comprises a hinge. Preferably the hinge is derived from CD8a. Typically the hinge is C-terminal to the antigen binding domain. In one embodiment, the hinge is located between the antigen binding domain and the transmembrane domain.

Preferably the hinge comprises or consists of an amino acid sequence of SEQ ID NO: 13 or 19.

In any aspect or embodiment, the transmembrane domain comprises a portion from CD8a. Preferably the transmembrane domain comprises or consists of the amino acid sequence of SEQ ID NO: 14 or 20.

In any aspect or embodiment, the signalling domain comprises a portion derived from an activation receptor. In some embodiments, the activation receptor is a member of the CD3 co-receptor complex. In some embodiments, the portion derived from the CD3 co-receptor complex is CD3-ζ (CD3-zeta). Preferably the signalling domain comprises an amino acid sequence of SEQ ID NO: 17 or 21.

In some embodiments, the signalling domain comprises a portion derived from a co-stimulatory receptor. In some embodiments, the co-stimulatory receptor is CD28 or 4-1 BB (CD137). Preferably the signalling domain comprises an amino acid sequence of SEQ ID NO: 15 or 16.

In some embodiments, the signalling domain comprises a portion derived from an activation receptor and a portion derived from a co-stimulatory receptor. In some embodiments, the activation receptor is a member of the CD3 co-receptor complex and the co-stimulatory receptor is selected CD28. In some embodiments, the activation receptor is a member of the CD3 co-receptor complex and the co-stimulatory receptor is CD28 or 4-1 BB (CD137). Preferably, the portion derived from the CD3 co-receptor complex is CD3-ζ (CD3-zeta). The signalling domain may comprises or consist of an amino acid sequence of SEQ ID Nos: 15 and 17; 15 and 21; 16 and 17; or 16 and 21.

In one embodiment, the CAR comprises (from N to C terminus) and antigen binding domain as described herein, a linker, an epitope or affinity tag (preferably a FLAG tag), CD8a hinge, a CD8a transmembrane domain, a CD28 signalling domain and a CD3ζ (zeta) signalling domain.

In one embodiment, the CAR comprises (from N to C terminus) and antigen binding domain as described herein, a linker, an epitope or affinity tag (preferably a FLAG tag), CD8a hinge, a CD8a transmembrane domain, a 4-1 BB signalling domain and a CD3ζ (zeta) signalling domain.

In one embodiment, the CAR comprises or consists of an amino acid sequence of SEQ ID NO: 7 or 8.

In another aspect, the present invention provides a nucleic acid molecule including a nucleotide sequence encoding the chimeric antigen receptor according to the invention described herein.

In one embodiment, the present invention provides nucleic acid comprising or consisting of a nucleotide sequence encoding a chimeric antigen receptor (CAR) comprising:
(i) an antigen recognition domain that recognises mesothelin;
(ii) a transmembrane domain; and
(iii) an intracellular signalling domain,
wherein the antigen recognition domain comprises:
(a) a light chain variable domain (VL) comprising three complementarity determining regions (CDRs) of the amino acid sequence shown in SEQ ID NO: 2; and
(b) a heavy chain variable domain (VH) comprising three complementarity determining regions (CDRs) of the amino acid sequence shown in SEQ ID NO: 1.

In one embodiment, the nucleic acid comprises:
(a) three nucleotides sequences within SEQ ID NO: 73 that encode three complementarity determining regions (CDRs) of the amino acid sequence shown in SEQ ID NO: 2; and
(b) three nucleotides sequences within SEQ ID NO: 72 that encode three complementarity determining regions (CDRs) of the amino acid sequence shown in SEQ ID NO: 1.

In one embodiment, the nucleic acid comprises or consists of a nucleotide sequence that encodes a chimeric antigen receptor (CAR) comprising:
(i) an antigen recognition domain that recognises mesothelin;
(ii) a transmembrane domain; and
(iii) an intracellular signalling domain,
wherein the nucleotide sequence comprises:
(a) SEQ ID NOs: 126, 127, 128, or 129; SEQ ID NO: 133, 134, or 135; and SEQ ID NO: 139 or 140; and
(b) SEQ ID NO: 96, 97, 98, 99 or 100; SEQ ID NO: 105, 106, 107, 108 or 109; and SEQ ID NO: 116, 117, 118 or 119.

In one embodiment, the nucleotide sequence encoding the antigen recognition domain comprises:
(a) SEQ ID NOs: 126; SEQ ID NO: 133; and SEQ ID NO: 139; and
(b) SEQ ID NOs: 98; SEQ ID NO: 107; and SEQ ID NO: 116,
or
(c) SEQ ID NOs: 126; SEQ ID NO: 133; and SEQ ID NO: 139; and
(d) SEQ ID NOs: 96; SEQ ID NO: 105; and SEQ ID NO: 116,
or
(e) SEQ ID NOs: 126; SEQ ID NO: 133; and SEQ ID NO: 139; and
(f) SEQ ID NOs: 97; SEQ ID NO: 106; and SEQ ID NO: 116,
or
(g) SEQ ID NOs: 127; SEQ ID NO: 134; and SEQ ID NO: 140; and
(h) SEQ ID NOs: 99; SEQ ID NO: 108; and SEQ ID NO: 117,
or
(i) SEQ ID NOs: 128; SEQ ID NO: 135; and SEQ ID NO: 139; and
(j) SEQ ID NOs: 100; SEQ ID NO: 109; and SEQ ID NO: 118,
or
(k) SEQ ID NOs: 129; SEQ ID NO: 133; and SEQ ID NO: 139; and
(l) SEQ ID NOs: 98; SEQ ID NO: 107; and SEQ ID NO: 119.

In one embodiment, nucleotide sequence encoding the antigen binding domain comprises:
(aa) where the nucleotide sequence comprises the sequences defined in (a) according to any aspect or embodiment herein, the nucleotide sequence further comprises SEQ ID NO: 122; SEQ ID NO: 130; SEQ ID NO: 136; and SEQ ID NO: 141, and
(bb) where the nucleotide sequence comprises the sequences defined in (b) according to any aspect or embodiment herein, the nucleotide sequence further comprises SEQ ID NO: 94; SEQ ID NO: 102; SEQ ID NO: 112; and SEQ ID NO: 120,
or
(cc) where the nucleotide sequence comprises the sequences defined in (c) according to any aspect or embodiment herein, the nucleotide sequence further comprises SEQ ID NO: 122; SEQ ID NO: 130; SEQ ID NO: 136; and SEQ ID NO: 141, and
(dd) where the nucleotide sequence comprises the sequences defined in (d) according to any aspect or embodiment herein, the nucleotide sequence further comprises SEQ ID NO: 93; SEQ ID NO: 101; SEQ ID NO: 110; and SEQ ID NO: 120,
or
(ee) where the nucleotide sequence comprises the sequences defined in (e) according to any aspect or embodiment herein, the nucleotide sequence further comprises SEQ ID NO: 122; SEQ ID NO: 130; SEQ ID NO: 136; and SEQ ID NO: 141, and
(ff) where the nucleotide sequence comprises the sequences defined in (f) according to any aspect or embodiment herein, the nucleotide sequence further comprises SEQ ID NO: 93; SEQ ID NO: 102; SEQ ID NO: 111; and SEQ ID NO: 120,
or
(gg) where the nucleotide sequence comprises the sequences defined in (g) according to any aspect or embodiment herein, the nucleotide sequence further comprises SEQ ID NO: 123; SEQ ID NO: 131; SEQ ID NO: 137; and SEQ ID NO: 142, and
(hh) where the nucleotide sequence comprises the sequences defined in (h) according to any aspect or embodiment herein, the nucleotide sequence further comprises SEQ ID NO: 95; SEQ ID NO: 103; SEQ ID NO: 113; and SEQ ID NO: 121, or
(ii) where the nucleotide sequence comprises the sequences defined in (i) according to any aspect or embodiment herein, the nucleotide sequence further comprises SEQ ID NO: 124; SEQ ID NO: 132; SEQ ID NO: 138; and SEQ ID NO: 141, and
(jj) where the nucleotide sequence comprises the sequences defined in (j) according to any aspect or embodiment herein, the nucleotide sequence further comprises SEQ ID NO: 93; SEQ ID NO: 104; SEQ ID NO: 114; and SEQ ID NO: 120,
or
(kk) where the nucleotide sequence comprises the sequences defined in (k) according to any aspect or embodiment herein, the nucleotide sequence further comprises SEQ ID NO: 125; SEQ ID NO: 130; SEQ ID NO: 136; and SEQ ID NO: 141, and
(l) where the nucleotide sequence comprises the sequences defined in (l) according to any aspect or embodiment herein, the nucleotide sequence further comprises SEQ ID NO: 94; SEQ ID NO: 102; SEQ ID NO: 115; and SEQ ID NO: 120.

In any embodiment, the nucleotide sequence encoding the antigen binding domain comprises or consist of SEQ ID NO: 72 and 73; 74; 75; or 76.

In any embodiment, the nucleic acid further comprises a nucleotide sequence encoding:
(a) a linker, preferably the nucleotide sequence encoding a linker comprises or consists of SEQ ID NO: 80 or 81;
(b) an affinity tag, preferably the nucleotide sequence encoding an affinity tag comprises or consist of SEQ ID NO: 82, or a nucleotide sequence encoding any one of SEQ ID NO:11, 143, 144, 145, 146, 147, 148, 149, 150, 151, 152, 153 or 154;
(c) a hinge, preferably the nucleotide sequence encoding a hinge comprises or consists of SEQ ID NO: 83, 84, 89 or 90, or any human allelic variants thereof,
(d) a transmembrane domain, preferably the nucleotide sequence encoding a transmembrane domain comprises or consists of SEQ ID NO: 83, 85, 89 or 91, or any human allelic variants thereof; and/or
(e) a signalling domain, preferably the nucleotide sequence coding a signalling domain comprises or consist of SEQ ID NO: 86, 87, 88 or 92; or SEQ ID NO: 86 and 88; or SEQ ID NO: 86 and 92; or SEQ ID NO: 87 and 88; or SEQ ID NO: 87 and 92, or any human allelic variants thereof.

In any embodiment, the nucleic acid comprises or consists of SEQ ID NO: 77, 78, 79 or 156.

In another aspect, the present invention provides a nucleic acid construct that includes a nucleic acid molecule according to the invention described herein. In some embodiments, expression of the nucleic acid molecule is under the control of a transcriptional control sequence. In some embodiments, the transcriptional control sequence may be a constitutive promoter or an inducible promoter.

In some embodiments, the nucleic acid construct further includes an internal ribosome entry site (IRES) that allows for translation initiation within the mRNA once expressed from the nucleic acid construct.

In some embodiments, the nucleic acid construct is a vector such as a viral vector, which can be used to transform an immune cell, for example a T cell, to induce expression of the CAR.

In another aspect, the present invention provides a genetically modified cell that comprises a CAR according to the invention described herein.

In another aspect, the present invention provides a genetically modified cell that comprises a nucleic acid molecule according to the invention described herein, or a nucleic acid construct of the invention described herein, or a genomically integrated form of the construct.

In another aspect, the present invention provides a method of generating a genetically modified cell, said method comprising transducing the cell, preferably an immune cell, with a nucleic acid construct encoding a CAR of the invention as described herein, so that the transduced cell expresses the CAR, thereby generating a genetically modified cell.

In some embodiments, the cell is an immune cell such as a leukocyte. In some embodiments, the cell is a Peripheral Blood Mononuclear Cell (PBMC), a lymphocyte, a T cell (including a CD4+ T cell or a CD8+ T cell), a natural killer (NK) cell, a natural killer T cell or a tumour infiltrating lymphocyte (TIL).

In a preferred embodiment, the immune cell that expresses the CAR is a T cell. Illustrative examples of suitable T cells include helper T cells (HTL; CD4⁺ T cell), a cytotoxic T cell (CTL; CD8⁺ T cell), CD4⁺CD8⁺ T cell, CD4⁻CD8⁻ T cell, or any other subset of T cells. Other illustrative examples of suitable T cells include T cells expressing one or more of the following markers: CD3, CD4, CD8, CD27, CD28, CD45RA, CD45RO, CD62L, CD127, CD197, and HLA-DR.

In another aspect, the present invention provides a method of generating a CAR-T cell, the method comprising transducing a T cell with a nucleic acid construct encoding a CAR of the invention as described herein so that the transduced T cell expresses the CAR, thereby generating a CAR-T cell.

In another aspect, the present invention provides a method of killing a cell expressing mesothelin, the method comprising exposing the cell expressing mesothelin to a genetically modified cell of the invention described herein, thereby killing a cell expressing mesothelin.

In some embodiments, the cell expressing mesothelin is a cancer cell. In some embodiments the cancer is selected from the group consisting of; brain cancer, oesophageal cancer, mouth cancer, tongue cancer, thyroid cancer, lung cancer, stomach cancer, pancreatic cancer, kidney cancer, colon cancer, rectal cancer, prostate cancer, bladder cancer, cervical cancer, epithelial cell cancers, skin cancer, leukaemia, lymphoma, myeloma, breast cancer, ovarian cancer, endometrial cancer and testicular cancer. In some embodiments the cancer is selected from the group consisting of; lung cancer, oesophageal cancer, stomach cancer, colon cancer, prostate cancer, bladder cancer, cervical cancer, vaginal cancers, epithelial cell cancers, skin cancer, blood-related cancers, breast cancer, endometrial cancer, uterine cancer and testicular cancer.

In some embodiments, the cancer is metastatic. In some embodiments, the cancer is stage III cancer or is stage IV cancer.

In another aspect, the present invention provides a method of expanding in vitro the genetically modified cell of the invention described herein, the method comprising the step of exposing the cell to an antigen for the CAR. In some embodiments, the method includes the further step of exposing the cell to a cytokine.

In another aspect, the present invention provides a method of expanding in vitro the genetically modified cell of the invention described herein, the method comprising the step of exposing the cell to an antigen for the CAR, e.g. mesothelin, and simultaneously exposing the cell to a cytokine.

In some embodiments, the cytokine is a member of the IL-2 subfamily, the interferon subfamily, the IL-10 subfamily, the IL-1 subfamily, the IL-17 subfamily or the TGF-β subfamily.

In some embodiments, the cytokine is selected from the group consisting of IFN-γ, IL-2, IL-5, IL-7, IL-8, IL-10, IL-12, IL-13, IL-15, IL-17, IL-18, TNF-α, TGF-β1, TGF-β2, TGF-β3 and GM-CSF, or a combination thereof.

In another aspect, the present invention provides a method of expanding in vitro the genetically modified cell of the invention described herein, the method comprising
exposing the cell to immobilised CD3 and CD28 agonists; and
contacting the cells with a media under conditions that allow proliferation of the cells, preferably T cells, preferably human T cells.

In some embodiments, the agonists are immobilised on a beaded substrate. In one embodiment, the agonists may be immobilised on “Human Activator” Dynabeads™. In another preferred example, the agonists are immobilised on a colloidal polymeric nanomatrix beaded substrate conjugated to recombinant humanized CD3 and CD28 agonist (for example on a “MACS GMP” TransAct™ beaded substrate).

In some embodiments the CD3 and CD28 agonists are anti-CD3 and anti-CD28 antibodies.

In some embodiments the media is TexMACS™ GMP media. In some embodiments the media supplemented with interleukins, for example IL-7 and IL-15.

In some embodiments, the antibodies are immobilised on a surface of a tissue culture vessel such as a surface of a culture flask, plate or bioreactor.

In another aspect, the present invention provides a pharmaceutical composition including a genetically modified cell of the invention described herein and a pharmaceutically acceptable carrier. In some embodiments, the pharmaceutical composition comprises suitable adjuvants which may consist of cytokines. In some embodiments, the pharmaceutical composition may also comprise an intermediate as described herein.

In another aspect, the present invention provides a method of:
treating, preventing or minimising progression of cancer in a subject,
minimising, reducing or preventing growth of a tumour in a subject,
minimising, reducing or preventing metastasis in a subject, or
increasing survival of a subject,
   the method comprising, administering to the subject a CAR of the invention described herein, a nucleic acid construct of the invention as described herein, a genetically modified cell of the invention as described herein, or a pharmaceutical composition of the invention as described herein, thereby:
treating, preventing or minimising progression of cancer in a subject,
minimising, reducing or preventing growth of a tumour in a subject,
minimising, reducing or preventing metastasis in a subject, or
increasing survival of a subject,
In another aspect, the present invention provides use of a CAR of the invention described herein, a nucleic acid construct of the invention as described herein, a genetically modified cell of the invention as described herein, or a pharmaceutical composition of the invention as described herein, in the manufacture of a medicament for:
treating, preventing or minimising progression of cancer in a subject,
minimising, reducing or preventing growth of a tumour in a subject,
minimising, reducing or preventing metastasis in a subject, or
increasing survival of a subject.

In another aspect, the present invention provides a CAR of the invention described herein, a nucleic acid construct of the invention as described herein, a genetically modified cell of the invention as described herein, or a pharmaceutical composition of the invention as described herein for use in:
treating, preventing or minimising progression of cancer in a subject,
minimising, reducing or preventing growth of a tumour in a subject,
minimising, reducing or preventing metastasis in a subject, or
increasing survival of a subject.

As used herein, except where the context requires otherwise, the term “comprise” and variations of the term, such as “comprising”, “comprises” and “comprised”, are not intended to exclude further additives, components, integers or steps.

Further aspects of the present invention and further embodiments of the aspects described in the preceding paragraphs will become apparent from the following description, given by way of example and with reference to the accompanying drawings.

### BRIEF DESCRIPTION OF THE DRAWINGS

FIG. 1. Sensorgrams of Mesothelin binding to 7B1 IgG1 LALA AA cAb7385-10.16 on FC3-1 (210805 experiment). Mesothelin concentrations 111 nM, 37.0 nM, 12.3 nM, 4.1 nM, 1.4 nM, 0.46 nM, 0.15 nM from top to bottom.

FIG. 2. Schematic of 7B1-TAG-CD28-CD3zeta and 7B1-TAG-41BB-CD3zeta CAR designs.

FIG. 3. 7B1 CAR T cells bind to anti-FLAG and recombinant human mesothelin.

FIG. 4. Anti-MSLN (7B1) CAR T cell assessment with soluble mesothelin. A dose response of soluble mesothelin (1-100 ng/mL) was added in combination with the anti-mesothelin CAR 7B1-28z and mesothelin negative cells (SKOV3 and A549) and positive cells with various levels of mesothelin (SKOV3-MSLN, ASPC-1, HGC-27)

FIGS. 5A-5E. Cytokine analysis of 7B1 CAR T cells. 7B1 CD28z and 41 BB transduced T-cells were incubated with recombinant proteins (anti-CD3, anti-FLAG, mesothelin) and tumour cell lines (negative, ASPC1 and OVCAR3 mesothelin positive for 24 hours before a panel of cytokines was measured.

FIG. 6. Cytokine analysis with various antigen density (tumour cell lines). Cell lines with varying mesothelin expression were incubated with 7B1-28z CAR T-cells for 24 hours. Cytokines were measured by LegendPlex cytokine bead array.

FIGS. 7A-7B. CAR T-cell proliferation. Cell lines with varying mesothelin expression (ASPC1 pancreatic and SKOV3-mesothelin transfected) were incubated with 7B1-28z CAR T-cells.

FIG. 8. Variation of cell phenotypes seen between donors.

FIGS. 9A-9B. 7B1 CAR transduction in patient and healthy donor cells. Apheresis and healthy buffy pack PBMC were activated in Transact and IL-7/15. On D2 patient apheresis and healthy donor PBMC were transduced with 7B1-28z lentivirus at M01=2. Cells were expanded for 4 days with TexMACS media and IL-7/15 and then analyzed for CAR expression on D7

FIG. 10. Phenotypes of patient and healthy donor CAR T cells.

FIG. 11. Function of patient and healthy donor CART cells. 100 k CART were cultured in a 1:1 culture with ASPC-1 (mesothelin positive) or MDA-MB-468 (mesothelin negative) cells, or plate bound FLAG/CD3 for 24 hours. Supernatants were then analysed for IFNg secretion using Perkin Elmer AlphaLISA kit.

FIGS. 12A-12D. Cytotoxicity analysis of CAR T cells. (FIG. 12A) Cytolytic capacity of 7B1-28z was confirmed using a luciferase assay (24 h) with the SKOV-3 MESO transfected cell line in a dose response manner. CD40 was depleted in the parental and MSLN-expressing cell line to reduce the background cell killing that was not mesothelin-dependent. (FIG. 12B) 7B1-28z cytotoxicity was confirmed using a luciferase assay (24 h) with the SKOV-3 MESO-CD40 knock out transfected cell line in a dose response manner as well as with the OVCAR-3 (ovarian cancer cell line +ve for mesothelin) compared to mesothelin negative cell lines MDA-MB-468 and SKOV CD40 KO. (FIGS. 12C-12D) 7B1-28z cytotoxicity was further confirmed using a 51Cr release assay against tumour cells expressing mesothelin after 24 hours at various effector to target ratios.

FIG. 13. Immunogenicity of 7B1-CD28z for DRB1 HLA class II epitopes.

FIG. 14. In vivo dose response of 7B1 CAR T cells. (FIG. 14A) Confirmation of mesothelin expression of SKOV-3 CD40 MSLN cells used in NSG model compared to negative cell line SKOV-3 CD40 KO. (FIG. 14B) Tumours established (50 mm 3) in NSG mice using SKOV3-Mesothelin transfected cells with CD40 knocked out. A single dose of 7B1-CD28z CAR was evaluated at three different concentrations (1-, 3-, 5-million cells) and body weight measured (FIG. 14C).

FIG. 15. Plasmid map of 7B1-28z-pCDH-EF1a.

### DESCRIPTION OF SEQUENCES

TABLE 1
Sequences of the invention

| SEQ ID | | |
|---|---|---|
| NO | Description | Sequence |
| 1 | 7B1 (anti-mesothelin | EVHLVESGGGLVQPGGSLRLSCAASGFTFSRYWMSWVRQAQGKGLE |
| | antibody)-VH | WVASIKQAGSEKTYVDSVKGRFTISRDNAKNSLSLQMNSLRAEDTAVYY |
| | | CAREGAYYYDSASYYPYYYYYSMDVWGQGTTVTVSS |
| 2 | 7B1 (anti-mesothelin | EIVLTQSPGTLSLSPGERATLSCRASQSVSSSYLAWYQQKPGQAPRLLI |
| | antibody)-VL | YGASSRATGIPDRFSGSGSGTDFTLTISRLEPEDFAVYYCQQYGSSQYT |
| | | FGQGTKLEIK |
| 3 | 7B1 scFv (VH-linker- | EVHLVESGGGLVQPGGSLRLSCAASGFTFSRYWMSWVRQAQGKGLE |
| | VL-HIS) | WVASIKQAGSEKTYVDSVKGRFTISRDNAKNSLSLQMNSLRAEDTAVYY |
| | | CAREGAYYYDSASYYPYYYYYSMDVWGQGTTVTVSSGGGGSGGGGS |
| | | GGGGSEIVLTQSPGTLSLSPGERATLSCRASQSVSSSYLAWYQQKPGQ |
| | | APRLLIYGASSRATGIPDRFSGSGSGTDFTLTISRLEPEDFAVYYCQQYG |
| | | SSQYTFGQGTKLEIKHHHHHHHHHH |
| 4 | 7B1 scFv (VH-linker- | EVHLVESGGGLVQPGGSLRLSCAASGFTFSRYWMSWVRQAQGKGLE |
| | VL)-without HIS | WVASIKQAGSEKTYVDSVKGRFTISRDNAKNSLSLQMNSLRAEDTAVYY |
| | | CAREGAYYYDSASYYPYYYYYSMDVWGQGTTVTVSSGGGGSGGGGS |
| | | GGGGSEIVLTQSPGTLSLSPGERATLSCRASQSVSSSYLAWYQQKPGQ |
| | | APRLLIYGASSRATGIPDRFSGSGSGTDFTLTISRLEPEDFAVYYCQQYG |
| | | SSQYTFGQGTKLEIK |
| 5 | 7B1 scFv (VH-linker- | MGWSCIILFLVATATGVHSEVHLVESGGGLVQPGGSLRLSCAASGFTFS |
| | VL-HIS) with leader | RYWMSWVRQAQGKGLEWVASIKQAGSEKTYVDSVKGRFTISRDNAKN |
| | sequence | SLSLQMNSLRAEDTAVYYCAREGAYYYDSASYYPYYYYYSMDVWGQG |
| | | TTVTVSSGGGGSGGGGSGGGGSGGGGSEIVLTQSPGTLSLSPGERAT |
| | | LSCRASQSVSSSYLAWYQQKPGQAPRLLIYGASSRATGIPDRFSGSGS |
| | | GTDFTLTISRLEPEDFAVYYCQQYGSSQYTFGQGTKLEIKHHHHHHHHH |
| | | H |
| 6 | 7B1 (anti-mesothelin | EVHLVESGGGLVQPGGSLRLSCAASGFTFSRYWMSWVRQAQGKGLE |
| | antibody) | WVASIKQAGSEKTYVDSVKGRFTISRDNAKNSLSLQMNSLRAEDTAVYY |
| | CAR extracellular | CAREGAYYYDSASYYPYYYYYSMDVWGQGTTVTVSSGGGGSGGGGS |
| | domain | GGGGSEIVLTQSPGTLSLSPGERATLSCRASQSVSSSYLAWYQQKPGQ |
| | | APRLLIYGASSRATGIPDRFSGSGSGTDFTLTISRLEPEDFAVYYCQQYG |
| | | SSQYTFGQGTKLEIKGGGGSDYKDDDDKLSNSIMYFSHFVPVFLPAKPT |
| | | TTPAPRPPTPAPTIASQPLSLRPEACRPAAGGAVHTRGLDFACD |
| 7 | 7B1-CD28z | EVHLVESGGGLVQPGGSLRLSCAASGFTFSRYWMSWVRQAQGKGLE |
| | | WVASIKQAGSEKTYVDSVKGRFTISRDNAKNSLSLQMNSLRAEDTAVYY |
| | | CAREGAYYYDSASYYPYYYYYSMDVWGQGTTVTVSSGGGGSGGGGS |
| | | GGGGSEIVLTQSPGTLSLSPGERATLSCRASQSVSSSYLAWYQQKPGQ |
| | | APRLLIYGASSRATGIPDRFSGSGSGTDFTLTISRLEPEDFAVYYCQQYG |
| | | SSQYTFGQGTKLEIKGGGGSDYKDDDDKLSNSIMYFSHFVPVFLPAKPT |
| | | TTPAPRPPTPAPTIASQPLSLRPEACRPAAGGAVHTRGLDFACDIYIWAP |
| | | LAGTCGVLLLSLVITLYCNHRNRSKRSRLLHSDYMNMTPRRPGPTRKHY |
| | | QPYAPPRDFAAYRSLRVKFSRSADAPAYQQGQNQLYNELNLGRREEY |
| | | DVLDKRRGRDPEMGGKPRRKNPQEGLYNELQKDKMAEAYSEIGMKGE |
| | | RRRGKGHDGLYQGLSTATKDTYDALHMQALPPR |
| 8 | 7B1-41BBz | EVHLVESGGGLVQPGGSLRLSCAASGFTFSRYWMSWVRQAQGKGLE |
| | | WVASIKQAGSEKTYVDSVKGRFTISRDNAKNSLSLQMNSLRAEDTAVYY |
| | | CAREGAYYYDSASYYPYYYYYSMDVWGQGTTVTVSSGGGGSGGGGS |
| | | GGGGSEIVLTQSPGTLSLSPGERATLSCRASQSVSSSYLAWYQQKPGQ |
| | | APRLLIYGASSRATGIPDRFSGSGSGTDFTLTISRLEPEDFAVYYCQQYG |
| | | SSQYTFGQGTKLEIKGGGGSDYKDDDDKLSNSIMYFSHFVPVFLPAKPT |
| | | TTPAPRPPTPAPTIASQPLSLRPEACRPAAGGAVHTRGLDFACDIYIWAP |
| | | LAGTCGVLLLSLVITLYCNHRNKRGRKKLLYIFKQPFMRPVQTTQEEDG |
| | | CSCRFPEEEEGGCELRVKFSRSADAPAYQQGQNQLYNELNLGRREEY |
| | | DVLDKRRGRDPEMGGKPRRKNPQEGLYNELQKDKMAEAYSEIGMKGE |
| | | RRRGKGHDGLYQGLSTATKDTYDALHMQALPPR |
| 9 | Linker | GGGGSGGGGSGGGGS |
| 10 | Linker variant | GGGGS |
| 11 | FLAG-tag octapeptide | DYKDDDDK |
| 12 | CD8a | LSNSIMYFSHFVPVFLPAKPTTTPAPRPPTPAPTIASQPLSLRPEACRPA |
| | | AGGAVHTRGLDFACDIYIWAPLAGTCGVLLLSLVITLYCNHRN |
| 13 | CD8a hinge | LSNSIMYFSHFVPVFLPAKPTTTPAPRPPTPAPTIASQPLSLRPEACRPA |
| | | AGGAVHTRGLDFACD |
| 14 | CD8a transmembrane | IYIWAPLAGTCGVLLLSLVITLYCNHRN |
| 15 | CD28 | RSKRSRLLHSDYMNMTPRRPGPTRKHYQPYAPPRDFAAYRS |
| 16 | 4-1BB | KRGRKKLLYIFKQPFMRPVQTTQEEDGCSCRFPEEEEGGCE |
| 17 | CD3zeta | RVKFSRSADAPAYQQGQNQLYNELNLGRREEYDVLDKRRGRDPEMG |
| | | GKPRRKNPQEGLYNELQKDKMAEAYSEIGMKGERRRGKGHDGLYQGL |
| | | STATKDTYDALHMQALPPR |
| 18 | CD8a-variant | TTTPAPRPPTPAPTIASQPLSLRPEACRPAAGGAVHTRGLDFACDIYIWA |
| | | PLAGTCGVLLLSLVITLYCNHRN |
| 19 | CD8a hinge-variant | TTTPAPRPPTPAPTIASQPLSLRPEACRPAAGGAVHTRGLDFACD |
| 20 | CD8a transmembrane- | IYIWAPLAGTCGVLLLSLVITLYC |
| | variant | |
| 21 | CD3zeta-variant | LRVKFSRSADAPAYQQGQNQLYNELNLGRREEYDVLDKRRGRDPEMG |
| | | GKPRRKNPQEGLYNELQKDKMAEAYSEIGMKGERRRGKGHDGLYQGL |
| | | STATKDTYDALHMQALPPR |
| 22 | HFR1 (Chothia, AbM, | EVHLVESGGGLVQPGGSLRLSCAAS |
| | IMGT) | |
| 23 | HFR1 (Kabat, Other) | EVHLVESGGGLVQPGGSLRLSCAASGFTFS |
| 24 | HFR1 (Contact) | EVHLVESGGGLVQPGGSLRLSCAASGFTF |
| 25 | CDR-H1 (Chothia) | GFTFSRY |
| 26 | CDR-H1 (AbM) | GFTFSRYWMS |
| 27 | CDR-H1 (Kabat, | RYWMS |
| | Other) | |
| 28 | CDR-H1 (Contact) | SRYWMS |
| 29 | CDR-H1 (IMGT) | GFTFSRYW |
| 30 | HFR2 (Chothia) | WMSWVRQAQGKGLEWVASI |
| 31 | HFR2 (AbM, Kabat, | WVRQAQGKGLEWVA |
| | Other) | |
| 32 | HFR2 (Contact) | WVRQAQGKGLE |
| 33 | HFR2 (IMGT) | MSWVRQAQGKGLEWVAS |
| 34 | CDR-H2 (Chothia) | KQAGSE |
| 35 | CDR-H2 (AbM) | SIKQAGSEKT |
| 36 | CDR-H2 (Kabat, | SIKQAGSEKTYVDSVKG |
| | Other) | |
| 37 | CDR-H2 (Contact) | WVASIKQAGSEKT |
| 38 | CDR-H2 (IMGT) | IKQAGSEK |
| 39 | HFR3 (Chothia) | KTYVDSVKGRFTISRDNAKNSLSLQMNSLRAEDTAVYYCAR |
| 40 | HFR3 (AbM) | YVDSVKGRFTISRDNAKNSLSLQMNSLRAEDTAVYYCAR |
| 41 | HFR3 (Kabat) | RFTISRDNAKNSLSLQMNSLRAEDTAVYYCAR |
| 42 | HFR3 (Contact) | YVDSVKGRFTISRDNAKNSLSLQMNSLRAEDTAVYYC |
| 43 | HFR3 (IMGT) | TYVDSVKGRFTISRDNAKNSLSLQMNSLRAEDTAVYYC |
| 44 | HFR3 (Other) | RFTISRDNAKNSLSLQMNSLRAED |
| 45 | CDR-H3 (Chothia, | EGAYYYDSASYYPYYYYYSMDV |
| | AbM, Kabat) | |
| 46 | CDR-H3 (Contact) | AREGAYYYDSASYYPYYYYYSMD |
| 47 | CDR-H3 (IMGT) | AREGAYYYDSASYYPYYYYYSMDV |
| 48 | CDR-H3 (Other) | TAVYYCAREGAYYYDSASYYPYYYYYSMDV |
| 49 | HFR4 (Chothia, AbM, | WGQGTTVTVSS |
| | Kabat, IMGT, Other) | |
| 50 | HFR4 (Contact) | VWGQGTTVTVSS |
| 51 | LFR1 (Chothia, AbM, | EIVLTQSPGTLSLSPGERATLSC |
| | Kabat) | |
| 52 | LFR1 (Contact) | EIVLTQSPGTLSLSPGERATLSCRASQSV |
| 53 | LFR1 (IMGT) | EIVLTQSPGTLSLSPGERATLSCRAS |
| 54 | LFR1 (Other) | EIVLTQSPGTLSLSPGERAT |
| 55 | CDR-L1 (Chothia, | RASQSVSSSYLA |
| | AbM, Kabat) | |
| 56 | CDR-L1 (Contact) | SSSYLAWY |
| 57 | CDR-L1 (IMGT) | QSVSSSY |
| 58 | CDR-L1 (Other) | LSCRASQSVSSSYLA |
| 59 | LFR2 (Chothia, AbM, | WYQQKPGQAPRLLIY |
| | Kabat, Other) | |
| 60 | LFR2 (Contact) | QQKPGQAPR |
| 61 | LFR2 (IMGT) | LAWYQQKPGQAPRLLIY |
| 62 | CDR-L2 (Chothia, | GASSRAT |
| | AbM, Kabat, Other) | |
| 63 | CDR-L2 (Contact) | LLIYGASSRA |
| 64 | CDR-L2 (IMGT) | GA |
| 65 | LFR3 (Chothia, AbM, | GIPDRFSGSGSGTDFTLTISRLEPEDFAVYYC |
| | Kabat, Other) | |
| 66 | LFR3 (Contact) | TGIPDRFSGSGSGTDFTLTISRLEPEDFAVYYC |
| 67 | LFR3 (IMGT) | SSRATGIPDRFSGSGSGTDFTLTISRLEPEDFAVYYC |
| 68 | CDR-L3 (Chothia, | QQYGSSQYT |
| | AbM, Kabat, IMGT, | |
| | Other) | |
| 69 | CDR-L3 (Contact) | QQYGSSQY |
| 70 | LFR4 (Chothia, AbM, | FGQGTKLEIK |
| | Kabat, IMGT, Other) | |
| 71 | LFR4 (Contact) | TFGQGTKLEIK |
| 72 | 7B1 (anti-mesothelin | GAGGTGCACCTGGTGGAGAGTGGTGGCGGTCTGGTGCAGCCAGGT |
| | antibody)-VH | GGTAGTCTGAGGCTGAGCTGCGCAGCAAGCGGCTTCACCTTCAGCA |
| | | GGTACTGGATGAGCTGGGTGAGGCAGGCCCAGGGCAAGGGCCTGG |
| | | AGTGGGTGGCCAGCATCAAGCAGGCCGGCAGCGAGAAGACCTACG |
| | | TGGACAGCGTGAAGGGCAGGTTCACCATCAGCAGGGACAACGCCAA |
| | | GAACAGCCTGAGCCTGCAGATGAACAGCCTGAGGGCCGAGGACAC |
| | | CGCCGTGTACTACTGCGCCAGGGAGGGCGCCTACTACTACGACAGC |
| | | GCCAGCTACTACCCCTACTACTACTACTACAGCATGGACGTGTGGG |
| | | GCCAGGGCACCACCGTGACCGTGAGCAGC |
| 73 | 7B1 (anti-mesothelin | GAGATCGTGCTGACCCAGAGCCCCGGCACCCTGAGCCTGAGCCCC |
| | antibody)-VL | GGCGAGAGGGCCACCCTGAGCTGCAGGGCCAGCCAGAGCGTGAGC |
| | | AGCAGCTACCTGGCCTGGTACCAGCAGAAGCCCGGCCAGGCCCCC |
| | | AGGCTGCTGATCTACGGCGCCAGCAGCAGGGCCACCGGCATCCCC |
| | | GACAGGTTCAGCGGCAGCGGCAGCGGCACCGACTTCACCCTGACC |
| | | ATCAGCAGGCTGGAGCCCGAGGACTTCGCCGTGTACTACTGCCAGC |
| | | AGTACGGCAGCAGCCAGTACACCTTCGGCCAGGGCACCAAGCTGG |
| | | AGATCAAG |
| 74 | 7B1 scFv (VH-linker- | GAGGTGCACCTGGTGGAGAGTGGTGGCGGTCTGGTGCAGCCAGGT |
| | VL-HIS) | GGTAGTCTGAGGCTGAGCTGCGCAGCAAGCGGCTTCACCTTCAGCA |
| | | GGTACTGGATGAGCTGGGTGAGGCAGGCCCAGGGCAAGGGCCTGG |
| | | AGTGGGTGGCCAGCATCAAGCAGGCCGGCAGCGAGAAGACCTACG |
| | | TGGACAGCGTGAAGGGCAGGTTCACCATCAGCAGGGACAACGCCAA |
| | | GAACAGCCTGAGCCTGCAGATGAACAGCCTGAGGGCCGAGGACAC |
| | | CGCCGTGTACTACTGCGCCAGGGAGGGCGCCTACTACTACGACAGC |
| | | GCCAGCTACTACCCCTACTACTACTACTACAGCATGGACGTGTGGG |
| | | GCCAGGGCACCACCGTGACCGTGAGCAGCGGCGGTGGCGGAAGC |
| | | GGTGGTGGCGGTAGCGGAGGTGGCGGCAGCGAGATCGTGCTGACC |
| | | CAGAGCCCCGGCACCCTGAGCCTGAGCCCCGGCGAGAGGGCCACC |
| | | CTGAGCTGCAGGGCCAGCCAGAGCGTGAGCAGCAGCTACCTGGCC |
| | | TGGTACCAGCAGAAGCCCGGCCAGGCCCCCAGGCTGCTGATCTAC |
| | | GGCGCCAGCAGCAGGGCCACCGGCATCCCCGACAGGTTCAGCGGC |
| | | AGCGGCAGCGGCACCGACTTCACCCTGACCATCAGCAGGCTGGAG |
| | | CCCGAGGACTTCGCCGTGTACTACTGCCAGCAGTACGGCAGCAGCC |
| | | AGTACACCTTCGGCCAGGGCACCAAGCTGGAGATCAAGCACCACCA |
| | | CCACCACCACCACCACCACCAC |
| 75 | 7B1 scFv (VH-linker- | GAGGTGCACCTGGTGGAGAGTGGTGGCGGTCTGGTGCAGCCAGGT |
| | VL)-without HIS | GGTAGTCTGAGGCTGAGCTGCGCAGCAAGCGGCTTCACCTTCAGCA |
| | | GGTACTGGATGAGCTGGGTGAGGCAGGCCCAGGGCAAGGGCCTGG |
| | | AGTGGGTGGCCAGCATCAAGCAGGCCGGCAGCGAGAAGACCTACG |
| | | TGGACAGCGTGAAGGGCAGGTTCACCATCAGCAGGGACAACGCCAA |
| | | GAACAGCCTGAGCCTGCAGATGAACAGCCTGAGGGCCGAGGACAC |
| | | CGCCGTGTACTACTGCGCCAGGGAGGGCGCCTACTACTACGACAGC |
| | | GCCAGCTACTACCCCTACTACTACTACTACAGCATGGACGTGTGGG |
| | | GCCAGGGCACCACCGTGACCGTGAGCAGCGGCGGTGGCGGAAGC |
| | | GGTGGTGGCGGTAGCGGAGGTGGCGGCAGCGAGATCGTGCTGACC |
| | | CAGAGCCCCGGCACCCTGAGCCTGAGCCCCGGCGAGAGGGCCACC |
| | | CTGAGCTGCAGGGCCAGCCAGAGCGTGAGCAGCAGCTACCTGGCC |
| | | TGGTACCAGCAGAAGCCCGGCCAGGCCCCCAGGCTGCTGATCTAC |
| | | GGCGCCAGCAGCAGGGCCACCGGCATCCCCGACAGGTTCAGCGGC |
| | | AGCGGCAGCGGCACCGACTTCACCCTGACCATCAGCAGGCTGGAG |
| | | CCCGAGGACTTCGCCGTGTACTACTGCCAGCAGTACGGCAGCAGCC |
| | | AGTACACCTTCGGCCAGGGCACCAAGCTGGAGATCAAG |
| 76 | 7B1 scFv (VH-linker- | ATGGGCTGGAGCTGCATCATCCTGTTCCTGGTGGCCACCGCCACCG |
| | VL-HIS) with leader | GCGTGCACAGCGAGGTGCACCTGGTGGAGAGTGGTGGCGGTCTGG |
| | sequence | TGCAGCCAGGTGGTAGTCTGAGGCTGAGCTGCGCAGCAAGCGGCT |
| | | TCACCTTCAGCAGGTACTGGATGAGCTGGGTGAGGCAGGCCCAGG |
| | | GCAAGGGCCTGGAGTGGGTGGCCAGCATCAAGCAGGCCGGCAGCG |
| | | AGAAGACCTACGTGGACAGCGTGAAGGGCAGGTTCACCATCAGCAG |
| | | GGACAACGCCAAGAACAGCCTGAGCCTGCAGATGAACAGCCTGAGG |
| | | GCCGAGGACACCGCCGTGTACTACTGCGCCAGGGAGGGCGCCTAC |
| | | TACTACGACAGCGCCAGCTACTACCCCTACTACTACTACTACAGCAT |
| | | GGACGTGTGGGGCCAGGGCACCACCGTGACCGTGAGCAGCGGCG |
| | | GTGGCGGAAGCGGTGGTGGCGGTAGCGGAGGTGGCGGCAGCGAG |
| | | ATCGTGCTGACCCAGAGCCCCGGCACCCTGAGCCTGAGCCCCGGC |
| | | GAGAGGGCCACCCTGAGCTGCAGGGCCAGCCAGAGCGTGAGCAGC |
| | | AGCTACCTGGCCTGGTACCAGCAGAAGCCCGGCCAGGCCCCCAGG |
| | | CTGCTGATCTACGGCGCCAGCAGCAGGGCCACCGGCATCCCCGAC |
| | | AGGTTCAGCGGCAGCGGCAGCGGCACCGACTTCACCCTGACCATCA |
| | | GCAGGCTGGAGCCCGAGGACTTCGCCGTGTACTACTGCCAGCAGTA |
| | | CGGCAGCAGCCAGTACACCTTCGGCCAGGGCACCAAGCTGGAGAT |
| | | CAAGCACCACCACCACCACCACCACCACCACCAC |
| 77 | 7B1 (anti-mesothelin | GAGGTGCACCTGGTGGAGAGTGGTGGCGGTCTGGTGCAGCCAGGT |
| | antibody) | GGTAGTCTGAGGCTGAGCTGCGCAGCAAGCGGCTTCACCTTCAGCA |
| | CAR extracellular | GGTACTGGATGAGCTGGGTGAGGCAGGCCCAGGGCAAGGGCCTGG |
| | domain | AGTGGGTGGCCAGCATCAAGCAGGCCGGCAGCGAGAAGACCTACG |
| | | TGGACAGCGTGAAGGGCAGGTTCACCATCAGCAGGGACAACGCCAA |
| | | GAACAGCCTGAGCCTGCAGATGAACAGCCTGAGGGCCGAGGACAC |
| | | CGCCGTGTACTACTGCGCCAGGGAGGGCGCCTACTACTACGACAGC |
| | | GCCAGCTACTACCCCTACTACTACTACTACAGCATGGACGTGTGGG |
| | | GCCAGGGCACCACCGTGACCGTGAGCAGCGGCGGTGGCGGAAGC |
| | | GGTGGTGGCGGTAGCGGAGGTGGCGGCAGCGAGATCGTGCTGACC |
| | | CAGAGCCCCGGCACCCTGAGCCTGAGCCCCGGCGAGAGGGCCACC |
| | | CTGAGCTGCAGGGCCAGCCAGAGCGTGAGCAGCAGCTACCTGGCC |
| | | TGGTACCAGCAGAAGCCCGGCCAGGCCCCCAGGCTGCTGATCTAC |
| | | GGCGCCAGCAGCAGGGCCACCGGCATCCCCGACAGGTTCAGCGGC |
| | | AGCGGCAGCGGCACCGACTTCACCCTGACCATCAGCAGGCTGGAG |
| | | CCCGAGGACTTCGCCGTGTACTACTGCCAGCAGTACGGCAGCAGCC |
| | | AGTACACCTTCGGCCAGGGCACCAAGCTGGAGATCAAGGGCGGCG |
| | | GCGGCAGCGATTACAAGGACGACGATGACAAGCTGAGCAACTCCAT |
| | | CATGTACTTCAGCCACTTCGTGCCCGTGTTCCTGCCTGCCAAGCCTA |
| | | CAACAACCCCTGCTCCTAGACCTCCTACACCAGCTCCTACAATCGCC |
| | | AGCCAGCCTCTGTCTCTGAGGCCTGAAGCTTGTAGACCTGCTGCTG |
| | | GCGGAGCCGTGCATACCAGAGGACTGGATTTCGCCTGCGAC |
| 78 | 7B1-CD28z | GAGGTGCACCTGGTGGAGAGTGGTGGCGGTCTGGTGCAGCCAGGT |
| | | GGTAGTCTGAGGCTGAGCTGCGCAGCAAGCGGCTTCACCTTCAGCA |
| | | GGTACTGGATGAGCTGGGTGAGGCAGGCCCAGGGCAAGGGCCTGG |
| | | AGTGGGTGGCCAGCATCAAGCAGGCCGGCAGCGAGAAGACCTACG |
| | | TGGACAGCGTGAAGGGCAGGTTCACCATCAGCAGGGACAACGCCAA |
| | | GAACAGCCTGAGCCTGCAGATGAACAGCCTGAGGGCCGAGGACAC |
| | | CGCCGTGTACTACTGCGCCAGGGAGGGCGCCTACTACTACGACAGC |
| | | GCCAGCTACTACCCCTACTACTACTACTACAGCATGGACGTGTGGG |
| | | GCCAGGGCACCACCGTGACCGTGAGCAGCGGCGGTGGCGGAAGC |
| | | GGTGGTGGCGGTAGCGGAGGTGGCGGCAGCGAGATCGTGCTGACC |
| | | CAGAGCCCCGGCACCCTGAGCCTGAGCCCCGGCGAGAGGGCCACC |
| | | CTGAGCTGCAGGGCCAGCCAGAGCGTGAGCAGCAGCTACCTGGCC |
| | | TGGTACCAGCAGAAGCCCGGCCAGGCCCCCAGGCTGCTGATCTAC |
| | | GGCGCCAGCAGCAGGGCCACCGGCATCCCCGACAGGTTCAGCGGC |
| | | AGCGGCAGCGGCACCGACTTCACCCTGACCATCAGCAGGCTGGAG |
| | | CCCGAGGACTTCGCCGTGTACTACTGCCAGCAGTACGGCAGCAGCC |
| | | AGTACACCTTCGGCCAGGGCACCAAGCTGGAGATCAAGGGCGGCG |
| | | GCGGCAGCGATTACAAGGACGACGATGACAAGCTGAGCAACTCCAT |
| | | CATGTACTTCAGCCACTTCGTGCCCGTGTTCCTGCCTGCCAAGCCTA |
| | | CAACAACCCCTGCTCCTAGACCTCCTACACCAGCTCCTACAATCGCC |
| | | AGCCAGCCTCTGTCTCTGAGGCCTGAAGCTTGTAGACCTGCTGCTG |
| | | GCGGAGCCGTGCATACCAGAGGACTGGATTTCGCCTGCGACATCTA |
| | | CATCTGGGCCCCTCTGGCTGGAACATGTGGCGTTTTGCTGCTGAGC |
| | | CTCGTTATCACCCTGTACTGCAACCACCGGAACAGGAGTAAGAGGA |
| | | GCAGGCTCCTGCACAGTGACTACATGAACATGACTCCCCGCCGCCC |
| | | CGGGCCCACCCGCAAGCATTACCAGCCCTATGCCCCACCACGCGAC |
| | | TTCGCAGCCTATCGCTCCCTGAGAGTGAAGTTCAGCAGAAGCGCCG |
| | | ACGCCCCTGCCTATCAGCAGGGCCAGAACCAGCTGTATAACGAGCT |
| | | GAACCTGGGCAGACGGGAAGAGTACGACGTGCTGGACAAGCGGAG |
| | | AGGCAGGGACCCTGAGATGGGCGGCAAGCCCAGAAGAAAGAACCC |
| | | CCAGGAAGGCCTGTATAACGAACTGCAGAAAGACAAGATGGCCGAG |
| | | GCCTACAGCGAGATCGGAATGAAGGGCGAGCGGAGAAGAGGCAAG |
| | | GGCCACGATGGCCTGTACCAGGGCCTGAGCACCGCCACCAAGGAC |
| | | ACCTATGACGCCCTGCACATGCAGGCCCTGCCCCCCAGA |
| 79 | 7B1-41BBz | GAGGTGCACCTGGTGGAGAGTGGTGGCGGTCTGGTGCAGCCAGGT |
| | | GGTAGTCTGAGGCTGAGCTGCGCAGCAAGCGGCTTCACCTTCAGCA |
| | | GGTACTGGATGAGCTGGGTGAGGCAGGCCCAGGGCAAGGGCCTGG |
| | | AGTGGGTGGCCAGCATCAAGCAGGCCGGCAGCGAGAAGACCTACG |
| | | TGGACAGCGTGAAGGGCAGGTTCACCATCAGCAGGGACAACGCCAA |
| | | GAACAGCCTGAGCCTGCAGATGAACAGCCTGAGGGCCGAGGACAC |
| | | CGCCGTGTACTACTGCGCCAGGGAGGGCGCCTACTACTACGACAGC |
| | | GCCAGCTACTACCCCTACTACTACTACTACAGCATGGACGTGTGGG |
| | | GCCAGGGCACCACCGTGACCGTGAGCAGCGGCGGTGGCGGAAGC |
| | | GGTGGTGGCGGTAGCGGAGGTGGCGGCAGCGAGATCGTGCTGACC |
| | | CAGAGCCCCGGCACCCTGAGCCTGAGCCCCGGCGAGAGGGCCACC |
| | | CTGAGCTGCAGGGCCAGCCAGAGCGTGAGCAGCAGCTACCTGGCC |
| | | TGGTACCAGCAGAAGCCCGGCCAGGCCCCCAGGCTGCTGATCTAC |
| | | GGCGCCAGCAGCAGGGCCACCGGCATCCCCGACAGGTTCAGCGGC |
| | | AGCGGCAGCGGCACCGACTTCACCCTGACCATCAGCAGGCTGGAG |
| | | CCCGAGGACTTCGCCGTGTACTACTGCCAGCAGTACGGCAGCAGCC |
| | | AGTACACCTTCGGCCAGGGCACCAAGCTGGAGATCAAGGGCGGCG |
| | | GCGGCAGCGATTACAAGGACGACGATGACAAGCTGAGCAACTCCAT |
| | | CATGTACTTCAGCCACTTCGTGCCCGTGTTCCTGCCTGCCAAGCCTA |
| | | CAACAACCCCTGCTCCTAGACCTCCTACACCAGCTCCTACAATCGCC |
| | | AGCCAGCCTCTGTCTCTGAGGCCTGAAGCTTGTAGACCTGCTGCTG |
| | | GCGGAGCCGTGCATACCAGAGGACTGGATTTCGCCTGCGACATCTA |
| | | CATCTGGGCCCCTCTGGCTGGAACATGTGGCGTTTTGCTGCTGAGC |
| | | CTCGTTATCACCCTGTACTGCAACCACCGGAACAAGCGGGGCAGAA |
| | | AGAAGCTGCTGTATATCTTCAAGCAGCCCTTCATGCGGCCCGTGCA |
| | | GACCACCCAGGAAGAGGACGGCTGCTCCTGCAGATTCCCCGAGGA |
| | | AGAAGAAGGCGGCTGCGAGCTGAGAGTGAAGTTCAGCAGAAGCGC |
| | | CGACGCCCCTGCCTATCAGCAGGGCCAGAACCAGCTGTATAACGAG |
| | | CTGAACCTGGGCAGACGGGAAGAGTACGACGTGCTGGACAAGCGG |
| | | AGAGGCAGGGACCCTGAGATGGGCGGCAAGCCCAGAAGAAAGAAC |
| | | CCCCAGGAAGGCCTGTATAACGAACTGCAGAAAGACAAGATGGCCG |
| | | AGGCCTACAGCGAGATCGGAATGAAGGGCGAGCGGAGAAGAGGCA |
| | | AGGGCCACGATGGCCTGTACCAGGGCCTGAGCACCGCCACCAAGG |
| | | ACACCTATGACGCCCTGCACATGCAGGCCCTGCCCCCCAGA |
| 80 | Linker | GGCGGTGGCGGAAGCGGTGGTGGCGGTAGCGGAGGTGGCGGCAG |
| | | C |
| 81 | Linker variant | GGCGGCGGCGGCAGC |
| 82 | FLAG-tag | GATTACAAGGACGACGATGACAAG |
| 83 | CD8a | CTGAGCAACTCCATCATGTACTTCAGCCACTTCGTGCCCGTGTTCCT |
| | | GCCTGCCAAGCCTACAACAACCCCTGCTCCTAGACCTCCTACACCA |
| | | GCTCCTACAATCGCCAGCCAGCCTCTGTCTCTGAGGCCTGAAGCTT |
| | | GTAGACCTGCTGCTGGCGGAGCCGTGCATACCAGAGGACTGGATTT |
| | | CGCCTGCGACATCTACATCTGGGCCCCTCTGGCTGGAACATGTGGC |
| | | GTTTTGCTGCTGAGCCTCGTTATCACCCTGTACTGCAACCACCGGAA |
| | | C |
| 84 | CD8a hinge | CTGAGCAACTCCATCATGTACTTCAGCCACTTCGTGCCCGTGTTCCT |
| | | GCCTGCCAAGCCTACAACAACCCCTGCTCCTAGACCTCCTACACCA |
| | | GCTCCTACAATCGCCAGCCAGCCTCTGTCTCTGAGGCCTGAAGCTT |
| | | GTAGACCTGCTGCTGGCGGAGCCGTGCATACCAGAGGACTGGATTT |
| | | CGCCTGCGAC |
| 85 | CD8a transmembrane | ATCTACATCTGGGCCCCTCTGGCTGGAACATGTGGCGTTTTGCTGCT |
| | | GAGCCTCGTTATCACCCTGTACTGCAACCACCGGAAC |
| 86 | CD28 | AGGAGTAAGAGGAGCAGGCTCCTGCACAGTGACTACATGAACATGA |
| | | CTCCCCGCCGCCCCGGGCCCACCCGCAAGCATTACCAGCCCTATG |
| | | CCCCACCACGCGACTTCGCAGCCTATCGCTCC |
| 87 | 4-1BB | AAGCGGGGCAGAAAGAAGCTGCTGTATATCTTCAAGCAGCCCTTCAT |
| | | GCGGCCCGTGCAGACCACCCAGGAAGAGGACGGCTGCTCCTGCAG |
| | | ATTCCCCGAGGAAGAAGAAGGCGGCTGCGAGCTG |
| 88 | CD3zeta | CTGAGAGTGAAGTTCAGCAGAAGCGCCGACGCCCCTGCCTATCAGC |
| | | AGGGCCAGAACCAGCTGTATAACGAGCTGAACCTGGGCAGACGGG |
| | | AAGAGTACGACGTGCTGGACAAGCGGAGAGGCAGGGACCCTGAGA |
| | | TGGGCGGCAAGCCCAGAAGAAAGAACCCCCAGGAAGGCCTGTATAA |
| | | CGAACTGCAGAAAGACAAGATGGCCGAGGCCTACAGCGAGATCGGA |
| | | ATGAAGGGCGAGCGGAGAAGAGGCAAGGGCCACGATGGCCTGTAC |
| | | CAGGGCCTGAGCACCGCCACCAAGGACACCTATGACGCCCTGCACA |
| | | TGCAGGCCCTGCCCCCCAGA |
| 89 | CD8a short | ACAACAACCCCTGCTCCTAGACCTCCTACACCAGCTCCTACAATCGC |
| | | CAGCCAGCCTCTGTCTCTGAGGCCTGAAGCTTGTAGACCTGCTGCT |
| | | GGCGGAGCCGTGCATACCAGAGGACTGGATTTCGCCTGCGACATCT |
| | | ACATCTGGGCCCCTCTGGCTGGAACATGTGGCGTTTTGCTGCTGAG |
| | | CCTCGTTATCACCCTGTACTGCAACCACCGGAACATCTACATCTGGG |
| | | CCCCTCTGGCTGGAACATGTGGCGTTTTGCTGCTGAGCCTCGTTATC |
| | | ACCCTGTACTGC |
| 90 | CD8a hinge-variant | ACAACAACCCCTGCTCCTAGACCTCCTACACCAGCTCCTACAATCGC |
| | | CAGCCAGCCTCTGTCTCTGAGGCCTGAAGCTTGTAGACCTGCTGCT |
| | | GGCGGAGCCGTGCATACCAGAGGACTGGATTTCGCCTGCGACATCT |
| | | ACATCTGGGCCCCTCTGGCTGGAACATGTGGCGTTTTGCTGCTGAG |
| | | CCTCGTTATCACCCTGTACTGCAACCACCGGAAC |
| 91 | CD8a-transmembrane | ATCTACATCTGGGCCCCTCTGGCTGGAACATGTGGCGTTTTGCTGCT |
| | variant | GAGCCTCGTTATCACCCTGTACTGC |
| 92 | CD3zeta-variant | AGAGTGAAGTTCAGCAGAAGCGCCGACGCCCCTGCCTATCAGCAGG |
| | | GCCAGAACCAGCTGTATAACGAGCTGAACCTGGGCAGACGGGAAGA |
| | | GTACGACGTGCTGGACAAGCGGAGAGGCAGGGACCCTGAGATGGG |
| | | CGGCAAGCCCAGAAGAAAGAACCCCCAGGAAGGCCTGTATAACGAA |
| | | CTGCAGAAAGACAAGATGGCCGAGGCCTACAGCGAGATCGGAATGA |
| | | AGGGCGAGCGGAGAAGAGGCAAGGGCCACGATGGCCTGTACCAGG |
| | | GCCTGAGCACCGCCACCAAGGACACCTATGACGCCCTGCACATGCA |
| | | GGCCCTGCCCCCCAGA |
| 93 | HFR1 (Chothia, AbM, | GAGGTGCACCTGGTGGAGAGTGGTGGCGGTCTGGTGCAGCCAGGT |
| | IMGT) | GGTAGTCTGAGGCTGAGCTGCGCAGCAAGC |
| 94 | HFR1 (Kabat, Other) | GAGGTGCACCTGGTGGAGAGTGGTGGCGGTCTGGTGCAGCCAGGT |
| | | GGTAGTCTGAGGCTGAGCTGCGCAGCAAGCGGCTTCACCTTCAGC |
| 95 | HFR1 (Contact) | GAGGTGCACCTGGTGGAGAGTGGTGGCGGTCTGGTGCAGCCAGGT |
| | | GGTAGTCTGAGGCTGAGCTGCGCAGCAAGCGGCTTCACCTTC |
| 96 | CDR-H1 (Chothia) | GGCTTCACCTTCAGCAGGTAC |
| 97 | CDR-H1 (AbM) | GGCTTCACCTTCAGCAGGTACTGGATGAGC |
| 98 | CDR-H1 (Kabat, | AGGTACTGGATGAGC |
| | Other) | |
| 99 | CDR-H1 (Contact) | AGCAGGTACTGGATGAGC |
| 100 | CDR-H1 (IMGT) | GGCTTCACCTTCAGCAGGTACTGG |
| 101 | HFR2 (Chothia) | TGGATGAGCTGGGTGAGGCAGGCCCAGGGCAAGGGCCTGGAGTGG |
| | | GTGGCCAGCATC |
| 102 | HFR2 (AbM, Kabat, | TGGGTGAGGCAGGCCCAGGGCAAGGGCCTGGAGTGGGTGGCC |
| | Other) | |
| 103 | HFR2 (Contact) | TGGGTGAGGCAGGCCCAGGGCAAGGGCCTGGAG |
| 104 | HFR2 (IMGT) | ATGAGCTGGGTGAGGCAGGCCCAGGGCAAGGGCCTGGAGTGGGTG |
| | | GCCAGC |
| 105 | CDR-H2 (Chothia) | AAGCAGGCCGGCAGCGAG |
| 106 | CDR-H2 (AbM) | AGCATCAAGCAGGCCGGCAGCGAGAAGACC |
| 107 | CDR-H2 (Kabat, | AGCATCAAGCAGGCCGGCAGCGAGAAGACCTACGTGGACAGCGTG |
| | Other) | AAGGGC |
| 108 | CDR-H2 (Contact) | TGGGTGGCCAGCATCAAGCAGGCCGGCAGCGAGAAGACC |
| 109 | CDR-H2 (IMGT) | ATCAAGCAGGCCGGCAGCGAGAAG |
| 110 | HFR3 (Chothia) | AAGACCTACGTGGACAGCGTGAAGGGCAGGTTCACCATCAGCAGGG |
| | | ACAACGCCAAGAACAGCCTGAGCCTGCAGATGAACAGCCTGAGGGC |
| | | CGAGGACACCGCCGTGTACTACTGCGCCAGG |
| 111 | HFR3 (AbM) | TACGTGGACAGCGTGAAGGGCAGGTTCACCATCAGCAGGGACAACG |
| | | CCAAGAACAGCCTGAGCCTGCAGATGAACAGCCTGAGGGCCGAGG |
| | | ACACCGCCGTGTACTACTGCGCCAGG |
| 112 | HFR3 (Kabat) | AGGTTCACCATCAGCAGGGACAACGCCAAGAACAGCCTGAGCCTGC |
| | | AGATGAACAGCCTGAGGGCCGAGGACACCGCCGTGTACTACTGCG |
| | | CCAGG |
| 113 | HFR3 (Contact) | TACGTGGACAGCGTGAAGGGCAGGTTCACCATCAGCAGGGACAACG |
| | | CCAAGAACAGCCTGAGCCTGCAGATGAACAGCCTGAGGGCCGAGG |
| | | ACACCGCCGTGTACTACTGC |
| 114 | HFR3 (IMGT) | ACCTACGTGGACAGCGTGAAGGGCAGGTTCACCATCAGCAGGGACA |
| | | ACGCCAAGAACAGCCTGAGCCTGCAGATGAACAGCCTGAGGGCCG |
| | | AGGACACCGCCGTGTACTACTGC |
| 115 | HFR3 (Other) | AGGTTCACCATCAGCAGGGACAACGCCAAGAACAGCCTGAGCCTGC |
| | | AGATGAACAGCCTGAGGGCCGAGGAC |
| 116 | CDR-H3 (Chothia, | GAGGGCGCCTACTACTACGACAGCGCCAGCTACTACCCCTACTACT |
| | AbM, Kabat) | ACTACTACAGCATGGACGTG |
| 117 | CDR-H3 (Contact) | GCCAGGGAGGGCGCCTACTACTACGACAGCGCCAGCTACTACCCCT |
| | | ACTACTACTACTACAGCATGGAC |
| 118 | CDR-H3 (IMGT) | GCCAGGGAGGGCGCCTACTACTACGACAGCGCCAGCTACTACCCCT |
| | | ACTACTACTACTACAGCATGGACGTG |
| 119 | CDR-H3 (Other) | ACCGCCGTGTACTACTGCGCCAGGGAGGGCGCCTACTACTACGACA |
| | | GCGCCAGCTACTACCCCTACTACTACTACTACAGCATGGACGTG |
| 120 | HFR4 (Chothia, AbM, | TGGGGCCAGGGCACCACCGTGACCGTGAGCAGC |
| | Kabat, IMGT, Other) | |
| 121 | HFR4 (Contact) | GTGTGGGGCCAGGGCACCACCGTGACCGTGAGCAGC |
| 122 | LFR1 (Chothia, AbM, | GAGATCGTGCTGACCCAGAGCCCCGGCACCCTGAGCCTGAGCCCC |
| | Kabat) | GGCGAGAGGGCCACCCTGAGCTGC |
| 123 | LFR1 (Contact) | GAGATCGTGCTGACCCAGAGCCCCGGCACCCTGAGCCTGAGCCCC |
| | | GGCGAGAGGGCCACCCTGAGCTGCAGGGCCAGCCAGAGCGTG |
| 124 | LFR1 (IMGT) | GAGATCGTGCTGACCCAGAGCCCCGGCACCCTGAGCCTGAGCCCC |
| | | GGCGAGAGGGCCACCCTGAGCTGCAGGGCCAGC |
| 125 | LFR1 (Other) | GAGATCGTGCTGACCCAGAGCCCCGGCACCCTGAGCCTGAGCCCC |
| | | GGCGAGAGGGCCACC |
| 126 | CDR-L1 (Chothia, | AGGGCCAGCCAGAGCGTGAGCAGCAGCTACCTGGCC |
| | AbM, Kabat) | |
| 127 | CDR-L1 (Contact) | AGCAGCAGCTACCTGGCCTGGTAC |
| 128 | CDR-L1 (IMGT) | CAGAGCGTGAGCAGCAGCTAC |
| 129 | CDR-L1 (Other) | CTGAGCTGCAGGGCCAGCCAGAGCGTGAGCAGCAGCTACCTGGCC |
| 130 | LFR2 (Chothia, AbM, | TGGTACCAGCAGAAGCCCGGCCAGGCCCCCAGGCTGCTGATCTAC |
| | Kabat, Other) | |
| 131 | LFR2 (Contact) | CAGCAGAAGCCCGGCCAGGCCCCCAGG |
| 132 | LFR2 (IMGT) | CTGGCCTGGTACCAGCAGAAGCCCGGCCAGGCCCCCAGGCTGCTG |
| | | ATCTAC |
| 133 | CDR-L2 (Chothia, | GGCGCCAGCAGCAGGGCCACC |
| | AbM, Kabat, Other) | |
| 134 | CDR-L2 (Contact) | CTGCTGATCTACGGCGCCAGCAGCAGGGCCACC |
| 135 | CDR-L2 (IMGT) | GGCGCC |
| 136 | LFR3 (Chothia, AbM, | GGCATCCCCGACAGGTTCAGCGGCAGCGGCAGCGGCACCGACTTC |
| | Kabat, Other) | ACCCTGACCATCAGCAGGCTGGAGCCCGAGGACTTCGCCGTGTACT |
| | | ACTGC |
| 137 | LFR3 (Contact) | ACCGGCATCCCCGACAGGTTCAGCGGCAGCGGCAGCGGCACCGAC |
| | | TTCACCCTGACCATCAGCAGGCTGGAGCCCGAGGACTTCGCCGTGT |
| | | ACTACTGC |
| 138 | LFR3 (IMGT) | AGCAGCAGGGCCACCGGCATCCCCGACAGGTTCAGCGGCAGCGGC |
| | | AGCGGCACCGACTTCACCCTGACCATCAGCAGGCTGGAGCCCGAG |
| | | GACTTCGCCGTGTACTACTGC |
| 139 | CDR-L3 (Chothia, | CAGCAGTACGGCAGCAGCCAGTACACC |
| | AbM, Kabat, IMGT, | |
| | Other) | |
| 140 | CDR-L3 (Contact) | CAGCAGTACGGCAGCAGCCAGTAC |
| 141 | LFR4 (Chothia, AbM, | TTCGGCCAGGGCACCAAGCTGGAGATCAAG |
| | Kabat, IMGT, Other) | |
| 142 | LFR4 (Contact) | ACCTTCGGCCAGGGCACCAAGCTGGAGATCAAG |
| 143 | FLAG tag deca- | GDYKDDDDKG |
| | peptide | |
| 144 | FLAG tag | MDYKDDDDK |
| | nonapeptide | |
| 145 | FLAG tag | DFKDDDK |
| | heptapeptide | |
| 146 | FLAG tag alternative | DYKAFDNL |
| | octapeptide | |
| 147 | FLAG tag alternative | DYKDHDG |
| | heptapeptide | |
| 148 | FLAG tag alternative | MDFKDDDDK |
| 149 | FLAG tag alternative | MDYKAFDNL |
| 150 | FLAG tag alternative | DYKDHDI |
| 151 | FLAG tag alternative | DYKDH |
| 152 | FLAG tag alternative | DYKDD |
| 153 | FLAG tag alternative | DYKDHD |
| 154 | FLAG tag alternative | DYKDDD |
| 155 | 7B1-28z-pCDH-EF1a | aacggatctcgacggtatcggttaacttttaaaagaaaaggggggattggggggtacagtgcaggg |
| | plasmid | gaaagaatagtagacataatagcaacagacatacaaactaaagaattacaaaaacaaattacaa |
| | | aattcaaaattttatcgatactagtggatctgcgatcgctccggtgcccgtcagtgggcagagcgc |
| | | acatcgcccacagtccccgagaagttggggggaggggtcggcaattgaacgggtgcctagagaaggt |
| | | ggcgcggggtaaactgggaaagtgatgtcgtgtactggctccgcctttttcccgaggggggggagaa |
| | | ccgtatataagtgcagtagtcgccgtgaacgttctttttcgcaacgggtttgccgccagaacacagc |
| | | tgaagcttcgaggggctcgcatctctccttcacgcgcccgccgccctacctgaggccgccatccacg |
| | | ccggttgagtcgcgttctgccgcctcccgcctgtggtgcctcctgaactgcgtccgccgtctaggta |
| | | agtttaaagctcaggtcgagaccgggcctttgtccggcgctcccttggagcctacctagactcagcc |
| | | ggctctccacgctttgcctgaccctgcttgctcaactctacgtctttgtttcgttttctgttctgcg |
| | | ccgttacagatccaagctgtgaccggcgcctactctagagctagcgaattcTCCACCATGGATTTTC |
| | | AGGTGCAGATTTTCAGCTTCCTGCTAATCAGTGCCTCAGTCATAATGTCTAGAG |
| | | AGGTGCACCTGGTGGAGAGTGGTGGCGGTCTGGTGCAGCCAGGTG |
| | | GTAGTCTGAGGCTGAGCTGCGCAGCAAGCGGCTTCACCTTCAGCAG |
| | | GTACTGGATGAGCTGGGTGAGGCAGGCCCAGGGCAAGGGCCTGGA |
| | | GTGGGTGGCCAGCATCAAGCAGGCCGGCAGCGAGAAGACCTACGT |
| | | GGACAGCGTGAAGGGCAGGTTCACCATCAGCAGGGACAACGCCAA |
| | | GAACAGCCTGAGCCTGCAGATGAACAGCCTGAGGGCCGAGGACAC |
| | | CGCCGTGTACTACTGCGCCAGGGAGGGCGCCTACTACTACGACAGC |
| | | GCCAGCTACTACCCCTACTACTACTACTACAGCATGGACGTGTGGG |
| | | GCCAGGGCACCACCGTGACCGTGAGCAGCGGCGGTGGCGGAAGC |
| | | GGTGGTGGCGGTAGCGGAGGTGGCGGCAGCGAGATCGTGCTGACC |
| | | CAGAGCCCCGGCACCCTGAGCCTGAGCCCCGGCGAGAGGGCCACC |
| | | CTGAGCTGCAGGGCCAGCCAGAGCGTGAGCAGCAGCTACCTGGCC |
| | | TGGTACCAGCAGAAGCCCGGCCAGGCCCCCAGGCTGCTGATCTAC |
| | | GGCGCCAGCAGCAGGGCCACCGGCATCCCCGACAGGTTCAGCGGC |
| | | AGCGGCAGCGGCACCGACTTCACCCTGACCATCAGCAGGCTGGAG |
| | | CCCGAGGACTTCGCCGTGTACTACTGCCAGCAGTACGGCAGCAGCC |
| | | AGTACACCTTCGGCCAGGGCACCAAGCTGGAGATCAAGGGCGGCG |
| | | GCGGCAGCgattacaaggacgacgatgacaagCTGAGCAACTCCATCATGTAC |
| | | TTCAGCCACttcgtgcccgtgttcctgcctgccaagcctacaacaacccctgctcctagacctcct |
| | | acaccagctcctacaatcgccagccagcctctgtctctgaggcctgaagcttgtagacctgctgctgg |
| | | cggagccgtgcataccagaggactggatttcgcctgcgacatctacatctgggcccctctggctggaa |
| | | catgtggcgttttgctgctgagcctcgtgatcaccctgtactgcaaccaccggaacAGGAGTAAG |
| | | AGGAGCAGGCTCCTGCACAGTGACTACATGAACATGACTCCCCGCC |
| | | GCCCCGGGCCCACCCGCAAGCATTACCAGCCCTATGCCCCACCAC |
| | | GCGACTTCGCAGCCTATCGCTCCctgagagtgaagttcagcagaagcgccgacgc |
| | | ccctgcctatcagcagggccagaaccagctgtacaacgagctgaacctgggcagacgggaagag |
| | | tacgacgtgctggacaagcggagaggcagggaccctgagatgggcggcaagcccagaagaaa |
| | | gaacccccaggaaggcctgtataacgaactgcagaaagacaagatggccgaggcctacagcga |
| | | gatcggaatgaagggcgagcggagaagaggcaagggccacgatggcctgtaccagggcctgag |
| | | caccgccaccaaggacacctatgacgccctgcacatgcaggccctgccccccagaggatccgcg |
| | | gcctcgacaatcaacctctggattacaaaatttgtgaaagattgactggtattcttaactatgttgc |
| | | tccttttacgctatgtggatacgctgctttaatgcctttgtatcatgctattgcttcccgtatggct |
| | | ttcattttctcctccttgtataaatcctggttgctgtctctttatgaggagttgtggcccgttgtca |
| | | ggcaacgtggcgtggtgtgcactgtgtttgctgacgcaacccccactggttggggcattgccaccac |
| | | ctgtcagctcctttccgggactttcgctttccccctccctattgccacggcggaactcatcgccgcc |
| | | tgccttgcccgctgctggacaggggctcggctgttgggcactgacaattccgtggtgttgtcgggga |
| | | aatcatcgtcctttccttggctgctcgcctgtgttgccacctggattctgcgcgggacgtccttctg |
| | | ctacgtcccttcggccctcaatccagcggaccttccttcccgcggcctgctgccggctctgcggcct |
| | | cttccgcgtcttcgccttcgccctcagacgagtcggatctccctttgggccgcctccccgcctggta |
| | | cctttaagaccaatgacttacaaggcagctgtagatcttagccactttttaaaagaaaaggggggac |
| | | tggaagggctaattcactcccaacgaagataagatctgctttttgcttgtactgggtctctctggtt |
| | | agaccagatctgagcctgggagctctctggctaactagggaacccactgcttaagcctcaataaagc |
| | | ttgccttgagtgcttcaagtagtgtgtgcccgtctgttgtgtgactctggtaactagagatccctca |
| | | gacccttttagtcagtgtggaaaatctctagcagtagtagttcatgtcatcttattattcagtattt |
| | | ataacttgcaaagaaatgaatatcagagagtgagaggaacttgtttattgcagcttataatggttac |
| | | aaataaagcaatagcatcacaaatttcacaaataaagcatttttttcactgcattctagttgtggtt |
| | | tgtccaaactcatcaatgtatcttatcatgtctggctctagctatcccgcccctaactccgcccatc |
| | | ccgcccctaactccgcccagttccgcccattctccgccccatggctgactaattttttttatttatg |
| | | cagaggccgaggccgcctcggcctctgagctattccagaagtagtgaggaggcttttttggaggcct |
| | | agacttttgcagagaccaaattcgtaatcatgtcatagctgtttcctgtgtgaaattgttatccgc |
| | | tcacaattccacacaacatacgagccggaagcataaagtgtaaagcctggggtgcctaatgagtgag |
| | | ctaactcacattaattgcgttgcgctcactgcccgctttccagtcgggaaacctgtcgtgccagctg |
| | | cattaatgaatcggccaacgcgcggggagaggcggtttgcgtattgggcgctcttccgcttcctcgc |
| | | tcactgactcgctgcgctcggtcgttcggctgcggcgagcggtatcagctcactcaaaggcggtaat |
| | | acggttatccacagaatcaggggataacgcaggaaagaacatgtgagcaaaaggccagcaaaaggcc |
| | | aggaaccgtaaaaaggccgcgttgctggcgtttttccataggctccgcccccctgacgagcatcaca |
| | | aaaatcgacgctcaagtcagaggtggcgaaacccgacaggactataaagataccaggcgtttccccc |
| | | tggaagctccctcgtgcgctctcctgttccgaccctgccgcttaccggatacctgtccgcctttctc |
| | | ccttcgggaagcgtggcgctttctcatagctcacgctgtaggtatctcagttcggtgtaggtcgttc |
| | | gctccaagctgggctgtgtgcacgaaccccccgttcagcccgaccgctgcgccttatccggtaacta |
| | | tcgtcttgagtccaacccggtaagacacgacttatcgccactggcagcagccactggtaacaggatt |
| | | agcagagcgaggtatgtaggcggtgctacagagttcttgaagtggtggcctaactacggctacacta |
| | | gaagaacagtatttggtatctgcgctctgctgaagccagttaccttcggaaaaagagttggtagctc |
| | | ttgatccggcaaacaaaccaccgctggtagcggtggtttttttgtttgcaagcagcagattacgcg |
| | | cagaaaaaaaggatctcaagaagatcctttgatcttttctacggggtctgacgctcagtggaacga |
| | | aaactcacgttaagggattttggtcatgagattatcaaaaaggatcttcacctagatccttttaaa |
| | | ttaaaaatgaagttttaaatcaatctaaagtatatatgagtaaacttggtctgacagttaccaatg |
| | | cttaatcagtgaggcacctatctcagcgatctgtctatttcgttcatccatagttgcctgactccc |
| | | cgtcgtgtagataactacgatacgggagggcttaccatctggccccagtgctgcaatgataccgcga |
| | | gacccacgctcaccggctccagatttatcagcaataaaccagccagccggaagggccgagcgcagaa |
| | | gtggtcctgcaactttatccgcctccatccagtctattaattgttgccgggaagctagagtaagtag |
| | | ttcgccagttaatagtttgcgcaacgttgttgccattgctacaggcatcgtggtgtcacgctcgtcg |
| | | tttggtatggcttcattcagctccggttcccaacgatcaaggcgagttacatgatcccccatgttgt |
| | | gcaaaaaagcggttagctccttcggtcctccgatcgttgtcagaagtaagttggccgcagtgttatc |
| | | actcatggttatggcagcactgcataattctcttactgtcatgccatccgtaagatgcttttctgtg |
| | | actggtgagtactcaaccaagtcattctgagaatagtgtatgcggcgaccgagttgctcttgcccgg |
| | | cgtcaatacgggataataccgcgccacatagcagaactttaaaagtgctcatcattggaaaacgttc |
| | | ttcggggcgaaaactctcaaggatcttaccgctgttgagatccagttcgatgtaacccactcgtgca |
| | | cccaactgatcttcagcatcttttactttcaccagcgtttctgggtgagcaaaaacaggaaggcaaa |
| | | aatgccgcaaaaagggaataagggcgacacggaaatgttgaatactcatactcttcctttttcaata |
| | | ttattgaagcatttatcagggttattgtctcatgagcggatacatatttgaatgtatttagaaaaat |
| | | aaacaaataggggttccgcgcacatttccccgaaaagtgccacctgacgtctaagaaaccattatta |
| | | tcatgacattaacctataaaaataggcgtatcacgaggccctttcgtctcgcgcgtttcggtgatga |
| | | cggtgaaaacctctgacacatgcagctcccggagacggtcacagcttgtctgtaagcggatgccgg |
| | | gagcagacaagcccgtcagggcgcgtcagcgggtgttggcgggtgtcggggctggcttaactatgcg |
| | | gcatcagagcagattgtactgagagtgcaccatatgcggtgtgaaataccgcacagatgcgtaagg |
| | | agaaaataccgcatcaggcgccattcgccattcaggctgcgcaactgttgggaagggcgatcggtgc |
| | | gggcctcttcgctattacgccagctggcgaaagggggatgtgctgcaaggcgattaagttgggtaac |
| | | gccagggttttcccagtcacgacgttgtaaaacgacggccagtgccaagctgacgcgtgtagtctta |
| | | tgcaatactcttgtagtcttgcaacatggtaacgatgagttagcaacatgccttacaaggagagaaa |
| | | aagcaccgtgcatgccgattggtggaagtaaggtggtacgatcgtgccttattaggaaggcaacag |
| | | acgggtctgacatggattggacgaaccactgaattgccgcattgcagagatattgtatttaagtgcc |
| | | tagctcgatacataaacgggtctctctggttagaccagatctgagcctgggagctctctggctaact |
| | | agggaacccactgcttaagcctcaataaagcttgccttgagtgcttcaagtagtgtgtgcccgtctg |
| | | ttgtgtgactctggtaactagagatccctcagacccttttagtcagtgtggaaaatctctagcagtg |
| | | gcgcccgaacagggacttgaaagcgaaagggaaaccagaggagctctctcgacgcaggactcggctt |
| | | gctgaagcgcgcacggcaagaggcgaggggcggcgactggtgagtacgccaaaaattttgactagcg |
| | | gaggctagaaggagagagatgggtgcgagagcgtcagtattaagcgggggagaattagatcgcgatg |
| | | ggaaaaaattcggttaaggccagggggaaagaaaaaatataaattaaaacatatagtatgggcaagc |
| | | agggagctagaacgattcgcagttaatcctggcctgttagaaacatcagaaggctgtagacaaatac |
| | | tgggacagctacaaccatcccttcagacaggatcagaagaacttagatcattatataatacagtagc |
| | | aaccctctattgtgtgcatcaaaggatagagataaaagacaccaaggaagctttagacaagatagag |
| | | ggaagagcaaaacaaaataagaccaccgcacagcaagcggccactgatcttcagacctggaggagga |
| | | gatatgagggacaattggagaagtgaattatataaatataaagtagtaaaaattgaaccattaggag |
| | | tagcacccaccaaggcaaagagaagagtggtgcagagagaaaaaagagcagtgggaataggagcttt |
| | | gttccttgggttcttgggagcagcaggaagcactatgggcgcagcgtcaatgacgctgacggtacag |
| | | gccagacaattattgtctggtatagtgcagcagcagaacaatttgctgagggctattgaggcgcaac |
| | | agcatctgttgcaactcacagtctggggcatcaagcagctccaggcaagaatcctggctgtggaaa |
| | | gatacctaaaggatcaacagctcctggggatttggggttgctctggaaaactcatttgcaccactgc |
| | | tgtgccttggaatgctagttggagtaataaatctctggaacagatttggaatcacacgacctggatg |
| | | gagtgggacagagaaattaacaattacacaagcttaatacactccttaattgaagaatcgcaaaacc |
| | | agcaagaaaagaatgaacaagaattattggaattagataaatgggcaagtttgtggaattggtttaa |
| | | cataacaaattggctgtggtatataaaattattcataatgatagtaggaggcttggtaggtttaaga |
| | | atagtttttgctgtactttctatagtgaatagagttaggcagggatattcaccattatcgtttcag |
| | | acccacctcccaaccccgaggggacccgacaggcccgaaggaatagaagaagaaggtggagagaga |
| | | gacagagacagatccattcgattagtg |
| 156 | 7B1-28z codon | ATGGATTTTCAAGTCCAAATATTTAGTTTTCTCTTGATTAGCGCAAGC |
| | optimised | GTGATAATGAGCCGCGAGGTCCATCTTGTCGAGTCCGGAGGTGGTT |
| | | TGGTGCAACCTGGCGGCTCCTTGCGGCTCTCCTGTGCTGCGTCTGG |
| | | CTTCACGTTTTCTAGGTACTGGATGAGCTGGGTACGCCAGGCCCAA |
| | | GGAAAGGGGCTTGAGTGGGTAGCTAGTATAAAGCAGGGGGGATCC |
| | | GAGAAAACTTACGTCGACTCTGTCAAAGGACGCTTTACCATATCCCG |
| | | CGATAACGCAAAAAATTCACTGAGTCTGCAGATGAACTCTCTGAGAG |
| | | CAGAAGATACAGCTGTGTATTATTGCGCGCGAGAAGGAGCATATTAT |
| | | TATGATTCCGCATCATACTATCCATACTACTATTACTACTCAATGGAT |
| | | GTCTGGGGCCAGGGCACAACCGTTACGGTTAGCAGCGGTGGGGGT |
| | | GGCTCCGGGGGCGGGGGCTCAGGAGGCGGTGGCAGCGAAATTGTA |
| | | TTGACGCAGAGTCCAGGCACTTTGTCTCTGTCTCCAGGGGAAAGGG |
| | | CTACGTTGTCATGCCGAGCTTCTCAGTCAGTAAGTAGTTCCTACCTC |
| | | GCCTGGTATCAGCAGAAACCGGGCCAAGCACCAAGACTTCTGATTT |
| | | ATGGGGCGTCTTCCAGGGCCACCGGTATCCCTGACAGATTTTCCGG |
| | | GTCCGGAAGTGGAACTGATTTTACCCTTACCATTAGTCGCCTCGAGC |
| | | CAGAAGACTTTGCCGTGTATTATTGCCAGCAGTACGGTAGTTCCCAA |
| | | TACACATTTGGCCAAGGCACGAAACTGGAAATAAAAGGCGGTGGCG |
| | | GATCAGATTATAAGGATGATGACGATAAATTGTCAAACTCTATAATGT |
| | | ACTTCTCCCACTTTGTACCCGTATTTCTGCCTGCAAAACCCACCACTA |
| | | CCCCGGCGCCTCGACCTCCCACGCCAGCTCCTACTATAGCTTCACA |
| | | GCCTCTCTCACTCCGACCAGAAGCTTGTAGACCGGCCGCAGGCGGA |
| | | GCTGTCCACACTAGGGGGCTCGACTTTGCTTGTGATATCTACATTTG |
| | | GGCTCCACTTGCCGGCACTTGTGGGGTCCTCCTCTTGAGTCTCGTA |
| | | ATCACACTTTATTGCAATCATCGGAATAGATCCAAACGATCCAGACT |
| | | GCTGCACTCCGATTATATGAACATGACGCCACGGAGACCAGGACCA |
| | | ACAAGGAAACATTACCAGCCATACGCCCCACCCCGAGACTTTGCAG |
| | | CCTACAGGTCCCTCCGGGTTAAATTCAGCAGGTCTGCCGATGCTCC |
| | | AGCATACCAGCAAGGGCAGAATCAACTTTACAACGAGCTTAATCTTG |
| | | GCCGGAGGGAAGAATATGATGTGCTGGATAAGCGGAGGGGCCGAG |
| | | ACCCTGAAATGGGAGGGAAACCGCGACGCAAAAACCCTCAAGAGGG |
| | | GCTTTATAACGAGCTGCAAAAGGACAAAATGGCTGAGGCTTACAGTG |
| | | AGATAGGCATGAAAGGAGAGAGAAGGCGGGGGAAGGGGCACGACG |
| | | GCCTGTATCAGGGGCTTAGCACGGCAACTAAGGACACCTACGACGC |
| | | TTTGCATATGCAGGCACTCCCGCCTAGA |

### DETAILED DESCRIPTION OF THE EMBODIMENTS

It will be understood that the invention disclosed and defined in this specification extends to all alternative combinations of two or more of the individual features mentioned or evident from the text or drawings. All of these different combinations constitute various alternative aspects of the invention.

Further aspects of the present invention and further embodiments of the aspects described in the preceding paragraphs will become apparent from the following description, given by way of example and with reference to the accompanying drawings.

Reference will now be made in detail to certain embodiments of the invention. While the invention will be described in conjunction with the embodiments, it will be understood that the intention is not to limit the invention to those embodiments. On the contrary, the invention is intended to cover all alternatives, modifications, and equivalents, which may be included within the scope of the present invention as defined by the claims.

### General

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or groups of compositions of matter. Thus, as used herein, the singular forms “a”, “an” and “the” include plural aspects, and vice versa, unless the context clearly dictates otherwise. For example, reference to “a” includes a single as well as two or more; reference to “an” includes a single as well as two or more; reference to “the” includes a single as well as two or more and so forth.

Those skilled in the art will appreciate that the present invention is susceptible to variations and modifications other than those specifically described. It is to be understood that the invention includes all such variations and modifications. The invention also includes all of the steps, features, compositions and compounds referred to or indicated in this specification, individually or collectively, and any and all combinations or any two or more of said steps or features.

One skilled in the art will recognize many methods and materials similar or equivalent to those described herein, which could be used in the practice of the present invention. The present invention is in no way limited to the methods and materials described.

All of the patents and publications referred to herein are incorporated by reference in their entirety.

The present invention is not to be limited in scope by the specific examples described herein, which are intended for the purpose of exemplification only. Functionally-equivalent products, compositions and methods are clearly within the scope of the present invention.

Any example or embodiment of the present invention herein shall be taken to apply mutatis mutandis to any other example or embodiment of the invention unless specifically stated otherwise.

Unless specifically defined otherwise, all technical and scientific terms used herein shall be taken to have the same meaning as commonly understood by one of ordinary skill in the art (for example, in cell culture, molecular genetics, immunology, immunohistochemistry, protein chemistry, and biochemistry).

Unless otherwise indicated, the recombinant protein, cell culture, and immunological techniques utilized in the present disclosure are standard procedures, well known to those skilled in the art. Such techniques are described and explained throughout the literature in sources such as, J. Perbal, A Practical Guide to Molecular Cloning, John Wiley and Sons (1984), J. Sambrook et al. Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

The term “and/or”, e.g., “X and/or Y” shall be understood to mean either “X and Y” or “X or Y” and shall be taken to provide explicit support for both meanings or for either meaning.

As used herein the term “derived from” shall be taken to indicate that a specified integer may be obtained from a particular source albeit not necessarily directly from that source.

When comparing amino acid sequences, the sequences should be compared over a comparison window which is determined by the length of the polypeptide. The comparison window may comprise additions or deletions (i.e. gaps) of about 20% or less as compared to the reference sequence (which does not comprise additions or deletions) for optimal alignment of the two sequences. Optimal alignment of sequences for aligning a comparison window may be conducted by computerised implementations of algorithms such as the BLAST family of programs as, for example, disclosed by Altschul et al., 1997, Nucl. Acids Res. 25: 3389-3402. Global alignment programs may also be used to align similar sequences of roughly equal size. Examples of global alignment programs include NEEDLE (available at www.ebi.ac.uk/Tools/psa/emboss_needle/) which is part of the EMBOSS package (Rice P et al., 2000, Trends Genet., 16: 276-277), and the GGSEARCH program (available at fasta.bioch.virginia.edu/fasta_www2/fasta_www.cgi?rm=compare&pgm=gnw) which is part of the FASTA package (Pearson W and Lipman D, 1988, Proc. Natl. Acad. Sci. USA, 85: 2444-2448). Both of these programs are based on the Needleman-Wunsch algorithm which is used to find the optimum alignment (including gaps) of two sequences along their entire length. A detailed discussion of sequence analysis can also be found in Unit 19.3 of Ausubel et al (“Current Protocols in Molecular Biology” John Wiley & Sons Inc, 1994-1998, Chapter 15, 1998).

“Antibodies” or “immunoglobulins” or “Igs” are gamma globulin proteins that are found in blood, or other bodily fluids of vertebrates that function in the immune system to bind antigen, hence identifying and/or neutralising foreign objects.

Antibodies are generally a heterotetrameric glycoprotein composed of two identical light (L) chains and two identical heavy (H) chains. Each L chain is linked to a H chain by one covalent disulfide bond. The two H chains are linked to each other by one or more disulfide bonds depending on the H chain isotype. Each H and L chain also has regularly spaced intrachain disulfide bridges.

H and L chains define specific Ig domains. More particularly, each H chain has at the N-terminus, a variable domain (V_{H}) followed by three constant domains (CH) for each of the α and γ chains and four CH domains for μ and ε isotypes. Each L chain has at the N-terminus, a variable domain (V_{L}) followed by a constant domain (CL) at its other end. The V_{L} is aligned with the V_{H} and the C_{L} is aligned with the first constant domain of the heavy chain (CH1).

Antibodies can be assigned to different classes or isotypes. There are five classes of immunoglobulins: IgA, IgD, IgE, IgG, and IgM, having heavy chains designated α, δ, ε, γ, and μ, respectively. The γ and α classes are further divided into subclasses on the basis of relatively minor differences in ¾ sequence and function, e.g., humans express the following subclasses: IgG1, IgG2, IgG3, IgG4, IgAI, and IgA2. The L chain from any vertebrate species can be assigned to one of two clearly distinct types, called kappa and lambda, based on the amino acid sequences of their constant domains.

The constant domain includes the Fc portion that comprises the carboxy-terminal portions of both H chains held together by disulfides. The effector functions of antibodies such as ADCC are determined by sequences in the Fc region, which region is also the part recognised by Fc receptors (FcR) found on certain types of cells.

The pairing of a V_{H} and V_{L} together forms a “variable region” or “variable domain” including the amino-terminal domains of the heavy or light chain of the antibody. The variable domain of the heavy chain may be referred to as “V_{H}.” The variable domain of the light chain may be referred to as “V_{L}.” The V domain contains an “antigen binding site” that affects antigen binding and defines specificity of a particular antibody for its particular antigen. V regions span about 110 amino acid residues and consist of relatively invariant stretches called framework regions (FRs) (generally about 4) of 15-30 amino acids separated by shorter regions of extreme variability called “hypervariable regions” (generally about 3) that are each generally 9-12 amino acids long. The FRs largely adopt a β-sheet configuration and the hypervariable regions form loops connecting, and in some cases forming part of, the β-sheet structure.

“Hypervariable region” refers to the regions of an antibody variable domain that are hypervariable in sequence and/or form structurally defined loops. Generally, antibodies comprise six hypervariable regions; three in the V_{H} (H1, H2, H3), and three in the V_{L} (L1, L2, L3).

“Framework” or “FR” residues are those variable domain residues other than the hypervariable region residues herein defined.

An “antigen binding site” generally refers to a molecule that includes at least the hypervariable and framework regions that are required for imparting antigen binding function to a V domain. An antigen binding site may be in the form of an antibody or an antibody fragment, (such as a mAb, single domain (SD)-mAb, dAb, Fab, SD-Fab, Fd, SD-Fv, Fv, F(ab′)2 or scFv) in a method described herein.

An “intact” or “whole” antibody is one that comprises an antigen-binding site as well as a C_{L} and at least heavy chain constant domains, CH1, CH2 and CH3. The constant domains may be native sequence constant domains (e.g. human native sequence constant domains) or amino acid sequence variant thereof.

“Whole antibody fragments including a variable domain” include SD-mAb, Fab, Fab′, F(ab′)2, and Fv fragments; diabodies; linear antibodies, single-chain antibody molecules; and multi-specific antibodies formed from antibody fragments.

The “Fab fragment” consists of an entire L chain along with the variable region domain of the H chain (V_{H}), and the first constant domain of one heavy chain (CH1). Each Fab fragment is monovalent with respect to antigen binding, i.e., it has a single antigen-binding site.

A “Fab′ fragment” differs from Fab fragments by having additional few residues at the carboxy terminus of the CH1 domain including one or more cysteines from the antibody hinge region. Fab′-SH is the designation herein for Fab′ in which the cysteine residue(s) of the constant domains bear a free thiol group.

A “F(ab′)2 fragment” roughly corresponds to two disulphide linked Fab fragments having divalent antigen-binding activity and is still capable of cross-linking antigen.

An “Fv” is the minimum antibody fragment that contains a complete antigen-recognition and binding site. This fragment consists of a dimer of one heavy and one light chain variable region domain in tight, non-covalent association.

In a single-chain Fv (scFv) species, one heavy and one light chain variable domain can be covalently linked by a flexible peptide linker such that the light and heavy chains can associate in a “dimeric” structure analogous to that in a two-chain Fv species. From the folding of these two domains emanate six hypervariable loops (3 loops each from the H and L chain) that contribute the amino acid residues for antigen binding and confer antigen binding specificity to the antibody.

“Single-chain Fv” also abbreviated as “sFv” or “scFv” are antibody fragments that comprise the V_{H} and V_{L} antibody domains connected to form a single polypeptide chain. Preferably, the scFv polypeptide further comprises a polypeptide linker between the V_{H} and V_{L} domains that enables the scFv to form the desired structure for antigen binding.

A “single variable domain” is half of an Fv (comprising only three CDRs specific for an antigen) that has the ability to recognise and bind antigen, although generally at a lower affinity than the entire binding site.

“Diabodies” refers to antibody fragments with two antigen-binding sites, which fragments comprise a heavy-chain variable domain (V_{H}) connected to a light-chain variable domain (V_{L}) in the same polypeptide chain (V_{H}-V_{L}). The small antibody fragments are prepared by constructing sFv fragments (see preceding paragraph) with short linkers (about 5-10 residues) between the V_{H} and V_{L} domains such that interchain but not intra-chain pairing of the V domains is achieved, resulting in a bivalent fragment, i.e., a fragment having two antigen-binding sites.

Diabodies may be bivalent or bispecific. Bispecific diabodies are heterodimers of two “crossover” sFv fragments in which the V_{H} and V_{L} domains of the two antibodies are present on different polypeptide chains. Triabodies and tetrabodies are also generally known in the art.

An “isolated antibody” is one that has been identified and separated and/or recovered from a component of its pre-existing environment. Contaminant components are materials that would interfere with therapeutic uses for the antibody, and may include enzymes, hormones, and other proteinaceous or non-proteinaceous solutes.

A “human antibody” refers to an antibody that possesses an amino acid sequence that corresponds to that of an antibody produced by a human. Human antibodies can be produced using various techniques known in the art, including phage—display libraries. Human antibodies can be prepared by administering the antigen to a transgenic animal that has been modified to produce such antibodies in response to antigenic challenge, but whose endogenous loci have been disabled.

“Humanised’ forms of non-human (e.g., rodent) antibodies are chimeric antibodies that contain minimal sequence derived from the non-human antibody. For the most part, humanised antibodies are human immunoglobulins (recipient antibody) in which residues from a hypervariable region of the recipient are replaced by residues from a hypervariable region of a non-human species (donor antibody) such as mouse, rat, rabbit or non-human primate having the desired antibody specificity, affinity, and capability. In some instances, framework region (FR) residues of the human immunoglobulin are replaced by corresponding non-human residues. Furthermore, humanised antibodies may comprise residues that are not found in the recipient antibody or in the donor antibody. These modifications are made to further refine antibody performance. In general, the humanised antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanised antibody optionally also will comprise at least a portion of an immunoglobulin constant region (Fc), typically that of a human immunoglobulin.

“Monoclonal antibody” refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site or determinant on the antigen. In addition to their specificity, the monoclonal antibodies are advantageous in that they may be synthesised uncontaminated by other antibodies. Monoclonal antibodies may be prepared by the hybridoma methodology. The “monoclonal antibodies” may also be isolated from phage antibody libraries using molecular engineering techniques.

“Binding affinity” generally refers to the strength of the sum total of non-covalent interactions between a single binding site of a molecule (e.g., an antibody) and its binding partner (e.g., an antigen). Unless indicated otherwise, as used herein, “binding affinity” refers to intrinsic binding affinity, which reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present invention.

As used herein, the term “antigen” is intended to include substances that bind to or evoke the production of one or more antibodies and may comprise, but is not limited to, proteins, peptides, polypeptides, oligopeptides, lipids, carbohydrates, and combinations thereof, for example a glycosylated protein or a glycolipid. The term “antigen” as used herein refers to a molecular entity that may be expressed on a target cell and that can be recognised by means of the adaptive immune system including but not restricted to antibodies or TCRs, or engineered molecules including but not restricted to transgenic TCRs, CARs, scFvs or multimers thereof, Fab-fragments or multimers thereof, antibodies or multimers thereof, single chain antibodies or multimers thereof, or any other molecule that can execute binding to a structure with high affinity.

“Epitope” generally refers to that part of an antigen that is bound by the antigen binding site of an antibody. An epitope may be “linear” in the sense that the hypervariable loops of the antibody CDRs that form the antigen binding site bind to a sequence of amino acids as in a primary protein structure. In certain embodiments, the epitope is a “conformational epitope” i.e. one in which the hypervariable loops of the CDRs bind to residues as they are presented in the tertiary or quaternary protein structure.

The term “target cell” as used herein refers to a cell that expresses mesothelin. The target cell may be a cancer cell or any other diseased cell.

The term “disorder” or “condition” means a functional abnormality or disturbance in a subject such as a cancer, an autoimmune disorder, or an infection by virus, bacteria, parasite, or others.

For example, a nucleic acid or a peptide naturally present in a living animal is not “isolated”, but the same nucleic acid or peptide partially or completely separated from the coexisting materials of its natural state is “isolated”. An isolated nucleic acid or protein can also exist in a non-native environment such as, for example, in a host cell.

The term “autologous” as used herein refers to any material derived from the same subject to whom it is later re-introduced.

The term “allogeneic” as used herein refers to any material derived from a different subject of the same species as the subject to whom the material is re-introduced.

The terms “therapeutically effective amount” or “therapeutically effective population” mean an amount of, for example, a cell population that provides a therapeutic benefit in a subject.

The terms “binds to”, “specifically binds to” or “specific for” with respect to a CAR refers to an antigen-binding domain that recognises and binds to a specific antigen, does not substantially recognise or bind to other molecules in a sample. An antigen-binding domain that binds specifically to an antigen from one species also may bind to that antigen from another species. This cross-species reactivity is typical of many antibodies and therefore not contrary to the definition that the antigen-binding domain is specific. An antigen-binding domain that specifically binds to an antigen may bind also to different allelic forms of the antigen (allelic variants, splice variants, isoforms etc.) or homologous variants of this antigen from the same gene family. This cross reactivity is typical of many antibodies and therefore not contrary to the definition that the antigen-binding domain is specific.

The terms “engineered cell” and “genetically modified cell” as used herein can be used interchangeably. The terms mean containing and/or expressing a foreign gene or nucleic acid sequence that in turn modifies the genotype or phenotype of the cell or its progeny. Especially, the terms refer to the fact that cells, preferentially immune cells, can be manipulated by recombinant methods well known in the art to express stably or transiently peptides or proteins that are not expressed in these cells in the natural state. For example, immune cells are engineered to express an artificial construct such as a chimeric antigen receptor on their cell surface. For example, the CAR sequences may be delivered into cells using an adenoviral, adeno-associated viral (AAV)-based, retroviral or lentiviral vector or any other pseudotyped variations thereof or any other gene delivery mechanism such as electroporation or lipofection with CRISPR/Cas9, transposons (e.g. sleeping-beauty) or variations thereof. The gene delivery may be in the form of mRNA (transient) or DNA (transient or permanent).

The terms “immune cell” or “immune effector cell” refer to a cell that may be part of the immune system and executes a particular effector function such as alpha-beta T cells, NK cells, NKT cells, B cells, Breg cells, Treg cells, innate lymphoid cells (ILC), cytokine induced killer (CIK) cells, lymphokine activated killer (LAK) cells, gamma-delta T cells, mesenchymal stem cells or mesenchymal stromal cells (MSC), monocytes or macrophages or any hematopoietic progenitor cells such as pluripotent stem cells and early progenitor subsets that may mature or differentiate into somatic cells. The cells may be naturally occurring or generated by cytokine exposure, artificial/genetically modified cells (such as iPSCs and other artificial cell types). The immune cell may be an artificial cell subset including induced pluripotent stem cells and cells maturated therefrom. Preferred immune cells are cells with cytotoxic effector function such as alpha-beta T cells, NK cells, NKT cells, ILC, CIK cells, LAK cells or gamma-delta T cells. “Effector function” means a specialised function of a cell, e.g. in a T cell an effector function may be cytolytic activity or helper cell activity including the secretion of cytokines.

### Chimeric Antigen Receptor (CAR)

Generally, an “antigen binding domain” refers to the region of the CAR that specifically binds to an antigen (and thereby is able to target a cell containing the antigen). The CARs of the invention may comprise one or more antigen binding domains. Generally, the targeting regions on the CAR are extracellular. The antigen binding domain may comprise an antibody or an antibody binding fragment thereof. The antigen binding domain may comprise, for example, full length heavy chain, Fab fragments, single chain Fv (scFv) fragments, divalent single chain antibodies or diabodies. Any molecule that binds specifically to a given antigen such as affibodies or ligand binding domains from naturally occurring receptors may be used as an antigen binding domain. Often the antigen binding domain is a scFv. Normally, in a scFv the variable regions of an immunoglobulin heavy chain and light chain are fused by a flexible linker to form a scFv. Such a linker may be for example the “(G₄/S₁)₃-linker” and variations thereof but the skilled person will appreciate that various linker sequences and formats may be used.

In some instances, it is beneficial for the antigen binding domain to be derived from the same species in which the CAR will be used in. For example, when it is planned to use it therapeutically in humans, it may be beneficial for the antigen binding domain of the CAR to comprise a human or humanised antibody or antigen binding fragment thereof. Human or humanised antibodies or antigen binding fragments thereof can be made by a variety of methods well known in the art.

A “signal peptide” refers to a peptide sequence that directs the transport and localisation of the protein within a cell, e.g. to a certain cell organelle (such as the endoplasmic reticulum) and/or the cell surface.

“Spacer” or “hinge” as used herein refers to the hydrophilic region that is between the antigen binding domain and the transmembrane domain. The CARs of the invention may comprise an extracellular spacer domain but it is also possible to leave out such a spacer. The spacer may include e.g. Fc fragments of antibodies or fragments thereof, hinge regions of antibodies or fragments thereof, CH2 or CH3 regions of antibodies, accessory proteins, artificial spacer sequences or combinations thereof. A prominent example of a spacer is the CD8a (CD8alpha) hinge.

The transmembrane domain of the CAR may be derived from any desired natural or synthetic source for such a domain. When the source is natural, the domain may be derived from any membrane-bound or transmembrane protein. The transmembrane domain may be derived for example from CD8alpha or CD28.

The cytoplasmic domain (or the intracellular signaling domain) of the CAR is responsible for activation of at least one of the normal effector functions of the immune cell in which the CAR is expressed. “Effector function” means a specialised function of a cell, e.g. in a T cell an effector function may be cytolytic activity or helper cell activity including the secretion of cytokines. The intracellular signalling domain refers to the part of a protein that transduces the effector function signal and directs the cell expressing the CAR to perform a specialised function. The intracellular signalling domain may include any complete, mutated or truncated part of the intracellular signalling domain of a given protein sufficient to transduce a signal that initiates or blocks immune cell effector functions.

Linkers

A linker may be a peptide having a length of up to 20 amino acids. The term “linked to” or “fused to” refers to a covalent bond, e.g., a peptide bond, formed between two moieties. Accordingly, in the context of the present invention the linker may have a length of 1, 2, 3, 4, 5, 6, 7, 8, 9, 10, 11, 12, 13, 14, 15, 16, 17, 18, 19, 20, 21 or 22 amino acids. For example, the herein provided CAR may comprise a linker between the VH and VL of the antigen binding domain and/or the antigen binding domain and the hinge. Such linkers have the advantage that they can make it more likely that the different polypeptides of the fusion protein fold independently and behave as expected.

The skilled person will be familiar with the design and use of various peptide linkers comprised of various amino acids, and of various lengths, which would be suitable for use as linkers in accordance with the present invention. The linker may comprise various combinations of repeated amino acid sequences. The linker may be a flexible linker (such as those comprising repeats of glycine and serine residues), a rigid linker (such as those comprising glutamic acid and lysine residues, flanking alanine repeats) and/or a cleavable linker (such as sequences that are susceptible by protease cleavage).

The peptide linker may be any one or more repeats of Gly-Ser (GS), Gly-Gly-Ser (GGS), Gly-Gly-Gly-Ser (GGGS) or Gly-Gly-Gly-Gly-Ser (GGGGS) or variations thereof. In any embodiment, the linker may comprise or consist of the sequence GGGGSGGGGSGGGGS, i.e. (G4S)₃.

In any embodiment, the peptide linker can include the amino acid sequence GGGGGS (a linker of 6 amino acids in length) or even longer. The linker may be a series of repeating glycine and serine residues (GS) of different lengths, i.e., (GS)n where n is any number from 1 to 15 or more. For example, the linker may be (GS)₃ (i.e., GSGSGS) or longer (GS)₁₁ or longer. It will be appreciated that n can be any number including 1, 2, 3, 4, 5, 6, 7, 8, 9, 10, 11 or more.

Epitope Tags

Epitope or affinity tags are short stretches of amino acids to which a specific antibody can be raised, which in some embodiments allows one to specifically identify and track the tagged protein that has been added to a living organism or to cultured cells. Detection of the tagged molecule can be achieved using a number of different techniques. Examples of such techniques include: immunohistochemistry, immunoprecipitation, flow cytometry, immunofluorescence microscopy, ELISA, immunoblotting (“western”), and affinity chromatography. Epitope tags add a known epitope (e.g. antibody binding site) on the subject protein, to provide binding of a known and often high-affinity antibody, and thereby allowing one to specifically identify and track the tagged protein that has been added to a living organism or to cultured cells.

Examples of epitope tags include the myc, T7, GST, GFP, HA (hemagglutinin) and FLAG tags. The first four examples are epitopes derived from existing molecules. In contrast, FLAG is a synthetic epitope tag designed for high antigenicity (see, e.g., U.S. Pat. Nos. 4,703,004 and 4,851,341). The myc tag was used in some particular examples disclosed herein because high quality reagents are available to be used for its detection. Epitope tags can of course have one or more additional functions, beyond recognition by an antibody.

Further illustrative examples of such epitopes or affinity tags include, peptide tags (e.g., FLAG-tag, HA-tag, His-tag, Myc-tag, S-tag, SBP-tag, Strep-tag, eXact-tag) and protein tags (e.g., GST-tag, MBP-tag, GFP-tag). In an embodiment, the epitope or affinity tag is a His-tag.

### Nucleic Acids

In another aspect, the present invention provides a nucleic acid molecule including a nucleotide sequence encoding the chimeric antigen receptor according to the invention. In some embodiments, the nucleic acid molecule is a non-naturally occurring nucleic acid molecule.

In some embodiments of the invention, the nucleic acid molecule includes a nucleotide sequence which encodes the amino acid sequence set forth in SEQ ID NO: 6, SEQ ID NO: 7 or SEQ ID NO: 8, or encodes a functional variant of SEQ ID NO: 6, SEQ ID NO: 7 or SEQ ID NO: 8. In some embodiments, the functional variant includes an amino acid sequence which is at least 80% identical to SEQ ID NO: 6, SEQ ID NO: 7 or SEQ ID NO: 8.

The nucleic acid molecule may comprise any polyribonucleotide or polydeoxyribonucleotide, which may be unmodified, or modified, RNA or DNA. For example, the nucleic acid molecule may include single- and/or double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, the nucleic acid molecule may comprise triple-stranded regions comprising RNA or DNA or both RNA and DNA. The nucleic acid molecule may also comprise one or more modified bases or DNA or RNA backbones modified for stability or for other reasons. A variety of modifications can be made to DNA and RNA; thus the term “nucleic acid molecule” embraces chemically, enzymatically, or metabolically modified forms.

In some embodiments of the invention, the nucleic acid molecule includes the nucleotide sequence set forth in SEQ ID NO: 77, SEQ ID NO: 78, SEQ ID NO: 79 or SEQ ID NO: 156.

It would be understood by a person skilled in the art that any nucleotide sequence which encodes a chimeric antigen receptor having the amino acid sequence set forth in in SEQ ID NO: 6, SEQ ID NO: 7 or SEQ ID NO: 8, or a functional variant of in SEQ ID NO: 6, SEQ ID NO: 7 or SEQ ID NO: 8, is contemplated by the present invention. For example, variants of SEQ ID NO: 6, SEQ ID NO: 7 or SEQ ID NO: 8, are contemplated which comprise one or more different nucleic acids to SEQ ID NO: 77, SEQ ID NO: 78, SEQ ID NO: 79 or SEQ ID NO: 156 but which still encode identical amino acid sequences. Because of the degeneracy of the genetic code, a large number of nucleic acids can encode any given protein. For instance, the codons GCA, GCC, GCG and GCU all encode the amino acid alanine. Therefore, at every position in SEQ ID NO: 6, SEQ ID NO: 7 or SEQ ID NO: 8 where an alanine is specified by a codon, the codon can be altered to any of the corresponding codons described without altering the encoded polypeptide. Accordingly, every nucleotide sequence herein which encodes a chimeric antigen receptor having the amino acid sequence set forth in SEQ ID NO: 6, SEQ ID NO: 7 or SEQ ID NO: 8, or a functional variant of SEQ ID NO: 6, SEQ ID NO: 7 or SEQ ID NO: 8 also describes every possible silent variation of the nucleotide sequence. One of skill will recognise that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleotide sequence that encodes a polypeptide is implicit in each described sequence.

In another aspect, the present invention provides a nucleic acid construct including a nucleic acid molecule according to the invention. The nucleic acid construct may further comprise one or more of: an origin of replication for one or more hosts; a selectable marker gene which is active in one or more hosts; and/or one or more transcriptional control sequences.

As used herein, the term “selectable marker gene” includes any gene that confers a phenotype on a cell in which it is expressed, to facilitate the identification and/or selection of cells which are transfected or transformed with the construct.

“Selectable marker genes” include any nucleotide sequences which, when expressed by a cell transformed with the construct, confer a phenotype on the cell that facilitates the identification and/or selection of these transformed cells. A range of nucleotide sequences encoding suitable selectable markers are known in the art (for example Mortesen, R M. and Kingston R E. Curr Protoc Mol Biol, 2009; Unit 9.5). Exemplary nucleotide sequences that encode selectable markers include: Adenosine deaminase (ADA) gene; Cytosine deaminase (CDA) gene; Dihydrofolate reductase (DHFR) gene; Histidinol dehydrogenase (hisD) gene; Puromycin-N-acetyl transferase (PAC) gene; Thymidine kinase (TK) gene; Xanthine-guanine phosphoribosyltransferase (XGPRT) gene or antibiotic resistance genes such as ampicillin-resistance genes, puromycin-resistance genes, Bleomycin-resistance genes, hygromycin-resistance genes, kanamycin-resistance genes and ampicillin-resistance gene; fluorescent reporter genes such as the green, red, yellow or blue fluorescent protein-encoding genes; and luminescence-based reporter genes such as the luciferase gene, amongst others which permit optical selection of cells using techniques such as Fluorescence-Activated Cell Sorting (FACS).

Furthermore, it should be noted that the selectable marker gene may be a distinct open reading frame in the construct or may be expressed as a fusion protein with another polypeptide (e.g. the CAR).

As set out above, the nucleic acid construct may also comprise one or more transcriptional control sequences. The term “transcriptional control sequence” should be understood to include any nucleic acid sequence which effects the transcription of an operably connected nucleic acid. A transcriptional control sequence may include, for example, a leader, polyadenylation sequence, promoter, enhancer or upstream activating sequence, and transcription terminator. Typically, a transcriptional control sequence at least includes a promoter. The term “promoter” as used herein, describes any nucleic acid which confers, activates or enhances expression of a nucleic acid in a cell.

In some embodiments, at least one transcriptional control sequence is operably connected to the nucleic acid molecule of the invention. For the purposes of the present specification, a transcriptional control sequence is regarded as “operably connected” to a given nucleic acid molecule when the transcriptional control sequence is able to promote, inhibit or otherwise modulate the transcription of the nucleic acid molecule. Therefore, in some embodiments, the nucleic acid molecule is under the control of a transcription control sequence, such as a constitutive promoter or an inducible promoter.

The “nucleic acid construct” may be in any suitable form, such as in the form of a plasmid, phage, transposon, cosmid, chromosome, vector, etc., which is capable of replication when associated with the proper control elements and which can transfer gene sequences, contained within the construct, between cells. Thus, the term includes cloning and expression vehicles, as well as viral vectors. In some embodiments, the nucleic acid construct is a vector. In some embodiments the vector is a viral vector.

A promoter may regulate the expression of an operably connected nucleic acid molecule constitutively, or differentially, with respect to the cell, tissue, or organ at which expression occurs. As such, the promoter may include, for example, a constitutive promoter, or an inducible promoter. A “constitutive promoter” is a promoter that is active under most environmental and physiological conditions. An “inducible promoter” is a promoter that is active under specific environmental or physiological conditions. The present invention contemplates the use of any promoter which is active in a cell of interest. As such, a wide array of promoters would be readily ascertained by one of ordinary skill in the art.

Mammalian constitutive promoters may include, but are not limited to, Simian virus 40 (SV40), cytomegalovirus (CMV), P-actin, Ubiquitin C (UBC), elongation factor-1 alpha (EF1A), phosphoglycerate kinase (PGK) and CMV early enhancer/chicken β actin (CAGG).

Inducible promoters may include, but are not limited to, chemically inducible promoters and physically inducible promoters. Chemically inducible promoters include promoters which have activity that is regulated by chemical compounds such as alcohols, antibiotics, steroids, metal ions or other compounds. Examples of chemically inducible promoters include: tetracycline regulated promoters (e.g. see U.S. Pat. Nos. 5,851,796 and 5,464,758); steroid responsive promoters such as glucocorticoid receptor promoters (e.g. see U.S. Pat. No. 5,512,483), ecdysone receptor promoters (e.g. see U.S. Pat. No. 6,379,945) and the like; and metal-responsive promoters such as metallothionein promoters (e.g. see U.S. Pat. Nos. 4,940,661, 4,579,821 and 4,601,978) amongst others.

As mentioned above, the control sequences may also include a terminator. The term “terminator” refers to a DNA sequence at the end of a transcriptional unit which signals termination of transcription. Terminators are 3′-non-translated DNA sequences generally containing a polyadenylation signal, which facilitate the addition of polyadenylate sequences to the 3′-end of a primary transcript. As with promoter sequences, the terminator may be any terminator sequence which is operable in the cells, tissues or organs in which it is intended to be used. Suitable terminators would be known to a person skilled in the art.

As will be understood, the nucleic acid construct of the invention can further include additional sequences, for example sequences that permit enhanced expression, cytoplasmic or membrane transportation, and location signals. Specific non-limiting examples include an Internal Ribosome Entry Site (IRES).

The present invention extends to all genetic constructs essentially as described herein. These constructs may further include nucleotide sequences intended for the maintenance and/or replication of the genetic construct in eukaryotes and/or the integration of the genetic construct or a part thereof into the genome of a eukaryotic cell.

Methods are known in the art for the deliberate introduction (transfection/transduction) of exogenous genetic material, such as the nucleic acid construct of the present invention, into eukaryotic cells. As will be understood the method best suited for introducing the nucleic acid construct into the desired host cell is dependent on many factors, such as the size of the nucleic acid construct, the type of host cell the desired rate of efficiency of the transfection/transduction and the final desired, or required, viability of the transfected/transduced cells. Non-limiting examples of such methods include; chemical transfection with chemicals such as cationic polymers, calcium phosphate, or structures such as liposomes and dendrimers; non-chemical methods such as electroporation, sonoporations, heat-shock or optical transfection; particle-based methods such as ‘gene gun’ delivery, magnetofection, or impalefection or viral transduction.

The nucleic acid construct will be selected depending on the desired method of transfection/transduction. In some embodiments of the invention, the nucleic acid construct is a viral vector, and the method for introducing the nucleic acid construct into a host cell is viral transduction. Methods are known in the art for utilising viral transduction to elicit expression of a CAR in a PBMC (Parker, L L. et al. Hum Gene Ther. 2000; 11: 2377-87) and more generally utilising retroviral or lentiviral systems for transduction of mammalian cells (Barde et al, “Production and Titration of Lentiviral Vectors,” Current Protocols in Neuroscience, Volume 53, Issue 1 (2010) (also cited as Current Protocols in Neuroscience 4.21.1-4.21.23 (October 2010) and Cepko, C. and Pear, W. Curr Protoc Mol Biol. 2001, unit 9.9). In other embodiments, the nucleic acid construct is a plasmid, a cosmid, an artificial chromosome or the like, and can be transfected into the cell by any suitable method known in the art.

### Modified Cells

In another aspect, the invention provides a genetically modified cell that includes the chimeric antigen receptor of the invention.

In another aspect, the invention provides a genetically modified cell that includes the nucleic acid molecule of the invention, or includes the nucleic acid construct of the invention, or a genomically integrated form of the nucleic acid construct.

In another aspect, the present invention provides a method of generating a genetically modified cell, said method comprising transducing the cell, preferably an immune cell, with a nucleic acid construct encoding a CAR of the invention as described herein, so that the transduced cell expresses the CAR, thereby generating a genetically modified cell.

As referred to herein, a “genetically modified cell” includes any cell comprising a non-naturally occurring and/or introduced nucleic acid molecule or nucleic acid construct encompassed by the present invention. The introduced nucleic acid molecule or nucleic acid construct may be maintained in the cell as a discreet DNA molecule, or it may be integrated into the genomic DNA of the cell.

Genomic DNA of a cell should be understood in its broadest context to include any and all endogenous DNA that makes up the genetic complement of a cell. As such, the genomic DNA of a cell should be understood to include chromosomes, mitochondrial DNA and the like. As such, the term “genomically integrated” contemplates chromosomal integration, mitochondrial DNA integration, and the like. The “genomically integrated form” of the construct may be all or part of the construct. However, in some embodiments the genomically integrated form of the construct at least includes the nucleic acid molecule of the invention.

In some embodiments the activation receptor (from which a portion of signalling domain is derived) is the CD3 co-receptor complex.

In some embodiments, the co-stimulatory receptor (from which a portion of signalling domain is derived) is selected from the group consisting of CD28 or 4-1 BB (CD137).

As described above, a cellular immune response is typically only induced when an activation signal (typically in response to an antigen) and a co-stimulation signal are simultaneously experienced. Therefore, by having a genetically modified cell in accordance with some of the above embodiments, which provides both an intracellular activation signal and an intracellular co-stimulation signal, ensures that a sufficient immune response can be induce in response to the recognition by the CAR(s) of their cognate antigen.

In some embodiments, the genetically modified cell is further modified so as to constitutively express co-stimulatory receptors. In further embodiments, the genetically modified cell is further modified so as to express ligands for the co-stimulatory receptors, thereby facilitating auto-stimulation of the cell. Examples of CAR-expressing T cells that also express both co-stimulatory receptors and their cognate ligands (so as to induce auto-stimulation) are known in the art and include, inter alia, those disclosed in Stephen M T. et al. Nat Med, 2007; 13: 1440-9.

The potency of a genetically modified cell including a CAR can be enhanced by further modifying the cell so as to secrete cytokines, preferably proinflammatory or proproliferative cytokines. This secretion of cytokines provide both autocrine support for the cell expressing the CAR, and alters the local environment surrounding the CAR-expressing cell such that other cells of the immune system are recruited and activated. Consequently, in some embodiments of the invention the genetically modified cell is further modified to secret cytokines. This secretion may be constitutive, or may be inducible upon recognition of a CAR of its cognate antigen of ligand.

Whilst any one or more cytokines can be selected depending on the desired immune response, preferable cytokines include IL-2, IL-7, IL-12, IL-15, IL-17 and IL-21, or a combination thereof.

The genetically modified cell can be any suitable immune cell, or can be a homogeneous or a heterogeneous cell population. In some embodiments, the cell is an immune cell such as a leukocyte. In some embodiments, the cell is a Peripheral Blood Mononuclear Cell (PBMC), a lymphocyte, a T cell (including a CD4+ T cell or a CD8+ T cell), a natural killer (NK) cell, a natural killer T cell or a tumour infiltrating lymphocyte (TIL).

In a preferred embodiment, the immune cell that expresses the CAR is a T cell. Illustrative examples of suitable T cells include helper T cells (HTL; CD4⁺ T cell), a cytotoxic T cell (CTL; CD8⁺ T cell), CD4⁺CD8⁺ T cell, CD4⁻CD8⁻ T cell, or any other subset of T cells. Other illustrative examples of suitable T cells include T cells expressing one or more of the following markers: CD3, CD4, CD8, CD27, CD28, CD45RA, CD45RO, CD62L, CD127, CD197, and HLA-DR.

Upon creation of a genetically modified cell of the invention it may be desirable to expand the cell population in vitro to increase the total cell numbers available for use in treatment. This can be done using the step of exposing the cell to an antigen for the CAR. Accordingly, in another aspect the present invention provides a method of expanding in vitro the genetically modified cell of the invention, the method including the step of exposing the cell to an antigen for the CAR. In some embodiments, the method includes the further step of exposing the cell to a cytokine.

In another aspect, the present invention provides a method of expanding in vitro the genetically modified cell of the invention, the method including the step of exposing the cell to an antigen for the CAR and simultaneously exposing the cell to a cytokine.

Preferable cytokines used include members of the IL-2 subfamily, the interferon subfamily, the IL-10 subfamily, the IL-1 subfamily, the IL-7 subfamily, the IL-15 subfamily, the IL-17 subfamily or the TGF-β subfamily. In some embodiments of the invention, the cytokine is selected from the group consisting of IFN-γ, IL-2, IL-5, IL-7, IL-8, IL-10, IL-12, IL-13, IL-15, IL-17, IL-18, TNF-α, TGF-β1, TGF-β2, TGF-β3 and GM-CSF, or a combination thereof. More preferred cytokines include IL-7 and IL-15.

In another aspect, the present invention provides a method of expanding in vitro the genetically modified cell of the invention, the method including exposing the cell to immobilised CD3 and CD28 agonists and contacting the cells with a media optimised for the cultivation of human T cells. In some embodiments of the invention, the agonists are immobilised on a beaded substrate (for example “MACS GMP” TransAct™ or “Human Activator” Dynabeads™). In some embodiments of the invention, the antibodies are immobilised on an alternative surface such as the surface of a tissue culture vessel, a culture flask, plate or bioreactor.

In some embodiments the CD3 and CD28 agonists are anti-CD3 and anti-CD28 antibodies.

In some embodiments the genetically modified cell is exposed to immobilised CD3 and CD28 agonists for about 1 day or about 2 days and transduced the following day. In some embodiments the transduction is performed using a viral vector, for example a lentiviral or retroviral vector. In some embodiments the cell is contacted with a media for about 1 day, about 2 days, about 3 days, about 4 days, about 5 days, about 6 days, about 7 days, about 8 days, about 9 days, about 10 days, or more than 10 days. In some embodiments the cell is contacted with a media for about 7 to about 10 days.

In some embodiments the media is TexMACS™ GMP media. In some embodiments the media supplemented with cytokines. Preferable cytokines used include members of the IL-2 subfamily, the interferon subfamily, the IL-10 subfamily, the IL-1 subfamily, the IL-7 subfamily, the IL-15 subfamily, the IL-17 subfamily or the TGF-6 subfamily. In some embodiments of the invention, the cytokine is selected from the group consisting of IFN-γ, IL-2, IL-5, IL-7, IL-8, IL-10, IL-12, IL-13, IL-15, IL-17, IL-18, TNF-α, TGF-β1, TGF-β2, TGF-β3 and GM-CSF, or a combination thereof. More preferred cytokines include IL-7 and IL-15.

As would be understood by a person skilled in the art, depending on the signalling domain of the CAR, recognition by the CAR of its cognate antigen will lead to intracellular signalling that may ultimately lead to cellular proliferation. Accordingly, small numbers of cells, or even individual cells, can be expanded (or in the case of a single cell, clonally expanded) to form therapeutically significant numbers. This process can be further enhanced by the provision of cytokines.

### Methods

In another aspect, the present invention provides a method of killing a cell expressing mesothelin, the method including exposing the cell expressing a mesothelin to a genetically modified cell of the invention, thereby killing the cell.

Therefore, in some embodiments the invention, the CAR directly recognises mesothelin. In other embodiments, the CAR indirectly recognises mesothelin.

As used herein the term “directly recognises” includes direct binding of the antigen-recognition domain of the CAR to the mesothelin, or an epitope thereof. In another non-limiting example, the antigen-recognition domain may directly bind to a processed form of mesothelin, which may be presented by antigen presenting molecules such as the major histocompatibility complex (MHC).

As an alternative to the CAR directly recognising a cell expressing or displaying mesothelin, or an epitope thereof, the CAR may be directed against a cell expressing or displaying mesothelin, or an epitope thereof, by an indirect means.

Consequently, in some embodiments of the invention, the chimeric antigen receptor recognises mesothelin via an intermediate. An intermediate may be a molecule such as a probe that binds or interacts directly with mesothelin. Non-limiting examples of such probes include antibodies, a Fab of an antibody, a scFv, a soluble engineered TCR or an aptamer. The CAR may be able to directly recognise the probe or the probe may have a tag that is recognised by the CAR. In either regard the probe provides the specificity for the target cell (namely a cell expressing or displaying mesothelin or an epitope thereof) whilst the genetically modified cell having the CAR provides the efficacy and directs an immune response against the target cell. Alternatively, the intermediate could be a cell endogenous marker which is associated with, or its expression is correlated to, mesothelin. The dysregulation of the marker may be a cause of or a result of mesothelin.

In some embodiments of the invention, the method of killing a cell expressing or displaying mesothelin or an epitope thereof further includes the step of exposing the cell expressing or displaying mesothelin or an epitope thereof to an intermediate.

In some embodiments of the invention, the intermediate is a probe that binds to mesothelin or an epitope thereof and the chimeric antigen receptor recognises the probe. Preferably the probe is an antibody or an aptamer.

The term “aptamer” as used throughout the specification refers to any oligonucleic acid, polynucleic acid, peptide or polypeptide which specifically binds to, or preferentially forms a complex with, a target (specifically mesothelin).

In some embodiments of the invention, the probe includes a tag and the chimeric antigen receptor recognises the tag. Examples of a CAR that recognise cells by way of an intermediate are known in the art, for example European patent application EP 2651442.

In some embodiments of the invention, the cell expressing or displaying mesothelin, or epitope thereof, is within the body of a subject. In some embodiments, the subject is a human. In some embodiments, the method further includes exposing the cell expressing or displaying mesothelin, or an epitope thereof, to a genetically modified together with an exogenous cytokine.

In some embodiments of the invention, the genetically modified cell is a genetically modified cell autologous to the cell expressing or displaying mesothelin, or epitope thereof, from the subject.

In another aspect, the present invention provides a method of treating or preventing cancer in a subject, the method including providing a subject with a genetically modified cell of the invention, thereby treating or preventing cancer.

The terms “treat”, “treating” or “treatment,” as used herein are to be understood to include within their scope one or more of the following outcomes: (i) inhibiting to some extent the growth of a primary tumour in a subject, including, slowing down and complete growth arrest, and including reducing the growth of the primary tumour after resection; (ii) inhibiting to some extent the growth and formation of one or more secondary tumours in a subject; (iii) reducing the number of tumour cells in a subject; (iv) reducing the size of a tumour in the subject; (v) inhibiting (i.e. reduction, slowing down or complete stopping) of tumour cell infiltration into peripheral organs; (vi) inhibiting (i.e. reduction, slowing down or complete stopping) of metastasis; (vii) improving the life expectancy of a subject as compared to the untreated state; (viii) improving the quality of life of a subject as compared to the untreated state; (ix) alleviating, abating or ameliorating at least one symptom of cancer in a subject; (x) causing regression or remission of cancer in a subject; (xi) relieving a condition in a subject that is caused by cancer; and (xii) stopping symptoms in a subject that are associated with cancer.

The terms “prevent” or “preventing” as used herein are to be understood to include within their scope inhibiting the formation of a primary tumour in a subject, inhibiting the formation of one or more secondary tumours in a subject, or reducing or eliminating the recurrence of cancer in a subject in remission.

The term “inhibiting” as used herein is taken to mean a decrease or reduction in the growth of a cancer, cancerous cell or tumour when compared to the growth in a control, such as an untreated cell or subject. In some embodiments, growth may be decreased or reduced by at least 10%, 20%, 30%, 40%, 50%, 60%, 70%, 80%, 90%, or 100%, relative to an untreated control.

Inhibition of the growth of a cancer, tumour or cancerous cell may be assessed by a range of methods known in the art. For example, for a cancerous cell in vitro, the growth of the cell may be determined by a suitable proliferation assay, or by method which assess the extent of incorporation of tritiated thymidine into cellular DNA over a given period of time. For a tumour or cancerous cell present in vivo, the growth of the tumour or cell may be determined for example by a suitable imaging method known in the art.

The term “subject” as used herein refers to any animal capable of suffering from cancer. Particular subjects of interest are human beings, and scientifically relevant species such as mice, rats, ferrets, guinea pigs, hamsters, non-human primates, dogs, pigs and sheep, or economically relevant animals such as horses, dogs, cats and cattle. In a preferred embodiment of the invention, the subject is a human.

A reference to “providing a subject with” relates to administering to the subject the genetically modified cell. Alternatively, the genetically modified cell may be generated within the subject. For example, the genetically modified cell may be generated in vivo such that the subject has an endogenous population of genetically modified cells. Suitable means for such in vivo generation are known in the art and include gene therapy of a subject.

As used throughout the specification a reference to a CAR being “directed” against a target cell expressing or displaying mesothelin, or an epitope thereof, contemplates the selective targeting of an immune response toward a cell based on the cell expressing or displaying mesothelin. Importantly, such targeting is not limited to direct recognition of mesothelin by a CAR. That is to say that the CAR itself does not have to directly recognise or bind to mesothelin but merely has to be able to selectively recognise and be activated by a cell that expresses mesothelin.

Therefore, in some embodiments of the invention, the CAR directly recognises mesothelin. In other embodiments, the CAR indirectly recognises the mesothelin.

As used herein the term “directly recognises” includes direct binding of the antigen-recognition domain of the CAR to mesothelin, or an epitope thereof, when the molecule is present in its natural form. In another non-limiting example, the antigen-recognition domain may directly bind to a processed form of mesothelin, which may be presented by antigen presenting molecules such as the major histocompatibility complex (MHC).

Whilst the provision of a genetically modified cell expressing a CAR directed against a target cell expressing or displaying mesothelin may be sufficient to provide effective immunotherapy against precancerous or cancerous cells, the provision of adjuvants together with the genetically modified cells may further enhance the induction of the immune response and may augment the immunotherapy. Cytokines, preferably proinflammatory cytokines, are particularly suitable adjuvants for provision to a subject together with genetically modified cells having CARs.

Therefore, in some embodiments of the invention, the genetically modified cell is administered to the subject together with a cytokine. It is to be understood that as used throughout the specification the term “together with” includes the genetically modified cell being administered simultaneously with a cytokine or administered in combination with a cytokine. Consequently, when administered in combination with a cytokine this may be considered to include a combination therapy whereby a subject's immunotherapy includes both treatment with a cytokine and treatment with a genetically modified cell having a CAR directed against a target cell expressing or displaying mesothelin, or an epitope thereof. In some forms, the cytokine is administered on a different day (>24 hrs) to the administration of the genetically modified cells. In other forms the cytokine is administered on the same day (within 24 hrs) as the genetically modified cells. In further forms the cytokine(s) and the genetically modified cell is administered within 18 hrs, 12 hrs, 6 hrs, 4 hrs, 2 hrs, 1 hr, 45 mins, 30 mins, 15 mins, 10 mins, 5 mins, 2 mins or lmin of each other.

Suitable cytokines for administration together with the genetically modified cell include IL-2, IL-4, IL-6, IL-7, IL-9, IL-12, IL-15, IL-17, IL-18, IL-21, IL-23, IFNα, IFNβ, IFNγ, GM-CSF, TGFβ and TNFα. Preferred cytokines include IL-7 and IL-15. Furthermore, the cytokines may be administered as recombinant forms, natural forms, or via delivery systems such as fusions with proteins, delivered as a nucleic acid sequence which is expressed in the genetically modified cell or conjugated with a polymer such a polyethylene glycol (PEG).

The cell to be genetically modified can be obtained from any suitable source. In some embodiments of the invention the cell to be genetically modified is an autologous cell, being a cell autologous to the cell expressing or displaying mesothelin, or epitope thereof. Advantageously, an autologous cell would not be recognised as ‘non-self’ by the subject's immune system and would therefore be tolerated by the subject. However, in some forms of cancer suitable autologous cells may not be readily available. Therefore, in some embodiments of the invention the cell to be genetically modified is an allogeneic or heterologous cell.

### Cancer

The term “cancer” will be understood to include benign, pre-cancerous, pre-neoplastic or non-metastatic tumours or metastatic tumours. In some embodiments, the cancer is metastatic cancer, such as stage III or stage IV cancer.

Cancers particularly suited for prevention or treatment are those that express or overexpress mesothelin. The expression or overexpression of mesothelin on a cancer or a specific tumour can be determined by detecting mesothelin protein present in or on a cancer cell or detecting RNA (e.g. mRNA) encoding mesothelin that is expressed by a cancer cell.

In some embodiments, the type of cancer to be treated includes those having a benign, pre-cancerous, pre-neoplastic or non-metastatic tumour. A benign tumour will be understood to not be a malignant tumour and to not invade nearby tissue or spread to other parts of the body. Similarly non-metastatic cancer will be understood to not invade nearby tissue or spread to other parts of the body. “Pre-cancerous” or “pre-neoplasia” generally refers to a condition or a growth that typically precedes or develops into a cancer. A “pre-cancerous” growth may have cells that are characterized by abnormal cell cycle regulation, proliferation, or differentiation, which can be determined by markers of cell cycle.

In one embodiment, the cancer is a secondary cancer or metastases. The secondary cancer may be located in any organ or tissue, and particularly those organs or tissues having relatively higher hemodynamic pressures, such as lung, liver, kidney, pancreas, bowel and brain. The secondary cancer may be detected in the ascites fluid and/or lymph nodes.

In any aspect or embodiment, the subject is an adult.

In any aspect or embodiment, the subject has a mesothelin positive cancer such as mesothelioma, lung, pancreatic, ovarian, gastric, triple negative breast, thymic, endometrial, colorectal, cervical, esophageal or bile duct cancer. In one embodiment, the cancer may be refractory or in second or later relapse.

Pre-neoplastic, neoplastic and metastatic cancers are particular examples to which the methods of the invention may be applied. Broad examples include breast tumours, colorectal tumours, adenocarcinomas, mesothelioma, bladder tumours, prostate tumours, germ cell tumour, hepatoma/cholongio, carcinoma, neuroendocrine tumours, pituitary neoplasm, small round cell tumour, squamous cell cancer, melanoma, atypical fibroxanthoma, seminomas, nonseminomas, stromal leydig cell tumours, Sertoli cell tumours, skin tumours, kidney tumours, testicular tumours, brain tumours, ovarian tumours, stomach tumours, pancreatic tumours, oral tumours, bladder tumours, bone tumours, cervical tumours, esophageal tumours, laryngeal tumours, liver tumours, lung tumours, vaginal tumours and Wilm's tumour. Preferred examples include pancreatic tumours, ovarian tumours, stomach tumours, lung tumours, liver tumours, colorectal tumours, cervical tumours, endometrial tumours, renal tumours, breast tumours, and testicular tumours.

Examples of particular cancers include but are not limited to adenocarcinoma, adenoma, adenofibroma, adenolymphoma, adontoma, AIDS related cancers, acoustic neuroma, acute lymphocytic leukemia, acute myeloid leukemia, adenocystic carcinoma, adrenocortical cancer, agnogenic myeloid metaplasia, alopecia, alveolar soft-part sarcoma, ameloblastoma, angiokeratoma, angiolymphoid hyperplasia with eosinophilia, angioma sclerosing, angiomatosis, apudoma, anal cancer, angiosarcoma, aplastic anaemia, astrocytoma, ataxia-telangiectasia, basal cell carcinoma (skin), bladder cancer, bone cancers, bowel cancer, brain stem glioma, brain and CNS tumours, breast cancer (preferably triple negative breast cancer), branchioma, CNS tumours, carcinoid tumours, cervical cancer, childhood brain tumours, childhood cancer, childhood leukemia, childhood soft tissue sarcoma, chondrosarcoma, choriocarcinoma, chronic lymphocytic leukemia, chronic myeloid leukemia, colorectal cancers, cutaneous T-cell lymphoma, carcinoma (e.g. Walker, basal cell, basosquamous, Brown-Pearce, ductal, Ehrlich tumour, Krebs 2, Merkel cell, mucinous, non-small cell lung, oat cell, papillary, scirrhous, bronchiolar, bronchogenic, squamous cell, and transitional cell), carcinosarcoma, cervical dysplasia, cystosarcoma phyllodies, cementoma, chordoma, choristoma, chondrosarcoma, chondroblastoma, craniopharyngioma, cholangioma, cholangiocarincoma, cholesteatoma, cylindroma, cystadenocarcinoma, cystadenoma, dermatofibrosarcoma-protuberans, desmoplastic-small-round-cell-tumour, ductal carcinoma, dysgerminoam, endocrine cancers, endometrial cancer, ependymoma, esophageal cancer, Ewing's sarcoma, extra-hepatic bile duct cancer, eye cancer, eye: melanoma, retinoblastoma, fallopian tube cancer, fanconi anaemia, fibroma, fibrosarcoma, gall bladder cancer, gastric cancer, gastrointestinal cancers, gastrointestinal-carcinoid-tumour, genitourinary cancers, germ cell tumours, gestationaltrophoblastic-disease, glioma, gynaecological cancers, giant cell tumours, ganglioneuroma, glioma, glomangioma, granulosa cell tumour, gynandroblastoma, haematological malignancies, hairy cell leukemia, head and neck cancer, hepatocellular cancer, hereditary breast cancer, histiocytosis, Hodgkin's disease, human papillomavirus, hydatidiform mole, hypercalcemia, hypopharynx cancer, hamartoma, hemangioendothelioma, hemangioma, hemangiopericytoma, hemangiosarcoma, hemangiosarcoma, histiocytic disorders, histiocytosis malignant, histiocytoma, hepatoma, hidradenoma, hondrosarcoma, immunoproliferative small, opoma, ontraocular melanoma, islet cell cancer, Kaposi's sarcoma, kidney cancer, langerhan's cell-histiocytosis, laryngeal cancer, leiomyosarcoma, leukemia, li-fraumeni syndrome, lip cancer, liposarcoma, liver cancer, lung cancer, lymphedema, lymphoma, Hodgkin's lymphoma, non-Hodgkin's lymphoma, leigomyosarcoma, leukemia (e.g. B-cell, mixed cell, null-cell, T-cell, T-cell chronic, HTLV-Ilassociated, lymphangiosarcoma, lymphocytic acute, lymphocytic chronic, mast-cell and myeloid), leukosarcoma, leydig cell tumour, liposarcoma, leiomyoma, leiomyosarcoma, lymphangioma, lymphangiocytoma, lymphagioma, lymphagiomyoma, lymphangiosarcoma, male breast cancer, malignant-rhabdoid-tumour-of-kidney, medulloblastoma, melanoma, Merkel cell cancer, mesothelioma, metastatic cancer, mouth cancer, multiple endocrine neoplasia, mycosis fungoides, myelodysplastic syndromes, myeloma, myeloproliferative disorders, malignant carcinoid syndrome carcinoid heart disease, medulloblastoma, meningioma, melanoma, mesenchymoma, mesonephroma, mesothelioma, myoblastoma, myoma, myosarcoma, myxoma, myxosarcoma, nasal cancer, nasopharyngeal cancer, nephroblastoma, neuroblastoma, neurofibromatosis, Nijmegen breakage syndrome, non-melanoma skin cancer, non-small-cell-lung-cancer-(nscic), neurilemmoma, neuroblastoma, neuroepithelioma, neurofibromatosis, neurofibroma, neuroma, neoplasms (e.g. bone, breast, digestive system, colorectal, liver), ocular cancers, oesophageal cancer, oral cavity cancer, oropharynx cancer, osteosarcoma, ostomy ovarian cancer, pancreas cancer, paranasal cancer, parathyroid cancer, parotid gland cancer, penile cancer, peripheral-neuroectodermal-tumours, pituitary cancer, polycythemia vera, prostate cancer, osteoma, osteosarcoma, ovarian carcinoma, papilloma, paraganglioma, paraganglioma nonchromaffin, pinealoma, plasmacytoma, protooncogene, rare-cancers-and-associated-disorders, renal cell carcinoma, retinoblastoma, rhabdomyosarcoma, Rothmund-Thomson syndrome, reticuloendotheliosis, rhabdomyoma, salivary gland cancer, sarcoma, schwannoma, Sezary syndrome, skin cancer, small cell lung cancer (scic), small intestine cancer, soft tissue sarcoma, spinal cord tumours, squamous-cell-carcinoma-(skin), stomach cancer, synovial sarcoma, sarcoma (e.g. Ewing's experimental, Kaposi's and mast-cell sarcomas), Sertoli cell tumour, synovioma, testicular cancer, thymus cancer, thyroid cancer, transitional-cell-cancer-(bladder), transitional-cell-cancer-(renal-pelvis-/-ureter), trophoblastic cancer, teratoma, theca cell tumour, thymoma, trophoblastic tumour, urethral cancer, urinary system cancer, uroplakins, uterine sarcoma, uterus cancer, vaginal cancer, vulva cancer, Waldenstrom's-macroglobulinemia and Wilms' tumour. Preferred particular examples include pleural or peritoneal mesothelioma, gastric cancer, endometrial cancers, colorectal cancer, non-small cell lung adenocarcinoma, cholangiocarcinoma, ovarian carcinoma, esophageal cancer, pancreatic ductal adenocarcinoma, triple negative breast cancer and any other mesothelin-positive cancers.

In some embodiments of the invention the method is used for the prevention or treatment of a cancer selected from one or more of; brain cancer, oesophageal cancer, mouth cancer, tongue cancer, thyroid cancer, lung cancer, stomach cancer, pancreatic cancer, kidney cancer, colon cancer, rectal cancer, prostate cancer, bladder cancer, cervical cancer, epithelial cell cancers, skin cancer, leukaemia, lymphoma, myeloma, breast cancer, ovarian cancer, endometrial cancer, testicular cancer. Preferably the cancer is selected from one or more of lung cancer, oesophageal cancer, stomach cancer, colon cancer, prostate cancer, bladder cancer, cervical cancer, vaginal cancers, epithelial cell cancers, skin cancer, blood-related cancers, breast cancer, endometrial cancer, uterine cancer testicular cancer.

The existence of, improvement in, treatment of, or minimisation of progression of cancer may be determined by any clinically or biochemically relevant method as described herein or known in the art. A positive response to treatment or a minimisation of progression of a cancer may be determined by any method known in the art and may include the determination of:
a reduction in the number of cancer cells;
a reduction in the tumour size;
an inhibition (i.e., slow to some extent and preferably stop) of cancer cell infiltration into peripheral organs;
an inhibition (i.e., slow to some extent and preferably stop) of tumour metastasis;
a reduction or complete prevention of tumour metastasis following removal of the primary tumour;
an inhibition, to some extent, of tumour growth;
relieving, to some extent, of one or more of the symptoms associated with the cancer; and/or
increased survival of the subject.

The determination of any of the above may be considered to be a positive response to a treatment as described herein.

The subject who has received the treatment for cancer may be in partial or complete remission. In other words, the subject, having received a treatment for cancer, as described above, may have a 10%, 20%, 30%, 40%, 50%, 60%, 70%, 80%, 90% or greater reduction in the measurable parameters of tumour growth as may be found on physical examination, radiologic study, or by biomarker levels from a blood or urine test. Alternatively, where the subject is in complete remission, there is a complete disappearance of all detectable manifestations of disease, such that the subject does not have any detectable signs of cancer. The subject may have substantially undetectable signs of cancer. A cancer that is “substantially undetectable” generally refers to a circumstance where therapy has depleted the size, volume or other physical measure of a cancer so that using relevant standard detection techniques such as in vivo imaging, the cancer, as a consequence of the therapy, is not clearly detectable.

The objective or outcome of treatment may be to reduce the number of cancer cells; reduce the primary tumour size; inhibit (i.e., slow to some extent and preferably stop) cancer cell infiltration into peripheral organs; inhibit (i.e., slow to some extent and preferably stop) tumour metastasis; inhibit, to some extent, tumour growth; and/or relieve to some extent one or more of the symptoms associated with the disorder.

Efficacy of treatment can be measured by assessing the duration of survival, time to disease progression, the response rates (RR), duration of response, and/or quality of life.

In one embodiment, the method is particularly useful for delaying cancer progression. In one embodiment, the method is particularly useful for extending survival of the subject, including overall survival as well as progression free survival. It will be understood that overall survival is the length of time from either the date of diagnosis or the start of treatment of a cancer, that patients diagnosed with the cancer are still alive. It will be understood that progression free survival is the length of time during and after the treatment of a cancer that a patient lives with the disease but it does not get worse.

Survival analysis can be performed using well known techniques in the art including the Kaplan-Meier method. The Kaplan-Meier method estimates the survival function from life-time data. In medical research, it can be used to measure the fraction of patients living for a certain amount of time after treatment. A plot of the Kaplan-Meier method of the survival function is a series of horizontal steps of declining magnitude which, when a large enough sample is taken, approaches the true survival function for that population. The value of the survival function between successive distinct sampled observations (“clicks”) is assumed to be constant.

An important advantage of the Kaplan-Meier curve is that the method can take into account “censored” data-losses from the sample before the final outcome is observed (for instance, if a patient withdraws from a study). On the plot, small vertical tick-marks indicate losses, where patient data has been censored. When no truncation or censoring occurs, the Kaplan-Meier curve is equivalent to the empirical distribution.

In one embodiment, the method is particularly useful for providing a complete response to therapy whereby all signs of cancer in response to treatment have disappeared. This does not always mean the cancer has been cured. In one embodiment, the method is particularly useful for providing a partial response to therapy whereby there has been a decrease in the size of one or more tumours or lesions, or in the extent of cancer in the body, in response to treatment.

### Compositions and Administration

The delivery or administration of the genetically modified cell according to the invention may be delivery or administration of the cell alone, or delivery or administration of the cell formulated into a suitable pharmaceutical composition. Accordingly, the present invention provides a pharmaceutical composition including a genetically modified cell of the invention, and a pharmaceutically acceptable carrier.

Methods are known in the art for providing CAR-containing cells for immunotherapy (see for example Kershaw, M H. et al. Clin Cancer Res. 2006; 12(20): 6106-15; Parker L L. et al. Hum Gene Ther 2000; 11: 2337-87). Furthermore, protocols and methods are known in the art for the preparation, expansion and assessment of mammalian CAR-expressing cells (see for example Cheadle, E J. et al. Antibody Engineering: Methods and Protocols, Second Edition, Methods in Molecular Biology, vol. 907: 645-66) and are summarised in the Examples below.

The pharmaceutical composition may also include one or more pharmaceutically acceptable additives, including pharmaceutically acceptable salts, amino acids, polypeptides, polymers, solvents, buffers, excipients and bulking agents, taking into consideration the particular physical and chemical characteristics of the cell to be administered. In some embodiments, the pharmaceutical composition includes a suspension of genetically modified cells of the invention in a suitable medium, such as isotonic saline solution. In some embodiments, the pharmaceutical composition may include suitable adjuvants such as one or more cytokines as described above. In some embodiments, the pharmaceutical composition may also include an intermediate as described above.

Administration of the pharmaceutical composition may also be via parenteral means which include intravenous, intraventricular, intraperitoneal, intramuscular, intrapleurally or intracranial injection, or local injections to the site of a tumour or cancerous mass.

It will be understood that the invention disclosed and defined in this specification extends to all alternative combinations of two or more of the individual features mentioned or evident from the text or drawings. All of these different combinations constitute various alternative aspects of the invention.

### EXAMPLES

### Example 1—Anti-Mesothelin Antibody Characterisation

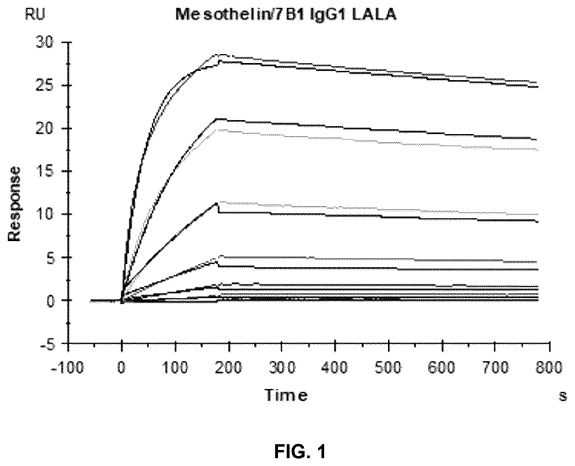

7B1 IgG1 LALA anti-mesothelin antibody was characterized using multi-cycle and single-cycle SPR experiments. FIG. 1 shows the sensorgrams of Mesothelin binding to 7B1 IgG1 LALA AA cAb7385-10.16 on FC3-1 (210805 experiment). Mesothelin concentrations 111 nM, 37.0 nM, 12.3 nM, 4.1 nM, 1.4 nM, 0.46 nM, 0.15 nM from top to bottom (higher concentrations were omitted).

The binding of mesothelin to 7B1 IgG1 LALA produced good fits with high affinities, K_{D} in the 0.6-0.9 nM range as described in the below table.

| | ka | SE(ka) | kd | SE (kd) | |
|---|---|---|---|---|---|
| Ligand | (1/Ms) | (1/Ms) | (1/s) | (1/s) | KD (M) |
| 7b1 IgG1 | 2.07E+05 | 2.42E+02 | 1.89−E04 | 1.50E−06 | 9.12E−10 |
| LALA - | | | | | |
| Sample 1 | | | | | |
| 7b1 IgG1 | 2.01E+05 | 1.77E+02 | 1.32E−04 | 6.41E−07 | 6.58E−10 |
| LALA - | | | | | |
| Sample 2 | | | | | |

### Example 2—CAR Design and Production

### CAR Construction

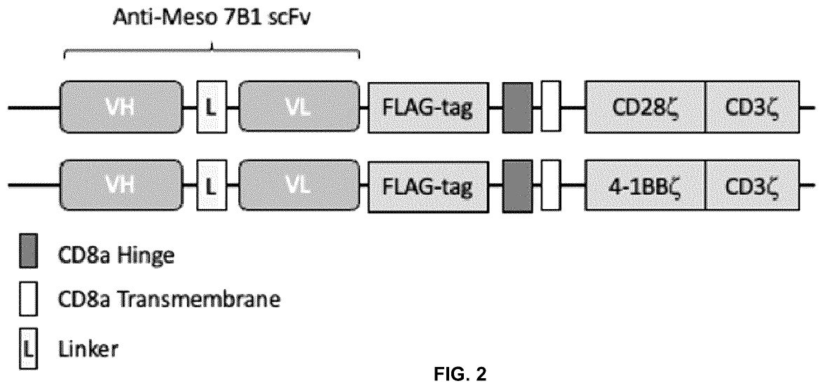

Two CAR genes were designed containing the 7B1 scFv linked to a FLAG sequence and the hinge and transmembrane domain of human CD8. This was linked to the cytoplasmic domains of either CD28 or CD137 (4-1 BB) together with the cytoplasmic domain of the chain of the CD3 complex, to generate two CARs: 7B1-2K and 7B1-BK (FIG. 2).

The genes were synthesized by Genscript and cloned into the Nhel/Eagl sites of the Addgene lentiviral plasmid, pCDH-EF1a-eFFly in place of the Fire-Fly luciferase gene.

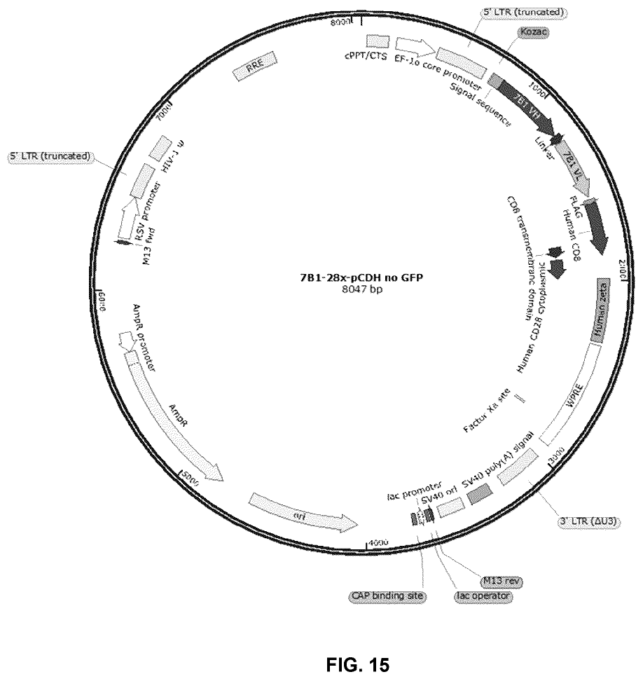

Lentiviral vector was produced as culture supernatant from HEK-293T cells transfected with four plasmids using Lipofectamine™ 3000: pCDH-EF1a-CAR (see FIG. 15 for plasmid map), pMDLg/pRRE, pRSV-Rev and pMD2.G, VSVG. Cells were cultured in Opti-MEM supplemented with 10% FBS and 100 mM Sodium Pyruvate. Lipofectamine™ 3000 Transfection Reagent (ThermoFischer Scientific) was used in accordance with the manufacturer's instructions. The Lentiviral vector was produced according to Origene's Lenti-vpak Packaging Kit Protocol. Viral supernatant was concentrated using Origene Lentivirus Concentrator and the titer initially determined on HCT116 cells.

### CAR T Cell Production

CAR T cells were generated by transduction using lentiviral vector according to Miltenyi Biotec's research CAR T cell workflow. Briefly, PBMCs were seeded at 2×10⁶ cells/mL in a 24 well plate. PBMC were activated the next day using Miltenyi TransAct for 2 days and transduced on Day 3, followed by culture with TexMACS media with IL-7 and IL-15 for 7-10 days.

Gene-specific primers were designed for 7B1-28K CARs spanning across the VH and VL portions of each scFv. PCR cycle number was compared to the housekeeping gene MCHR1 (copy number 2) and Viral copy number (VCN) in 7B1-28ζ transduced CAR T cells was performed using a digital drop PCR (ddPCR).

CAR T cell transductions and VCN calculations were consistent across T cells from several donors. The VCN per CAR T cell was equivalent with some variability between donors noted (Table 1)

TABLE 2
7B1-28ζ transduction efficiency in T cells from several donors

| | | | | | Copies | Copies |
|---|---|---|---|---|---|---|
| | | | | % Trans- | per | per CAR |
| Experiment | Buffy | MOI | Hits | duction | cell | T cell |
| BvSC_076 | 107 | Neat | 1 | 32.2 | 1.41 | 4.4 |
| BvSC_076 | 128 | 2:1 | 1 | 62 | 2.57 | 4.15 |
| BvSC_076 | 134 | 2:1 | 1 | 35.2 | 1.84 | 5.24 |
| BvSC_076 | 136 | 2:1 | 1 | 45.1 | 1.68 | 3.72 |
| BvSC_076 | 137 | 2:1 | 1 | 6.19 | 0.303 | 4.9 |
| BvSC_096 | 141 | 2:1 | 1 | 43.9 | 2.07 | 4.72 |
| BvSC_096 | 141 | 5:1 | 1 | 65.5 | 4.11 | 6.28 |

The VCN for various MOls was calculated. The MOI was varied (0.25, 0.5, 1, 2, 5) and the VCN monitored with 7B1-2ζ and the results displayed in Table 3 below.

TABLE 3
7B1-28ζ transduction efficiency in T cells from the same donor

| | | | | | Copies | Copies |
|---|---|---|---|---|---|---|
| | | | | % Trans- | per | per CAR |
| Experiment | Buffy | MOI | Hits | duction | cell | T cell |
| BvSC_096 | 141 | 5:1 | 1 | 65.5 | 4.11 | 6.28 |
| BvSC_096 | 141 | 2:1 | 1 | 43.9 | 2.07 | 4.72 |
| BvSC_096 | 141 | 1:1 | 1 | 27.2 | 1.39 | 5.11 |
| BvSC_096 | 141 | 0.5:1 | 1 | 15.2 | 0.81 | 5.3 |
| BvSC_096 | 141 | 0.25:! | 1 | 9.87 | 0.54 | 5.43 |

### Example 3—7B1 CAR T Domains Bind to Mesothelin and Anti-FLAG

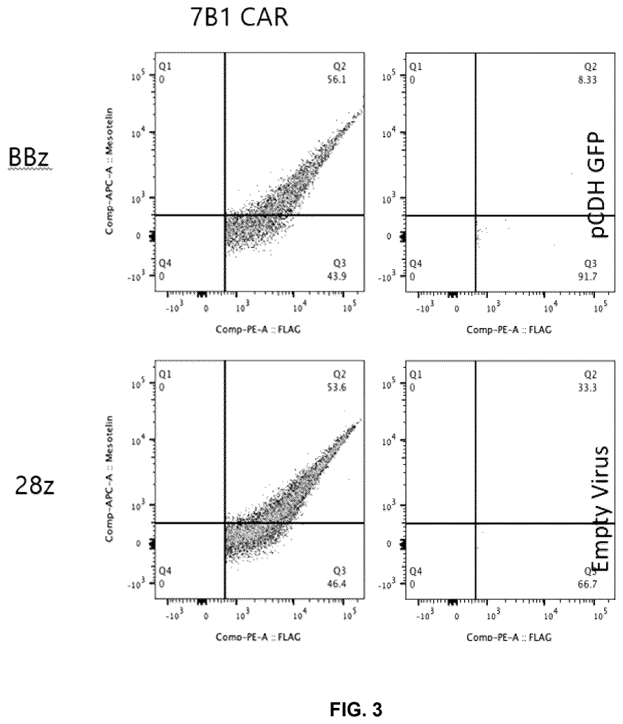

Anti-mesothelin CARs (7B1 scFv and CD28z, 41 BBz costimulatory domains) bind to their intended antigens, recombinant human mesothelin (Y axis) and anti-FLAG (X axis) (FIG. 3).

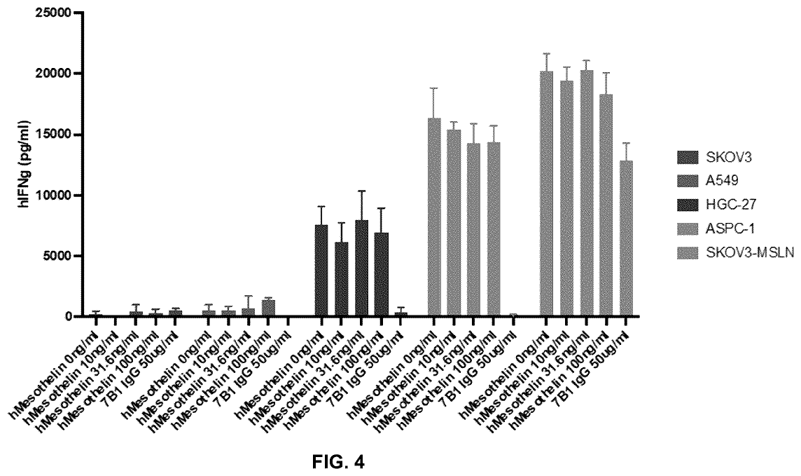

A dose response of soluble mesothelin (1-100 ng/mL) was added in combination with the anti-mesothelin CAR 7B1-28z and mesothelin negative cells (SKOV3 and A549) and positive cells with various levels of mesothelin (SKOV3-MSLN, ASPC-1, HGC-27). The soluble mesothelin at physiological levels had no effect on IFNg levels (FIG. 4). The 7B1 IgG CAR scFv was added as a competing antibody. Only at the highest dose of 50 ug/mL did the competing CAR antibody 7B1 decrease in IFNg (FIG. 4).

### Example 4—Cytokine Analysis of 7B1 CAR T Cells

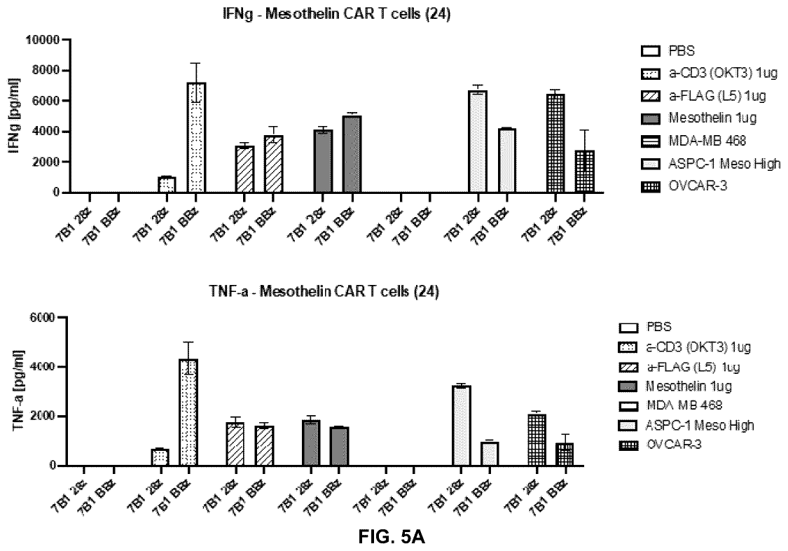

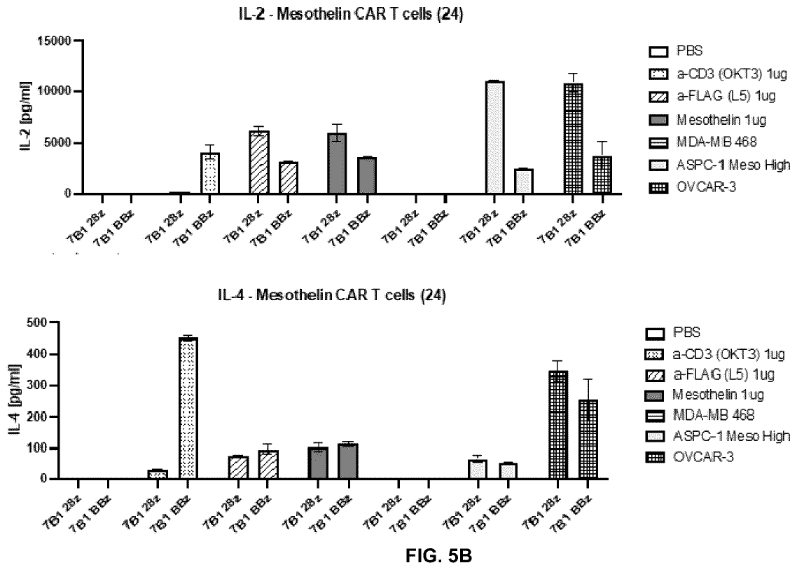

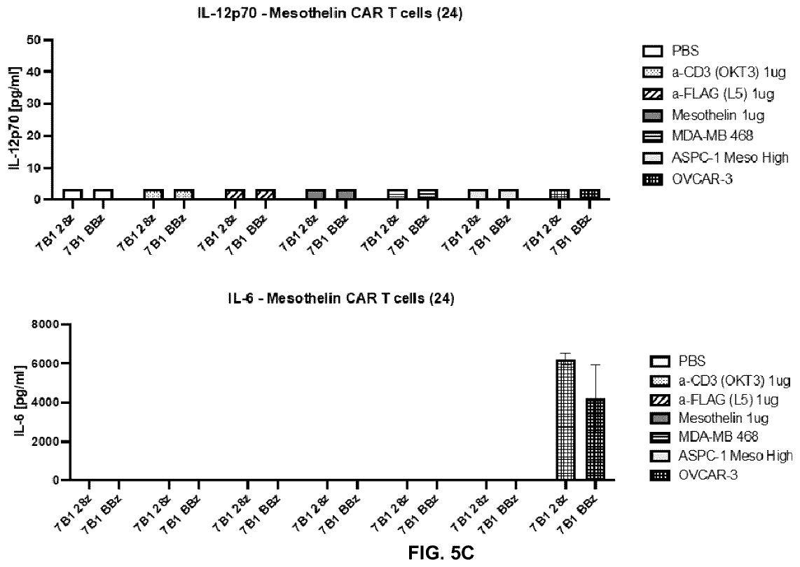

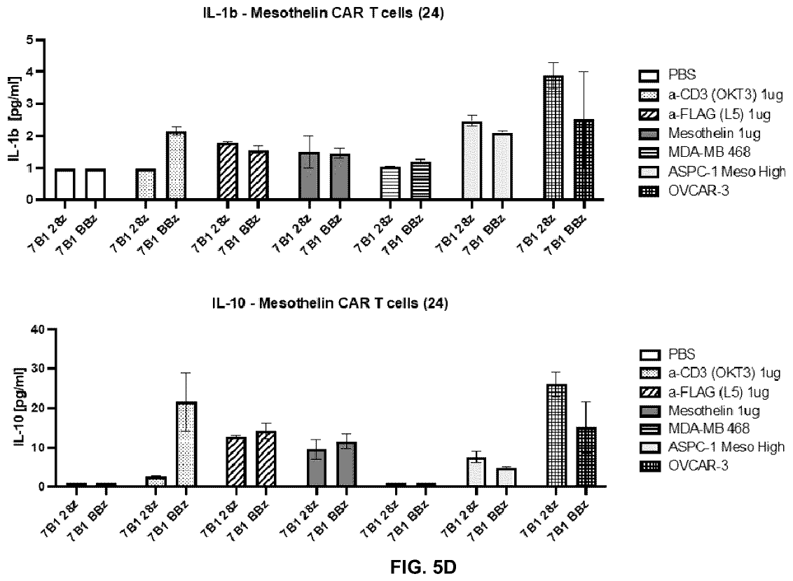

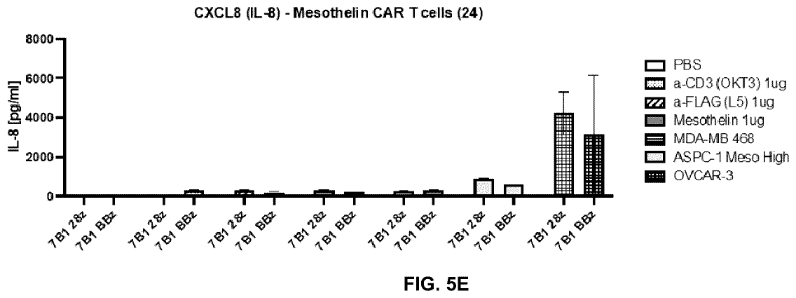

7B1 CD28z and 41 BB transduced T-cells were incubated with recombinant proteins (anti-CD3, anti-FLAG, mesothelin) and tumour cell lines (negative cell line MDA-MB-468 and positive cell lines ASPC1 and OVCAR3) for 24 hours before a panel of cytokines was measured. Cytokine secretion was determined using Legendplex. Specific increases in IFNg, TNFa, IL-2, IL-4 seen with 7B1 with both CD28z and 41 BBz co-stimulatory domains (FIG. 5).

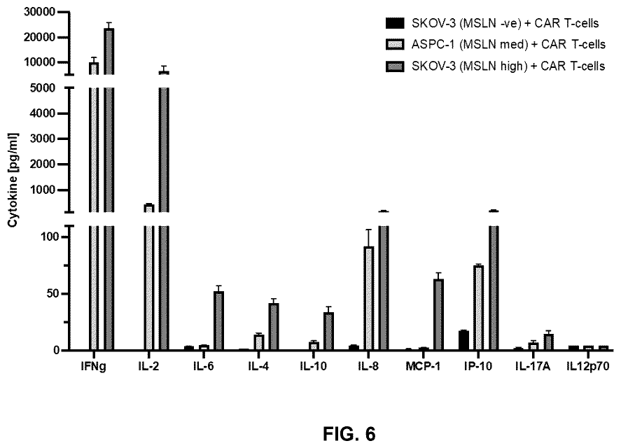

Cell lines with varying mesothelin expression were incubated with 7B1-28z CAR T-cells for 24 hours. ASPC1—medium expression of MSLN and SKOV-3 transfected cells with high expression of MSLN were evaluated and compared to MSLC negative cell line SKOV-3. Cytokines were measured by LegendPlex cytokine bead array. Cytokine release correlates with mesothelin expression for all cytokines. IFNg, IL-2 IL-8 and IL-10 upregulated with both medium and high expressing cell lines (FIG. 6). IL-6, IL-4, IL-10 and MCP-1 upregulated with the higher expressing cell line.

### Example 5-7B1 T-Cell Proliferation and T Cell Phenotypes

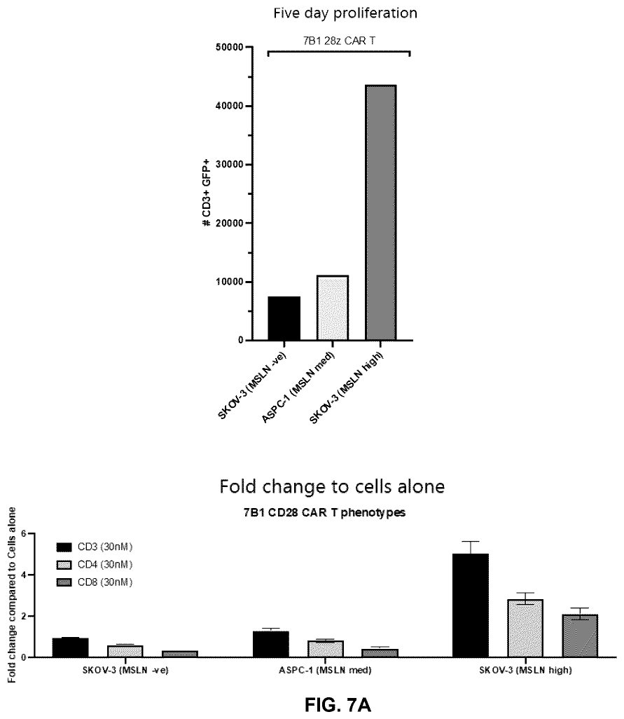

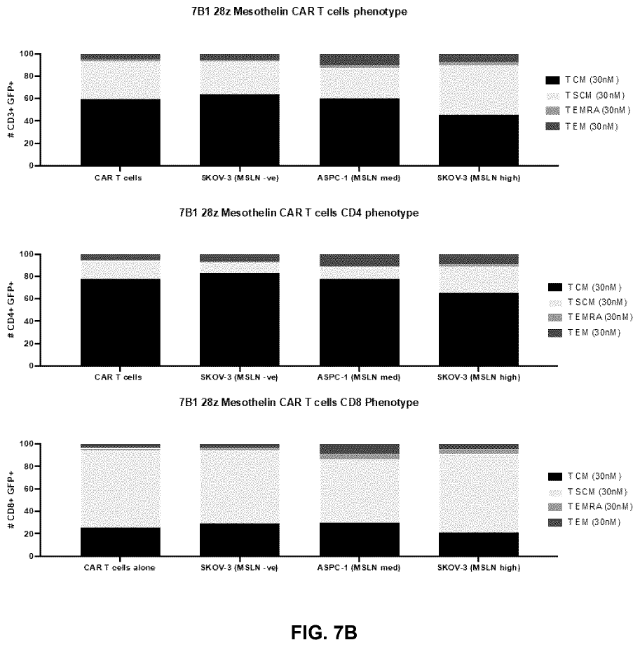

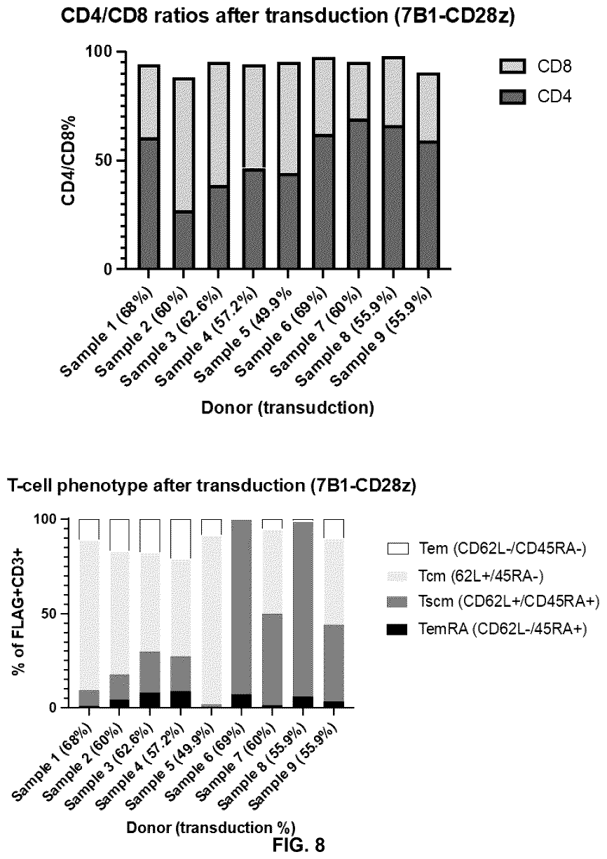

Cell lines with varying mesothelin expression (ASPC1 pancreatic and SKOV3-mesothelin transfected) and mesothelin negative cells (SKOV-3) were incubated with 7B1-28z CAR T-cells. Proliferation of CAR T-cell correlates with mesothelin expression level at 5 days (FIG. 7A). T cell, as well as CD4 and CD8 subtypes phenotypes were evaluated. High levels of mesothelin correlated to higher TSCM phenotypes, with this change mainly with the CD4+ T cells (FIG. 7B).

### Example 6—Patient and Healthy Donor 7B1 CAR Transduction

Healthy buffy pack PBMC were activated in Transact, TexMACs media and IL-7/15. On D2 healthy donor PBMC were transduced with 7B1-28z lentivirus at MOI=5. Cells were expanded for 4 days with TexMACS media and IL-7/15 and then analyzed for CAR expression on D6-9. The transduction efficiency varied from 39% to 82%. Patient CD4/CD8 ratios were consistent from batch to batch with a small amount of variability (FIG. 8A). The phenotype of healthy patient CAR T were similar, with predominantly Tcm and Tscm phenotype (FIG. 8A).

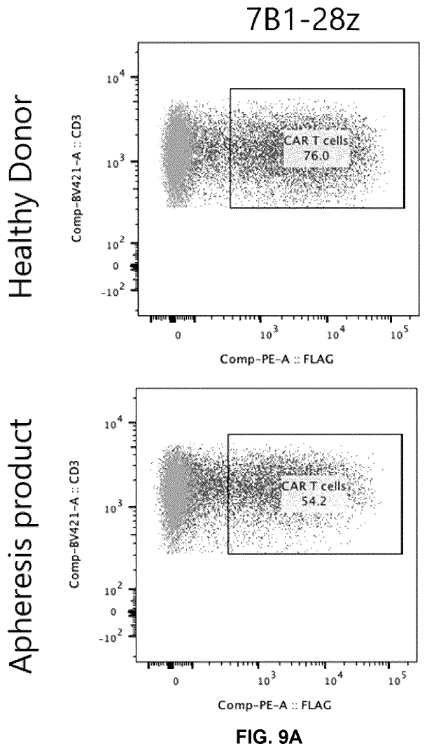

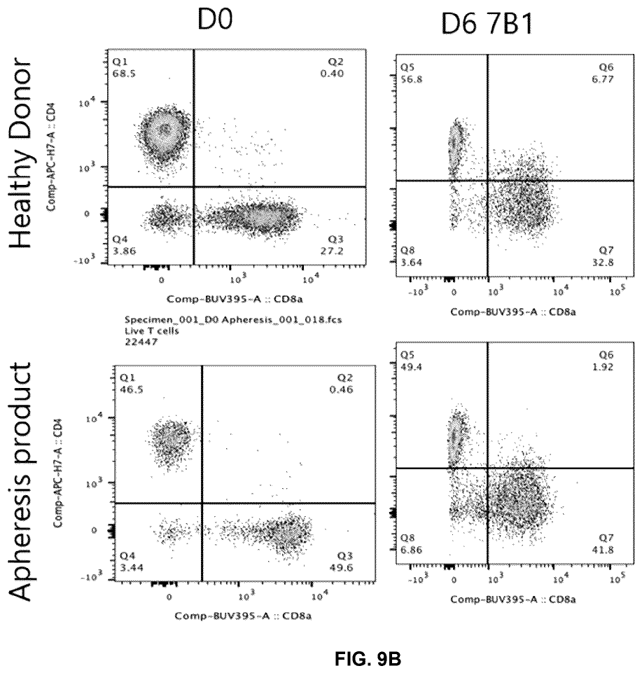

Patent apheresis and healthy buffy pack PBMC were activated in Transact and IL-7/15. On D2 patient apheresis and healthy donor PBMC were transduced with 7B1-28z lentivirus at M01=2. Cells were expanded for 4 days with TexMACS media and IL-7/15 and then analyzed for CAR expression on D6. The transduction efficiency is slightly lower for patient pheresis as compared with healthy donor PBMC as expected, 7B1-28z: 54% v 76% (FIG. 9A). Patient CD4/CD8a ratio decreased over the 6 days, while healthy donor CD4/CD8a stayed the same (FIG. 9B).

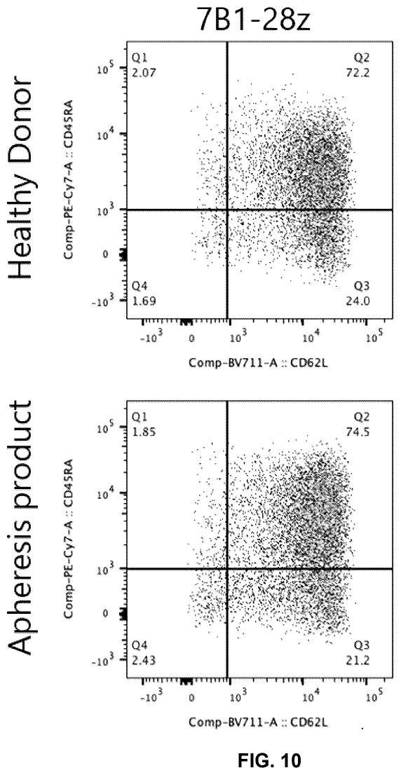

The phenotype of patient and healthy donor CAR T were very similar at day 6 timepoint (FIG. 10). The yield and fold expansion of patient CAR T was reduced compared to the healthy donor (Table 4).

TABLE 4
7B1 CAR T cell yield and expansion in patient donor cells

| | | | | Yield (CAR |
|---|---|---|---|---|
| | | CAR T | Fold | T/initial |
| Sample | Cell count | cell yield | expansion | PBMC) |
| Patent apheresis | 37 × 10⁶ | 20 × 10⁶ | 5.6× | 3.07 |
| Healthy Donor | 13 × 10⁶ | 10.3 × 10⁶ | 26× | 20.6 |

### Example 7—Patient and Healthy Donor 7B1 CAR T Cell Function

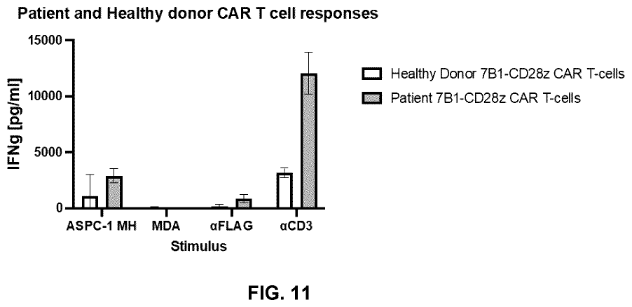

100 k CAR T were cultured in a 1:1 culture with ASPC-1 (mesothelin positive) or MDA-MB-468 (mesothelin negative) cells, or plate bound FLAG/CD3 for 24 hours. Supernatants were then analysed for IFNg secretion using Perkin Elmer AlphaLISA kit. Patient and healthy donor PBMC samples responded equivalently in the IFNg assay (FIG. 11).

### Example 8—Cytotoxicity of 7B1 CAR T Cells

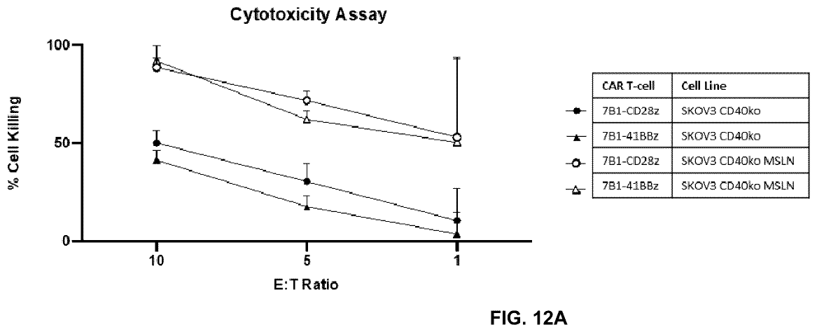

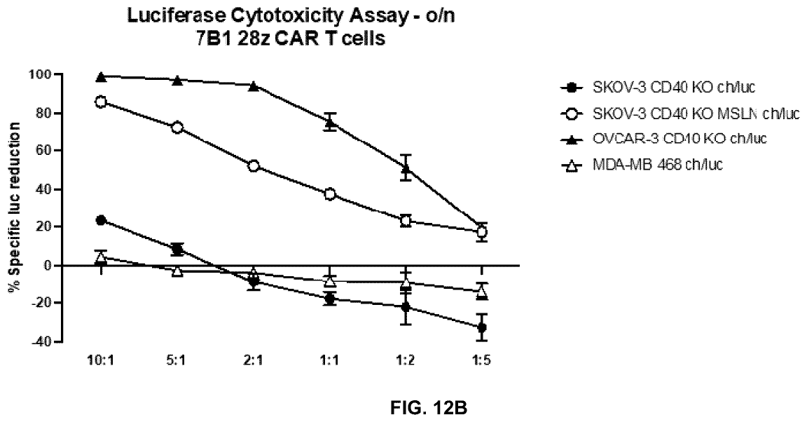

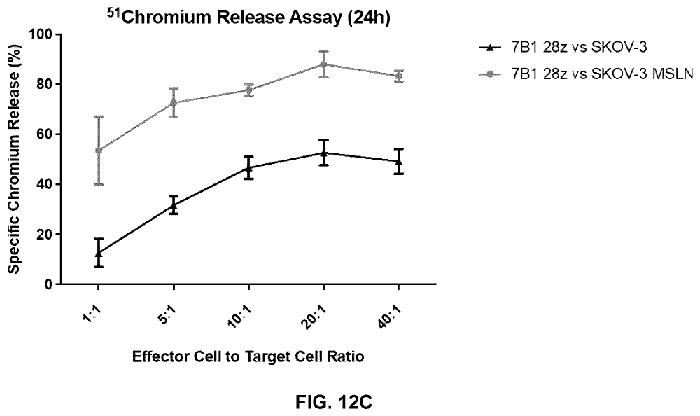

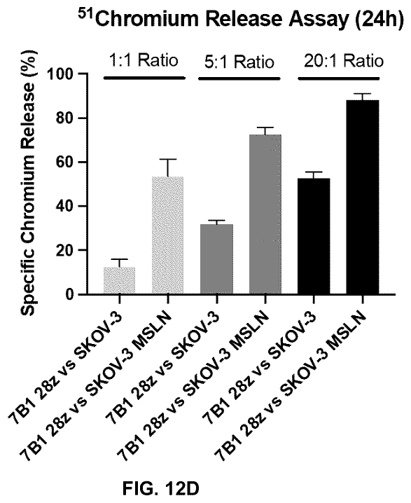

Cytolytic capacity of 7B1-28z and 7B1-41BBz was confirmed using a luciferase assay (24 h) with the SKOV-3 MESO transfected cell line in a dose response manner. CD40 was depleted in the parental and MSLN-expressing cell line to reduce the background cell killing that was not mesothelin-dependent. (FIG. 12A).

7B1-28z cytotoxicity was confirmed using a luciferase assay (24 h) with the SKOV-3 MESO-CD40 knock out transfected cell line in a dose response manner as well as with the OVCAR-3 (ovarian cancer cell line +ve for mesothelin) Cell killing was limited with the Mesothelin negative cell lines SKOV3-CD40 knock out and MDA-MB-468 (FIG. 12B).

7B1-28z cytotoxicity was confirmed using a 51-chromium release assay (24 h) with the SKOV-3 MESO transfected cell line (positive for mesothelin) compared to the parental cell line (negative for mesothelin). Cytotoxicity in a dose response manner in the mesothelin positive cell was confirmed cell killing was reduced with the Mesothelin negative cell line (FIG. 12C-D).

### Example 9-7B1-28ζ CAR T Cell Immunogenicity

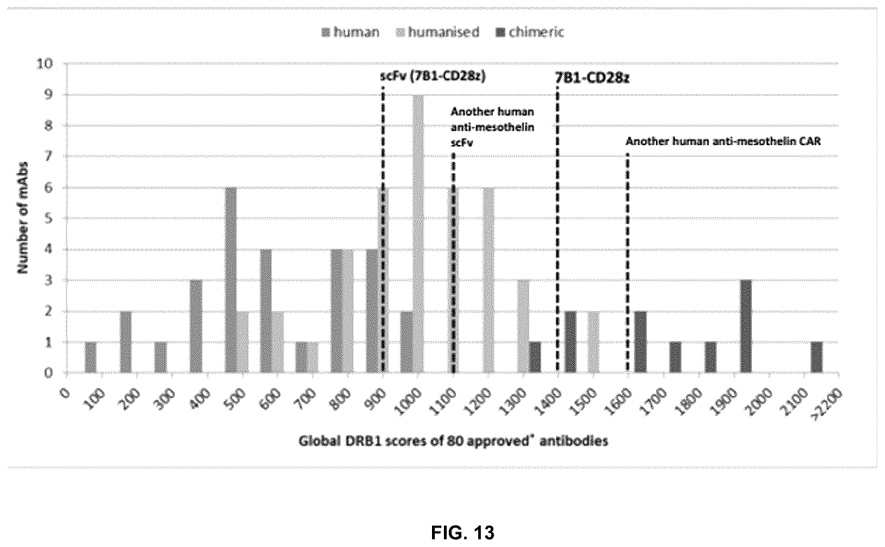

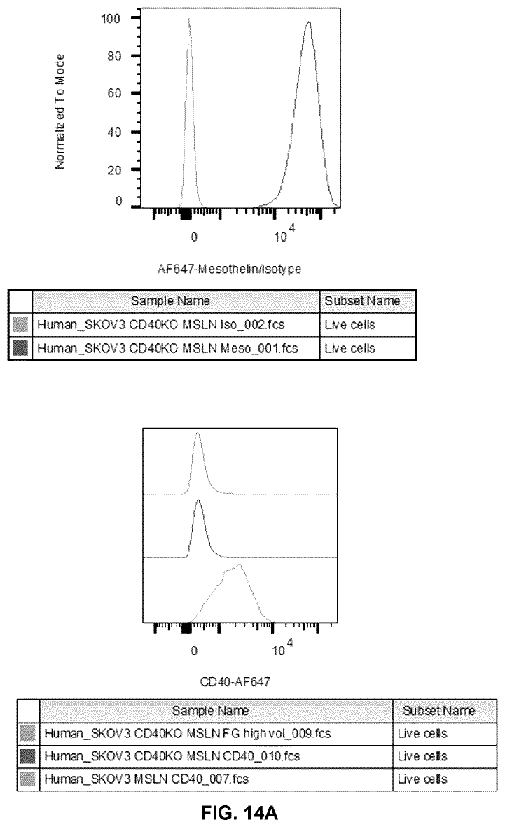

The sequence of 7B1-2K was analysed for the presence of putative HLA class I restricted epitopes, also known as T_{c} epitopes and HLA class II restricted epitopes, also known as Tₕ epitopes using Lonza's Epibase platform. Profiling was performed for 18 A and 17 B HLA allotypes, 43 DRB1, 8 DRB3/4/5, 22 DQ and 12 DP allotypes and other potential epitopes ranked by their immunogenic potential and population impact. 7B1 was ranked less immunogenic than another anti-mesothelin scFv and CAR when HLA class II epitopes considered (DRB1 HLA class II epitopes are shown in FIG. 13).

7B1-2ζ was ranked less immunogenic than another human anti-mesothelin CAR for HLA class II epitopes. Further the FLAG tag and junctions around the FLAG tag region did not contribute to immunogenicity risk.

### Example 10-7B1 CAR T Cell Activity In Vivo

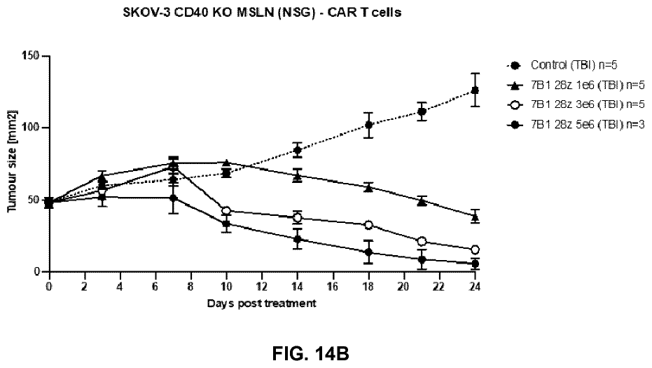

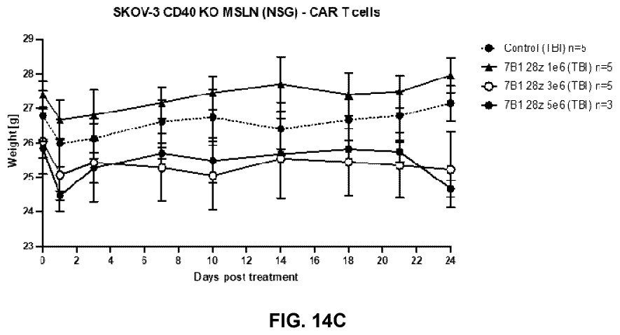

Evaluation of the 7B1-CD28z CAR in an NSG model with SKOV-3 CD40 knock out and mesothelin transfected tumour cells. Cells have confirmed mesothelin expression (FIG. 14A; top) and CD40 knocked out (FIG. 14A; bottom).

Tumours established (50 mm 3) in NSG mice using SKOV3-Mesothelin transfected cells with CD40 knocked out. A single dose of 7B1-CD28z was evaluated at three different concentrations (5×10⁶, 3×10⁶, or 1×10⁶ 7B1-CD28z CAR T cells). A dose response was seen with the 7B1-CD28z CAR (FIG. 14B). There was no significant weight loss in any treatment group over the course of the experiment (FIG. 14C).

1. A chimeric antigen receptor (CAR) comprising:
(i) an antigen recognition domain that recognises mesothelin;
(ii) a transmembrane domain; and
(iii) an intracellular signalling domain,
wherein the antigen recognition domain comprises:
(a) a light chain variable domain (VL) comprising three complementarity determining regions (CDRs) of the amino acid sequence shown in SEQ ID NO: 2; and
(b) a heavy chain variable domain (VH) comprising three complementarity determining regions (CDRs) of the amino acid sequence shown in SEQ ID NO: 1.

2. A CAR of claim 1, wherein
wherein the antigen recognition domain comprises:
(a) a light chain variable domain (VL) comprising a complementarity determining region (CDR) 1 comprising or consisting of an amino acid sequence of SEQ ID NOs: 55, 56, 57 or 58; a CDR2 comprising or consisting of an amino acid sequence of SEQ ID NO: 62, 63 or 64; a CDR3 comprising or consisting of an amino acid sequence of SEQ ID NO: 68 or 69; and
(b) a heavy chain variable domain (VH) comprising a complementarity determining region (CDR) 1 comprising or consisting of an amino acid sequence of SEQ ID NO: 25, 26, 27, 28 or 29; a CDR2 comprising or consisting of an amino acid sequence of SEQ ID NO: 34, 35, 36, 37 or 38; a CDR3 comprising or consisting of an amino acid sequence of SEQ ID NO: 45, 46, 47 or 48.

3. A CAR of claim 1, wherein the antigen recognition domain comprises:
(a) a light chain variable domain (VL) comprising a complementarity determining region (CDR) 1 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NOs: 55; a CDR2 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 62; a CDR3 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 68; and
(b) a heavy chain variable domain (VH) comprising a complementarity determining region (CDR) 1 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 27; a CDR2 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 36; a CDR3 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 45, or
(c) a light chain variable domain (VL) comprising a complementarity determining region (CDR) 1 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NOs: 55; a CDR2 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 62; a CDR3 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 68; and
(d) a heavy chain variable domain (VH) comprising a complementarity determining region (CDR) 1 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 25; a CDR2 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 34; a CDR3 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 45, or
(e) a light chain variable domain (VL) comprising a complementarity determining region (CDR) 1 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NOs: 55; a CDR2 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 62; a CDR3 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 68; and
(f) a heavy chain variable domain (VH) comprising a complementarity determining region (CDR) 1 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 26; a CDR2 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 35; a CDR3 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 45, or
(g) a light chain variable domain (VL) comprising a complementarity determining region (CDR) 1 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NOs: 56; a CDR2 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 63; a CDR3 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 69; and
(h) a heavy chain variable domain (VH) comprising a complementarity determining region (CDR) 1 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 28; a CDR2 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 37; a CDR3 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 46, or
(i) a light chain variable domain (VL) comprising a complementarity determining region (CDR) 1 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NOs: 57; a CDR2 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 64; a CDR3 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 68; and
(j) a heavy chain variable domain (VH) comprising a complementarity determining region (CDR) 1 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 29; a CDR2 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 38; a CDR3 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 47, or
(k) a light chain variable domain (VL) comprising a complementarity determining region (CDR) 1 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NOs: 58; a CDR2 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 62; a CDR3 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 68; and
(l) a heavy chain variable domain (VH) comprising a complementarity determining region (CDR) 1 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 27; a CDR2 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 36; a CDR3 comprising or consisting of an amino acid sequence of at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of SEQ ID NO: 48.

4. A CAR of claim 1, wherein the antigen recognition domain comprises:
(a) a light chain variable domain (VL) comprising a complementarity determining region (CDR) 1 comprising or consisting of an amino acid sequence of SEQ ID NOs: 55; a CDR2 comprising or consisting of an amino acid sequence of SEQ ID NO: 62; a CDR3 comprising or consisting of an amino acid sequence of SEQ ID NO: 68; and
(b) a heavy chain variable domain (VH) comprising a complementarity determining region (CDR) 1 comprising or consisting of an amino acid sequence of SEQ ID NO: 27; a CDR2 comprising or consisting of an amino acid sequence of SEQ ID NO: 36; a CDR3 comprising or consisting of an amino acid sequence of SEQ ID NO: 45, or
(c) a light chain variable domain (VL) comprising a complementarity determining region (CDR) 1 comprising or consisting of an amino acid sequence of SEQ ID NOs: 55; a CDR2 comprising or consisting of an amino acid sequence of SEQ ID NO: 62; a CDR3 comprising or consisting of an amino acid sequence of SEQ ID NO: 68; and
(d) a heavy chain variable domain (VH) comprising a complementarity determining region (CDR) 1 comprising or consisting of an amino acid sequence of SEQ ID NO: 25; a CDR2 comprising or consisting of an amino acid sequence of SEQ ID NO: 34; a CDR3 comprising or consisting of an amino acid sequence of SEQ ID NO: 45, or
(e) a light chain variable domain (VL) comprising a complementarity determining region (CDR) 1 comprising or consisting of an amino acid sequence of SEQ ID NOs: 55; a CDR2 comprising or consisting of an amino acid sequence of SEQ ID NO: 62; a CDR3 comprising or consisting of an amino acid sequence of SEQ ID NO: 68; and
(f) a heavy chain variable domain (VH) comprising a complementarity determining region (CDR) 1 comprising or consisting of an amino acid sequence of SEQ ID NO: 26; a CDR2 comprising or consisting of an amino acid sequence of SEQ ID NO: 35; a CDR3 comprising or consisting of an amino acid sequence of SEQ ID NO: 45, or
(g) a light chain variable domain (VL) comprising a complementarity determining region (CDR) 1 comprising or consisting of an amino acid sequence of SEQ ID NOs: 56; a CDR2 comprising or consisting of an amino acid sequence of SEQ ID NO: 63; a CDR3 comprising or consisting of an amino acid sequence of SEQ ID NO: 69; and
(h) a heavy chain variable domain (VH) comprising a complementarity determining region (CDR) 1 comprising or consisting of an amino acid sequence of SEQ ID NO: 28; a CDR2 comprising or consisting of an amino acid sequence of SEQ ID NO: 37; a CDR3 comprising or consisting of an amino acid sequence of SEQ ID NO: 46, or
(i) a light chain variable domain (VL) comprising a complementarity determining region (CDR) 1 comprising or consisting of an amino acid sequence of SEQ ID NOs: 57; a CDR2 comprising or consisting of an amino acid sequence of SEQ ID NO: 64; a CDR3 comprising or consisting of an amino acid sequence of SEQ ID NO: 68; and
(j) a heavy chain variable domain (VH) comprising a complementarity determining region (CDR) 1 comprising or consisting of an amino acid sequence of SEQ ID NO: 29; a CDR2 comprising or consisting of an amino acid sequence of SEQ ID NO: 38; a CDR3 comprising or consisting of an amino acid sequence of SEQ ID NO: 47, or
(k) a light chain variable domain (VL) comprising a complementarity determining region (CDR) 1 comprising or consisting of an amino acid sequence of SEQ ID NOs: 58; a CDR2 comprising or consisting of an amino acid sequence of SEQ ID NO: 62; a CDR3 comprising or consisting of an amino acid sequence of SEQ ID NO: 68; and
(l) a heavy chain variable domain (VH) comprising a complementarity determining region (CDR) 1 comprising or consisting of an amino acid sequence of SEQ ID NO: 27; a CDR2 comprising or consisting of an amino acid sequence of SEQ ID NO: 36; a CDR3 comprising or consisting of an amino acid sequence of SEQ ID NO: 48.

5-6. (canceled)

7. A CAR of claim 1, wherein the CAR further comprises an epitope or affinity tag.

8. A CAR of claim 1, wherein the CAR further comprises an epitope or affinity tag, wherein the epitope or affinity tag is C-terminal to the antigen binding domain or is located between the antigen binding domain and a transmembrane domain.

9. (canceled)

10. A CAR of claim 1, wherein the CAR further comprises an epitope or affinity tag, wherein the epitope or affinity tag is a peptide tag or a protein tag, optionally selected from any one of FLAG-tag, HA-tag, His, tag, Myc-tag, S-tag, SBP-tag, Strep-tag or eXact-tag, a GST-tag, MBP-tag or GFP-tag.

11-12. (canceled)

13. A CAR of claim 1, wherein the CAR further comprises an epitope comprising or consisting, wherein the epitope tag comprises or consists of an amino acid sequence of SEQ ID NO: 11.

14. (canceled)

15. A CAR of claim 1, wherein the CAR further comprises a hinge, wherein preferable the hinge is derived from CD8a.

16-18. (canceled)

19. A CAR of claim 1, wherein the transmembrane domain comprises a portion derived from CD8a.

20. (canceled)

21. A CAR of claim 1, wherein the signalling domain comprises a portion derived from an activation receptor such as a member of the CD3 co-receptor complex, including CD3-ζ (CD3-zeta).

22-24. (canceled)

25. A CAR of claim 1, wherein the signalling domain comprises a portion derived from a co-stimulatory receptor such as CD28 or 4-1BB (CD137).

26-28. (canceled)

29. A CAR of claim 1, wherein the CAR comprises (from N to C terminus) and antigen binding domain as described herein, a linker, an epitope or affinity tag (preferably a FLAG tag), CD8a hinge, a CD8a transmembrane domain, a CD28 signalling domain and a CD3ζ (zeta) signalling domain.

30. A CAR of claim 1, wherein the CAR comprises (from N to C terminus) and antigen binding domain as described herein, a linker, an epitope or affinity tag (preferably a FLAG tag), CD8a hinge, a CD8a transmembrane domain, a 4-1BB signalling domain and a CD3ζ (zeta) signalling domain.

31. A CAR of claim 1 wherein, the CAR comprises or consists of an amino acid sequence of SEQ ID NO: 7.

32. A CAR of claim 1, wherein the CAR comprises or consists of an amino acid sequence of SEQ ID NO: 8.

33. A nucleic acid molecule comprising a nucleotide sequence encoding a chimeric antigen receptor of claim 1.

34-42. (canceled)

43. A genetically modified cell comprising a nucleic acid molecule encoding a CAR of claim 1.

44. A genetically modified cell comprising a CAR of claim 1, or a nucleic acid molecule encoding a CAR of claim 1, wherein the cell is an immune cell.

45. (canceled)

46. A genetically modified cell comprising a CAR of claim 1 or a nucleic acid encoding a CAR of claim 1, wherein cell is a leukocyte, preferable selected from a lymphocyte, a T cell, a natural killer (NK) cell, a natural killer T cell or a tumour infiltrating lymphocyte (TIL).

47. A genetically modified cell comprising a CAR of claim 1, or a nucleic acid encoding a CAR of claim 1, wherein the cell is a CD4+ T cell or a CD8+ T cell.

48. A method of generating a genetically modified cell, said method comprising transducing the cell, preferably an immune cell, with a nucleic acid construct of claim 33, so that the transduced cell expresses the CAR, thereby generating a genetically modified cell.

49. (canceled)

50. A method of killing a cell expressing mesothelin, the method comprising exposing the cell expressing mesothelin to a genetically modified cell of claim 43, thereby killing a cell expressing mesothelin.

51. A method of claim 50, wherein the cell expressing mesothelin is a cancer cell.

52-53. (canceled)

54. A pharmaceutical composition including a genetically modified cell of claim 43 and a pharmaceutically acceptable carrier.

55-57. (canceled)
